(19) United States
(12) Patent Application Publication
(10) Pub. No.: US 2007/0258558 A1
(43) Pub. Date: Nov. 8, 2007

(54) X-RAY CT APPARATUS AND X-RAY CT SCANNING METHOD

(76) Inventors: Akihiko Nishide, Tokyo (JP); Shinichi Iisaku, Tokyo (JP); Junko Sekiguchi, Tokyo (JP)

Correspondence Address: Patrick W. Rasche;Armstrong Teasdale LLP, Suite 2600, One Metropolitan Square, St. Louis, MO, 63102, US

(21) Appl. No.: 11/738,238

(22) Filed: Apr. 20, 2007

(30) Foreign Application Priority Data
Apr. 23, 2006 (JP) 2006-118604

(51) Int. Cl. A61B 6/00; G01N 23/00; G21K 1/12; H05G 1/60

(52) U.S. Cl. 378/8

(57) ABSTRACT

The present invention provides an X-ray CT apparatus which realizes an improvement in timing control on transition to an actual scan for contrast agent synchronous imaging and its reliability, and an improvement in reduction in X-ray exposure. The X-ray CT apparatus includes an X-ray source, an X-ray detector disposed so as to be opposite to the X-ray source with a subject implanted with a contrast agent being interposed therebetween, a contrast agent synchronous imaging device which performs acquisition of projection data upon the start of the actual scan for the contrast agent synchronous photography while relative operations between the subject and the X-ray source, and the X-ray detector are being accelerated in a predetermined direction, and image reconstructing device which reconstructs a tomographic image, based on the projection data.

### CROSS REFERENCE TO RELATED APPLICATIONS

This application claims the benefit of Japanese Patent Application No. 2006-118604 filed Apr. 23, 2006.

### BACKGROUND OF THE INVENTION

The present invention relates to an X-ray CT apparatus which realizes speeding-up of timing control on contrast agent synchronous imaging in a medical X-ray CT (Computed Tomography) apparatus or the like.

Upon contrast agent synchronous imaging or photography of an X-ray CT apparatus, the mean or average CT value in each region of interest is measured at predetermined time intervals at a monitor scan for the contrast agent synchronous imaging, and the monitor scan is terminated when the average CT value exceeds a certain predetermined threshold value. Then, a cradle is moved to the position of an actual scan to carry out the actual scan. Since the actual scan uses a helical scan, the cradle has been moved to the actual scan position in consideration of to a run-up distance necessary for the cradle acceleration. Since the timing provided to start the actual scan also start after the acceleration of the cradle, it took time to start the actual scan. It is important to prevent timing provided for a contrast agent from being missed and perform the actual scan in a shorter period of time for the purpose of reducing the amount of the contrast agent upon the contrast agent synchronous photography in particular. Therefore, a problem arises in that the timing provided to start the actual scan become slower (Japanese Unexamined Patent Publication No. 2006-051234 for example).

Because of a multi-row X-ray detector or a two-dimensional X-ray area detector typified by a flat panel, the cone angle of an X-ray beam of X-ray CT apparatus has been increased. Therefore, there is a trend to more grow a problem about X-ray needless exposure. There is a tendency that as the cone angle of the X-ray beam becomes larger, a run-up distance for a helical scan at the time that a monitor scan for contrast agent synchronous imaging is shifted to an actual scan becomes also longer. Further, the timing might be missed and reliability has been demanded upon the measurement of the average CT value in the region of interest at the monitor scan for the contrast agent synchronous photography. In this case, however, there might be a case in which the timing be missed in the case of the average CT value in the whole region of interest.

### SUMMARY OF THE INVENTION

In a first aspect, an X-ray CT apparatus of the present invention includes an X-ray source, an X-ray detector disposed so as to be opposite to the X-ray source with a subject implanted with a contrast agent being interposed therebetween, a contrast agent synchronous imaging device which performs acquisition of projection data upon the start of the actual scan for the contrast agent synchronous photography while relative operations between the subject and the X-ray source, and the X-ray detector are being accelerated in a predetermined direction, and image reconstructing device which reconstructs a tomographic image, based on the projection data.

In the X-ray CT apparatus according to the first aspect, an accelerating operation is performed in a predetermined direction, i.e., a z-axis direction corresponding to the direction of relative operations between a subject and an X-ray tube, and an X-ray detector upon the relative operations between the subject and X-ray tube, and the X-ray detector at the start of the actual scan for the contrast agent synchronous photography. When a scan gantry including the X-ray tube and the X-ray detector is kept static, a cradle with the subject placed thereon is accelerated in the z-axis direction to start a variable pitch helical scan for an actual scan. Thus, the time up to the start of actual scan photography is shortened as compared with the case in which a run-up section is provided so far to accelerate the cradle in the z-axis direction, and thereafter a helical scan is started after a constant velocity is reached. Consequently, the shortening of a time interval up to the start of the actual scan for the contrast agent synchronous photography can be realized.

In a second aspect, contrast agent synchronous imaging device accelerates the relative operations between the subject and the X-ray source, and the X-ray detector from a static state after the start of acquisition of the projection data.

In an X-ray CT apparatus according to the second aspect, X-ray data acquisition is performed from a static state in a predetermined direction, corresponding to relative operations between a subject and an X-ray tube, and an X-ray detector upon the relative operations between the subject and X-ray tube, and the X-ray detector at the start of the actual scan for the contrast agent synchronous photography. Then, the X-ray CT apparatus enters an accelerating operation. That is, when a scan gantry including the X-ray tube and the X-ray detector is kept static, X-ray data acquisition is started in a state in which a cradle with the subject placed thereon is placed in a static state. Then, a variable pitch helical scan for an actual scan is started and an accelerating operation is reached after a predetermined time. Thus, the time up to the start of actual scan photography is shortened as compared with the case in which a run-up section is provided so far to accelerate the cradle in a predetermined direction, and thereafter the helical scan is started after a constant velocity is reached. As shown in FIG. 38B, a tomogram image reconstructable range can be extended outside by half an X-ray beam width at maximum than a moving range for each of the X-ray tube and the X-ray detector. That is, it is thus possible to realize a tomogram image reconstruction range making the best use of an X-ray radiation range and realize a reduction in exposure.

In a third aspect, according to an X-ray CT apparatus of the present invention, the image reconstructing device image-reconstructs, as a position for image-reconstructing a tomographic image, up to a range extended outside by half an X-ray beam width of an X-ray data acquisition system than a z-direction moving range at a center position of the X-ray data acquisition system.

In an X-ray CT apparatus according to the third aspect, when X-ray data acquisition is performed from a static state and an accelerating operation is reached, it is possible to rotate an X-ray tube and an X-ray detector by at least fan angles+180° within an xy plane under the static state to thereby perform a conventional scan (axial scan) and to perform image reconstruction using X-ray projection data of a portion of an X-ray beam, corresponding to half an X-ray beam width at maximum, lying outside a moving range for each of the X-ray tube and the X-ray detector. Incidentally, the X-ray tube and the X-ray detector enter X-ray data acquisition at a variable pitch helical scan subsequent to the conventional scan (axial scan).

In the static state, even when the X-ray tube and the X-ray detector enter the X-ray data acquisition for the variable pitch helical scan after the rotation of fan angels+180° or less within the xy plane, image reconstruction corresponding to a range equivalent to half or less of the X-ray beam width lying outside the moving range for each of the X-ray tube and the X-ray detector can be performed. That is, owing to these, a tomogram image reconstruction range that makes the best use of an X-ray radiation range can be realized, and a reduction in X-ray exposure can be realized.

In a fourth aspect, contrast agent synchronous imaging device acquires projection data while relative operations between a subject and an X-ray source, and an X-ray detector are being decelerated in a predetermined direction, upon the completion of an actual scan.

In an X-ray CT apparatus according to the fourth aspect, X-ray data acquisition is performed while carrying out a decelerating operation in a predetermined direction corresponding to the direction of relative operations between a subject and an X-ray tube, and an X-ray detector upon the relative operations between the subject and X-ray tube, and the X-ray detector at the end of the actual scan for the contrast agent synchronous photography. The X-ray data acquisition is also completed after the completion of the z-axis relative operations for the actual scan. That is, when a scan gantry including the X-ray tube and X-ray detector is kept static in the predetermined direction, X-ray data acquisition is performed even when a decelerating operation is reached after a cradle with the subject placed thereon has been operated at a constant velocity in the predetermined direction. When the cradle stops after the end of a predetermined direction operation, the X-ray data acquisition is also terminated. Thus, the time up to the start of actual scan photography is shortened as compared with the case in which a run-up section is provided so far to accelerate the cradle in the z-axis direction, and thereafter a helical scan is started after a constant velocity is reached. That is, consequently, the shortening of a time interval up to the start of the actual scan for the contrast agent synchronous photography can be realized.

In a fifth aspect, contrast agent synchronous imaging device acquires projection data while relative operations between a subject and an X-ray source, and an X-ray detector are being decelerated in a predetermined direction, upon the completion of an actual scan, acquires the projection data for a predetermined time after the relative operations have been stopped, and thereafter completes the acquisition of the projection data.

In an X-ray CT apparatus according to the fifth aspect, a decelerating operation in a predetermined direction corresponding to the direction of relative operations between a subject and an X-ray tube, and an X-ray detector is performed upon the relative operations between the subject and X-ray tube and the X-ray detector at the end of the actual scan for the contrast agent synchronous imaging. Thereafter, X-ray data acquisition is performed until a certain predetermined time elapses even after a static state is reached, after which the X-ray data acquisition is terminated. That is, when a scan gantry including the X-ray tube and the X-ray detector is kept static, X-ray data acquisition is performed even when a decelerating operation is reached after a cradle with the subject placed thereon has been operated at a constant velocity in the predetermined direction. Further, the X-ray data acquisition is performed until a predetermined time interval elapses after the cradle ends its predetermined direction operation and stops, after which the X-ray data acquisition is ended. Thus, the time up to the start of actual scan photography is shortened as compared with the case in which a run-up section is provided so far to accelerate the cradle in the predetermined direction, and thereafter a helical scan is started after a constant velocity is reached. That is, consequently, the shortening of a time interval up to the start of the actual scan for the contrast agent synchronous photography can be realized.

In a sixth aspect, contrast agent synchronous imaging device performs deceleration in a predetermined direction at an actual scan and performs acceleration again, and continuously performs the acquisition of projection data.

In an X-ray CT apparatus according to the sixth aspect, when a one-direction relative operation for the actual scan for the contrast agent synchronous photography is terminated, X-ray data acquisition is performed while a decelerating operation in a predetermined direction corresponding to the direction of relative operations between a subject and an X-ray tube, and an X-ray detector is being performed. Further, the X-ray data acquisition is thereafter carried out while an accelerating operation is being performed. That is, when a scan gantry including the X-ray tube and the X-ray detector is kept static in the predetermined direction, X-ray data acquisition is carried out while the operation of decelerating a cradle with the subject placed thereon in the predetermined direction is being performed. Further, the X-ray data acquisition is thereafter carried out while the cradle is performing an accelerating operation at a predetermined direction operation. Thus, the time up to the start of actual scan photography is shortened as compared with the case in which a run-up section is provided so far to accelerate the cradle in the predetermined direction, and thereafter a helical scan is started after a constant velocity is reached. That is, consequently, the shortening of a time interval up to the start of the actual scan for the contrast agent synchronous photography can be realized.

In a seventh aspect, contrast agent synchronous imaging device decelerates relative operations for an actual scan in a predetermined direction and further accelerates the same again after their stop, and continuously performs the acquisition of projection data.

In an X-ray CT apparatus according to the seventh aspect, X-ray data acquisition is performed while a decelerating operation in a predetermined direction corresponding to the direction of relative operations between a subject and an X-ray tube, and an X-ray detector is being performed. Even though a static state is reached, the X-ray data acquisition is carried out for a certain predetermined time. Thereafter, the X-ray data acquisition is performed while an accelerating operation is carried out. That is, when a scan gantry including the X-ray tube and the X-ray detector is kept static in the predetermined direction, X-ray data acquisition is carried out while a cradle with the subject placed thereon is performing a decelerating operation in the predetermined direction. Even though the cradle is brought to the static state, the X-ray data acquisition is performed for a certain predetermined time. Thereafter, the X-ray data acquisition is performed even while the cradle is performing the accelerating operation. Thus, the time up to the start of actual scan photography is shortened as compared with the case in which a run-up section is provided so far to accelerate the cradle in the predetermined direction, and thereafter a helical scan is started after a constant velocity is reached. That is, consequently, the shortening of a time interval up to the start of the actual scan for the contrast agent synchronous photography can be realized.

In an eighth aspect, image reconstructing device in an X-ray XCT apparatus of the present invention performs a three-dimensional image reconstructing process.

In the X-ray CT apparatus according to the eighth aspect, a tomogram image reconstruction range can be extended outside by half an X-ray beam width at maximum than a moving range for each of an X-ray tube and an X-ray detector as shown in FIG. 38B by using three-dimensional image reconstruction. Simultaneously with it, image reconstruction can be performed without so deteriorating the quality of a tomographic image lying outside the moving range of each of the X-ray tube and the X-ray detector.

In a ninth aspect, image reconstructing device performs image reconstruction using a coordinate position in a predetermined direction, of projection data, which is obtained by measuring a position in a predetermined direction, of the projection data by coordinate measuring device or predicting a coordinate position in a predetermined direction by relative operations between a subject and an X-ray tube, and an X-ray detector controlled in advance.

In an X-ray CT apparatus according to the ninth aspect, X-ray data acquisition is performed even where an accelerating operation is carried out in the predetermined direction corresponding to the direction of operation of X-ray data acquisition relative to the subject, or even from a static state. The coordinate measuring device measures a coordinate position in the predetermined direction, of X-ray projection data. Alternatively, the coordinate measuring device predicts coordinate positions of relative operations between the subject and X-ray tube and the X-ray detector controlled in advance. Owing to it, three-dimensional image reconstruction can be done using higher-precision predetermined direction coordinates, and a tomographic image that is better in image quality and less reduced in artifact is obtained.

In a tenth aspect, one collimator capable of moving in a traveling direction extending along the predetermined direction from the center connecting an X-ray source and an X-ray detector between the X-ray source and the X-ray detector, and the other collimator capable of moving in a traveling direction opposite to the predetermined direction from the center are provided. The other collimator is moved in a non-traveling direction from the center side upon the start of an actual scan, and the one collimator is moved from the traveling direction to the center upon the end of the actual scan.

In an X-ray CT apparatus according to the tenth aspect, the aperture of one collimator as viewed in the traveling direction of the X-ray tube and the X-ray detector is made broader than the aperture of the rear other collimator on the opposite side thereof upon relative operations between the subject and X-ray tube and the X-ray detector when X-ray data acquisition is started, thereby to carry out X-ray data acquisition. Alternatively, upon the completion of the X-ray data acquisition, the aperture of one collimator in the traveling direction is set narrower than that of the rear other collimator to perform X-ray data acquisition.

That is, consider where an accelerating operation/decelerating operation is performed in a predetermined direction from a state in which the cradle with the subject placed thereon is at rest, to thereby perform a variable pitch helical scan. As shown in FIG. 39, the aperture of the other collimator on the side opposite to the traveling direction of the cradle is kept closed upon the start of X-ray data acquisition. The X-ray data acquisition is performed while the aperture of the other collimator is being opened depending upon the degree of traveling of the cradle. As shown in FIG. 39 as well, the aperture of one collimator on the traveling direction side of the cradle is kept closed depending upon the degree of traveling of the cradle upon the end of the X-ray data acquisition. Upon the completion of the X-ray data acquisition, the X-ray data acquisition is carried out in such a manner that the aperture of one collimator is closed by half.

Thus, a moving range, an X-ray radiation range and a tomogram image reconstructable range of the X-ray tube and X-ray detector at a variable pitch helical scan or a helical shuttle scan become equal to one another. Hence, an X-ray needless exposure region becomes nonexistent. That is, it is thus possible to realize a tomogram image reconstruction range that makes the best use of the X-ray radiation range and realize a reduction in exposure.

In an eleventh aspect, contrast agent synchronous imaging device tilts an X-ray source and an X-ray detector with respect to a subject at the start of an actual scan in such a manner that a boundary in a direction opposite to the traveling direction of the predetermined direction, of an X-ray beam emitted from the X-ray source becomes orthogonal to the predetermined direction, and tilts the X-ray source and the X-ray detector with respect to the subject at the end of the actual scan in such a manner that a boundary in the traveling direction, of an X-ray beam emitted from the X-ray source becomes orthogonal to the predetermined direction.

In an X-ray CT apparatus according to the eleventh aspect, the X-ray tube and the X-ray detector are tilted at the start of X-ray data acquisition. The boundary of an X-ray beam on the side opposite to the direction of traveling of the X-ray tube and X-ray detector at the relative operations between the subject and the X-ray tube and the X-ray detector is set approximately orthogonal to the predetermined direction. Further, the boundary of an X-ray beam lying in the direction of traveling of the X-ray tube and X-ray detector is set approximately orthogonal to the predetermined direction at the end of the X-ray data acquisition. Thus, the shape of the X-ray beam for the X-ray tube and X-ray detector becomes rectangular within a yz plane as shown in FIG. 44B. In this case, the shape of the X-ray beam for the X-ray tube and X-ray detector results in the minimum X-ray radiation shape. The most tomogram image reconstruction therein is performed and a reduction in X-ray exposure can be realized efficiently.

In a twelfth aspect, according to an X-ray CT apparatus of the present invention, contrast agent synchronous imaging device performs a monitor scan for observing the injection of a contrast agent to a subject before an actual scan, and an X-ray beam width for the monitor scan is narrower than an X-ray beam width for the actual scan.

Upon the monitor scan for the contrast agent synchronous photography in this case, a CT value of each pixel in a region of interest for starting up the actual scan, or an average or mean CT value of the pixels therein is simply measured. Hence, a broad X-ray beam wide in range as viewed in the predetermined direction is unnecessary. Therefore, a reduction in X-ray exposure at the monitor scan can be realized by using the minimum X-ray beam width at which SN can be ensured on an image-quality basis in width narrower than at the actual scan.

In a thirteenth aspect, according to an X-ray CT apparatus of the present invention, contrast agent synchronous imaging device performs a monitor scan for observing the injection of a contrast agent to a subject before an actual scan, and performs the actual scan when the maximum value of values of pixels or the mean of plural pixel values thereof selected in decreasing order from the maximum value, the pixels being pixels that belong to a region of interest set by the monitor scan, exceeds a threshold value.

In the X-ray CT apparatus according to the thirteenth aspect, the region of interest set at each monitor scan for the contrast agent synchronous imaging is set to a vascular portion in which the contrast agent flows and each CT value is likely to rise, and is used to start up the actual scan for the contrast agent synchronous imaging. The actual scan has heretofore been started up where the average CT value in the region of interest becomes larger than a certain predetermined threshold value. There is a case in which in the region of interest at the monitor scan at this time, a trigger is not applied well depending upon the setting of the region of interest when the average CT value is used. In order to avoid it and apply the trigger reliably, the maximum value of each pixel in the region of interest, or the mean value of plural pixels selected in decreasing order from the maximum value is used, and the trigger may be applied when it exceeds a given threshold value. When the CT value of each pixel for the vascular portion contained in the region of interest rises due to the arrival of the contrast agent, then the degree of certainty of application of the trigger is raised if the maximum value of the pixels in the region of interest is checked.

Looking at the average value of plural pixels selected in decreasing order from the maximum value makes it possible to suppress variations in the CT value of each pixel and apply a trigger stably. That is, the trigger for the actual scan can be applied stably by these methods.

In a fourteenth aspect, according to an X-ray CT apparatus of the present invention, contrast agent synchronous imaging device performs a monitor scan for observing the injection of a contrast agent to a subject before an actual scan, and performs the actual scan when the mean of pixel values in a two-dimensional continuous region, of pixels belonging to a region of interest set by the monitor scan, or the area of the two-dimensional continuous region exceeds a threshold value.

In the X-ray CT apparatus according to the fourteenth aspect, the region of interest set at each monitor scan for the contrast agent synchronous imaging is set to a vascular portion in which the contrast agent flows and each CT value is likely to rise, and the actual scan for the contrast agent synchronous imaging is started up. There is a case in which in the region of interest at the monitor scan at this time, a trigger is not applied well depending upon the setting of the region of interest when the average CT value is used. In order to avoid it and start up stably, the average CT value of pixels contained in a two-dimensional continuous region containing pixels having the maximum CT value in a region of interest corresponding to the size of each blood vessel, and the area of the two-dimensional continuous region are checked. A trigger for the actual scan can be applied depending upon whether they exceed a given threshold value.

Thus, looking at the average CT value of pixels contained in the continuous region corresponding to the blood vessels makes it possible to prevent a malfunction that the value of each pixel other than the blood vessel happens to exceed a threshold value and the trigger for the actual scan is applied. That is, the actual scan can be started up stably by these methods.

In a fifteenth aspect, according to an X-ray CT apparatus of the present invention, contrast agent synchronous imaging device performs a monitor scan for observing the injection of a contrast agent to a subject before an actual scan, and performs the actual scan when the mean of pixel values in a three-dimensional continuous region, of pixels belonging to a region of interest set by the monitor scan, or the volume of the three-dimensional continuous region exceeds a threshold value.

In order to start up the actual scan reliably, when a plurality of sheets of tomographic images are photographed in a predetermined direction at the monitor scan, the average CT value of pixels contained in a three-dimensional continuous region corresponding to the size of each blood vessel as the three-dimensional continuous region, and the volume of the three-dimensional continuous region are checked. A trigger for the actual scan can be applied depending on whether they exceed a given threshold value. Thus, setting regions of interest over a plurality of sheets of tomographic images without setting one sheet of tomographic image and looking at CT of pixels contained in the three-dimensional continuous region containing pixels having the maximum CT value in a region of interest corresponding to the size of each blood vessel make it possible to prevent a malfunction that each pixel value other than the blood vessel happens to exceed a threshold value, thereby applying a trigger for the actual scan. That is, the trigger for the actual scan can be applied stably.

In a sixteenth aspect, according to an X-ray CT apparatus of the present invention, contrast agent synchronous imaging device performs a monitor scan for observing the injection of a contrast agent to a subject before an actual scan, and the two or more regions of interest set by the monitor scan are included.

When a region of interest set at a monitor scan for contrast agent synchronous photography is set plural, for example, one region of interest is set to vascular portions in which a contrast agent flows and each CT value is likely to rise to begin with. Another region of interest is set to an organ to be subjected to contrast and then photographed, i.e., such a portion as to be first stained with the contrast agent. An actual scan for contrast agent synchronous imaging is started up after CT values in two regions of interest exceed their corresponding threshold values sufficiently. Thus, when the trigger for the actual scan is applied in the plural regions of interest after it is confirmed that the contrast agent has been introduced sufficiently, the actual scan startup can reliably be applied as compared with the case in which only one region of interest is set. Confirming the attainment of the contrast agent to the plural regions of interest in this way makes it possible to prevent a malfunction that the value of each pixel other than the blood vessel happens to exceed a threshold value and the actual scan startup is applied.

In a seventeenth aspect, according to an X-ray CT apparatus of the present invention, contrast agent synchronous imaging device performs a monitor scan for observing the injection of a contrast agent to a subject before an actual scan, and the two or more regions of interest set by the monitor scan are included in a predetermined direction.

When a plurality of sheets of tomographic images, e.g., four sheets of tomographic images are photographed in the predetermined direction at the monitor scan where a region of interest set at a monitor scan for contrast agent synchronous photography is set plural in the predetermined direction, for example, one region of interest is set to vascular portions in which a contrast agent for the first sheet of tomographic image flows and each CT value rises to begin with. Another one region of interest is set to vascular portions in which a contrast agent for a fourth sheet of tomographic image flows and a CT value is likely to rise next, in succession to the rise in CT value of the blood vessel in the region of interest for the first sheet of tomographic image.

After it is confirmed that the CT values in the two regions of interest have been raised one by one, the actual scan for the contrast agent synchronous imaging is started up. That is, the maximum CT value in the region of interest for the first sheet of tomographic image, or the average value of plural pixels selected in decreasing order from the maximum value is used to thereby confirm that it exceeds a given threshold value. Thereafter, the maximum value in the region of interest for the fourth sheet of tomographic image, or the average value of plural pixels selected in decreasing order from the maximum value is used to thereby confirm that it exceeds a given threshold value, whereby the actual scan is started up. Thus, it is confirmed that the contrast agent has sequentially been introduced into the plural regions of interest different in predetermined direction position of the tomographic image, and the actual scan is started up. Therefore, the actual scan can reliably be started up as compared with the case in which one region of interest is provided. Confirming the attainment of the contrast agent in proper order and in suitable delay time at the plural regions of interest different in position in the predetermined direction in this way makes it possible to prevent a malfunction that the value of each pixel other than the blood vessel happens to exceed a threshold value and the startup of the actual scan is applied.

In an eighteenth aspect, according to an X-ray CT apparatus of the present invention, upon a monitor scan, the acquisition of projection data is performed while relative operations between a subject and an X-ray source, and an X-ray detector are at least in acceleration or deceleration.

Where a plurality of sheets of tomographic images, e.g., four sheets of tomographic images are photographed in the predetermined direction at the monitor scan when a region of interest set at a monitor scan for contrast agent synchronous photography is set plural in the predetermined direction, for example, one region of interest is set to vascular portions in which a contrast agent for the first sheet of tomographic image flows and each CT value rises to begin with. Another one region of interest is set to vascular portions in which a contrast agent for a fourth sheet of tomographic image flows and a CT value is likely to rise next, in succession to the rise in CT value of the blood vessel in the region of interest for the first sheet of tomographic image. When plural coordinate positions in the predetermined direction are separated from one another, there is a need to skip therebetween, preferably, in a short period of time and perform tomographic image photography by a reciprocating operation. If it is not so, then each intermittent scan for such a monitor scan as shown in FIG. 25 cannot be realized. In this case, short-time intermittent scans can be carried out by performing tomographic image photography as in the helical shuttle scan even in acceleration or deceleration.

In a nineteenth aspect, an X-ray CT apparatus of the present invention performs, upon a monitor scan, acquires projection data while relative operations between a subject and an X-ray source, and an X-ray detector are in acceleration from a static state or in a static state subsequent to deceleration.

Where a plurality of sheets of tomographic images, e.g., four sheets of tomographic images are photographed in the predetermined direction at the monitor scan when a region of interest set at a monitor scan for contrast agent synchronous photography is set plural in the predetermined direction, for example, one region of interest is set to vascular portions in which a contrast agent for the first sheet of tomographic image flows and each CT value rises to begin with. Another one region of interest is set to vascular portions in which a contrast agent for a fourth sheet of tomographic image flows and a CT value is likely to rise next, in succession to the rise in CT value of the blood vessel in the region of interest for the first sheet of tomographic image. When plural coordinate positions in the predetermined direction are separated from one another, there is a need to skip therebetween, preferably, in a short period of time and perform tomographic image photography by a reciprocating operation. If it is not so, then each intermittent scan for such a monitor scan as shown in FIG. 25 cannot be realized. When some time allowance exists in the interval between the intermittent scans in this case, a standstill or static state corresponding to a predetermined period may be inserted into a period of transition from deceleration to acceleration. Thus, advantages are brought about in that no trouble occurs on a mechanical control basis, body movements of the subject are less reduced, and the image quality for the monitor scan, particularly, artifacts are reduced. It is thus possible to realize a stable monitor scan good in image quality.

The X-ray CT apparatus of the present invention has advantageous effects that can bring about in that an improvement in timing control on the transition to an actual scan for constant agent synchronous imaging at a helical scan or a variable pitch helical scan or a helical shuttle scan, an improvement in reliability of a monitor scan, and a reduction in exposure can be realized.

### BRIEF DESCRIPTION OF THE DRAWINGS

FIG. 1 is a block diagram showing an X-ray CT apparatus according to one embodiment of the present invention.

FIG. 2 is a diagram illustrating an imaging condition input screen of the X-ray CT apparatus.

FIG. 3A is an XY plane of a geometrical arrangement or layout of an X-ray tube 21 and a multi-row X-ray detector 24, and FIG. 3B is a YZ plane thereof.

FIG. 4 is a flowchart showing a flow for subject imaging.

FIG. 5 is a flowchart illustrating an image reconstructing schematic operation of the X-ray CT apparatus according to one embodiment of the present invention.

FIG. 6 is a flowchart showing the details of a pre-process.

FIG. 7 is a flowchart depicting the details of a three-dimensional image reconstructing process.

FIGS. 8a, 8b, 8c, and 8d are conceptual diagrams showing a state in which lines on a reconstruction area are projected in an X-ray penetration direction.

FIG. 9 is a conceptual diagram illustrating lines projected onto an X-ray detector plane.

FIG. 10 is a flowchart for describing a flow of a process for contrast agent synchronous imaging.

FIG. 11A is a diagram showing a baseline tomographic image, and FIG. 11B is a diagram showing a display example of monitor scans MS for contrast agent synchronous imaging.

FIG. 12 is a flowchart of a monitor scan MS1.

FIG. 13 is a diagram showing an intermittent scan of the monitor scan MS.

FIG. 14 is a diagram illustrating the scanning of a region of interest POI.

FIG. 15 is a flowchart showing a maximum pixel value retrieval and a comparison between a maximum pixel value and a threshold value.

FIG. 16A is a flowchart illustrating image processing of an example 1 of a monitor scan MS and FIG. 16B is a diagram showing a histogram measurement result and a maximum pixel value.

FIG. 17 is a flowchart of a monitor scan MS2.

FIG. 18 is a flowchart showing N maximum pixel value retrievals, and comparisons between N maximum pixel values and a threshold value.

FIG. 19A is a flowchart showing image processing of an example 2 of a monitor scan MS, and FIG. 19B is a diagram showing a histogram measurement result and N maximum pixels.

FIG. 20 is a flowchart of a monitor scan MS3.

FIG. 21 is a diagram showing the scanning of a two-dimensional continuous region lying within a two-dimensional region of interest.

FIG. 22 is a flowchart showing a maximum pixel value retrieval lying in a two-dimensional continuous region, and a comparison between a maximum pixel value and a threshold value.

FIG. 23A is a flowchart showing image processing of an example 3 of a monitor scan MS, and FIG. 23B is a diagram showing a maximum value pixel and two-dimensional continuous regions.

FIG. 24 is a flowchart of a monitor scan MS4.

FIG. 25 is a diagram showing the scanning of a three-dimensional continuous region lying in a three-dimensional region of interest.

FIG. 26 is a flowchart showing a maximum pixel value retrieval lying in the three-dimensional continuous region, and a comparison between a maximum pixel value and a threshold value.

FIG. 27 is a flowchart showing image processing of an example 4 of a monitor scan MS.

FIG. 28 is a flowchart of a monitor scan MS5.

FIG. 29 is a flowchart of a monitor scan MS6.

FIG. 30 is a flowchart of a monitor scan MS7.

FIG. 31 is a diagram showing intermittent scans for two monitor scans MS in a z-axis direction.

FIG. 32 is a diagram showing an operation example 1 from a monitor scan MS to an actual scan between a cradle 12, an X-ray tube 21 and a multi-row X-ray detector 24.

FIG. 33 is a diagram illustrating an operation example 2 from a monitor scan MS to an actual scan between the cradle 12, X-ray tube 21 and multi-row X-ray detector 24.

FIG. 34 is a diagram showing an operation example 3 from a monitor scan MS to an actual scan between the cradle 12, X-ray tube 21 and multi-row X-ray detector 24.

FIG. 35 is a diagram depicting an operation example 4 from a monitor scan MS to an actual scan between the cradle 12, X-ray tube 21 and multi-row X-ray detector 24.

FIG. 36 is a diagram showing an operation example 5 from a monitor scan MS to an actual scan between the cradle 12, X-ray tube 21 and multi-row X-ray detector 24.

FIG. 37 is a diagram illustrating an operation example 6 from a monitor scan MS to an actual scan between the cradle 12, X-ray tube 21 and multi-row X-ray detector 24.

FIG. 38A is a diagram showing a moving range and a tomogram image reconstructable range of an X-ray tube 21 and a multi-row X-ray detector 24 at a variable pitch helical scan where an X-ray collimator 23 is half-closed at the start and end of X-ray data acquisition, and FIG. 38B is a diagram showing a moving range and a tomogram image reconstructable range of the X-ray tube 21 and the multi-row X-ray detector 24 at the variable pitch helical scan.

FIG. 39 is a diagram illustrating X-ray collimator control for reducing X-ray exposure at the start and end of imaging.

FIG. 40 is a flowchart for describing control on the X-ray collimator 23 at X-ray data acquisition.

FIG. 41A is a diagram showing the operation of the cradle 12 subjected to velocity linear control, FIG. 41B is a diagram showing an X-ray tube current thereof, FIG. 41C is a diagram showing the operation of the cradle 12 subjected to velocity non-linear control, and FIG. 41D is a diagram showing an X-ray tube current thereof.

FIGS. 42a and 42b are diagrams illustrating an X-ray beam at each position of the X-ray collimator 23.

FIG. 43 is a diagram showing the output of a collimator position detection channel 75 at each collimator position.

FIG. 44A is a diagram showing an X-ray irradiation range at a helical scan, and FIG. 44B is a diagram showing an X-ray irradiation range at a helical scan at the time that a scan gantry 20 is tilted.

FIG. 45 is a diagram showing an operation example 7 from a monitor scan MS to an actual scan between the cradle 12, X-ray tube 21 and multi-row X-ray detector 24.

FIG. 46 is a diagram illustrating an operation example 8 from a monitor scan MS to an actual scan between the cradle 12, X-ray tube 21 and multi-row X-ray detector 24.

FIG. 47 is a diagram depicting an operation example 9 from a monitor scan MS to an actual scan between the cradle 12, X-ray tube 21 and multi-row X-ray detector 24.

FIG. 48 is a diagram showing an operation example 10 from a monitor scan MS to an actual scan between the cradle 12, X-ray tube 21 and multi-row X-ray detector 24.

### DETAILED DESCRIPTION OF THE INVENTION

The present invention will hereinafter be explained in further detail by embodiments illustrated in the figures. Incidentally, the present invention is not limited to or by the embodiments.

<Overall Configuration of X-Ray CT Apparatus>

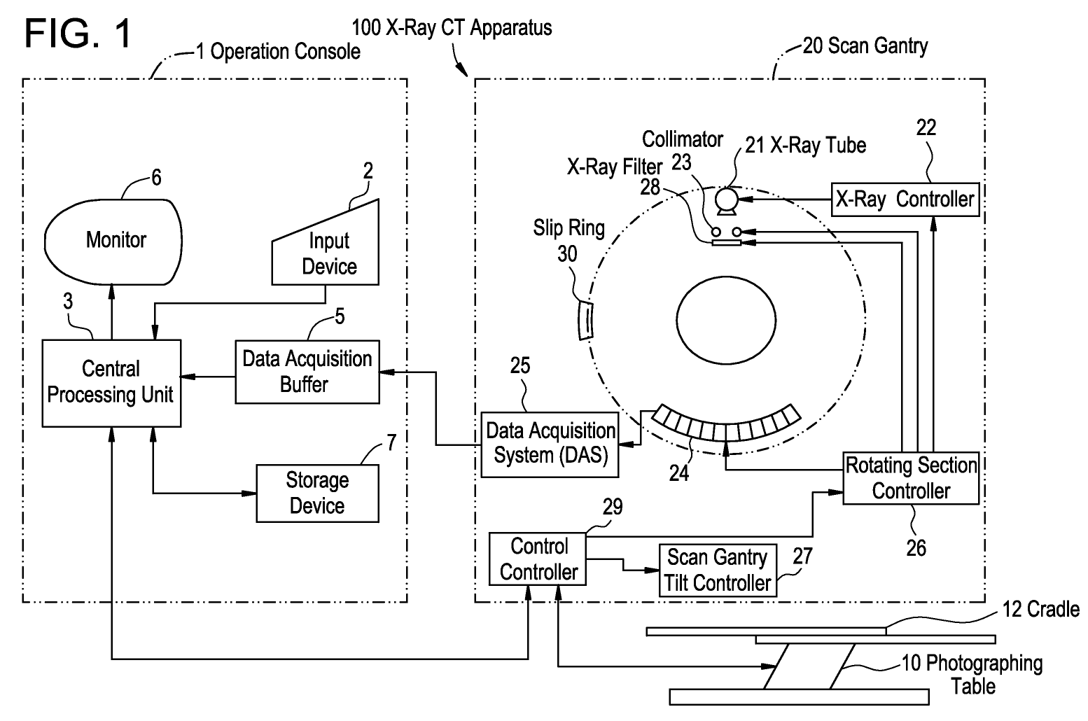

FIG. 1 is a configuration block diagram showing an X-ray CT apparatus according to one embodiment of the present invention. The X-ray CT apparatus 100 is equipped with an operation console 1, an imaging or photographing table 10 and a scan gantry 20.

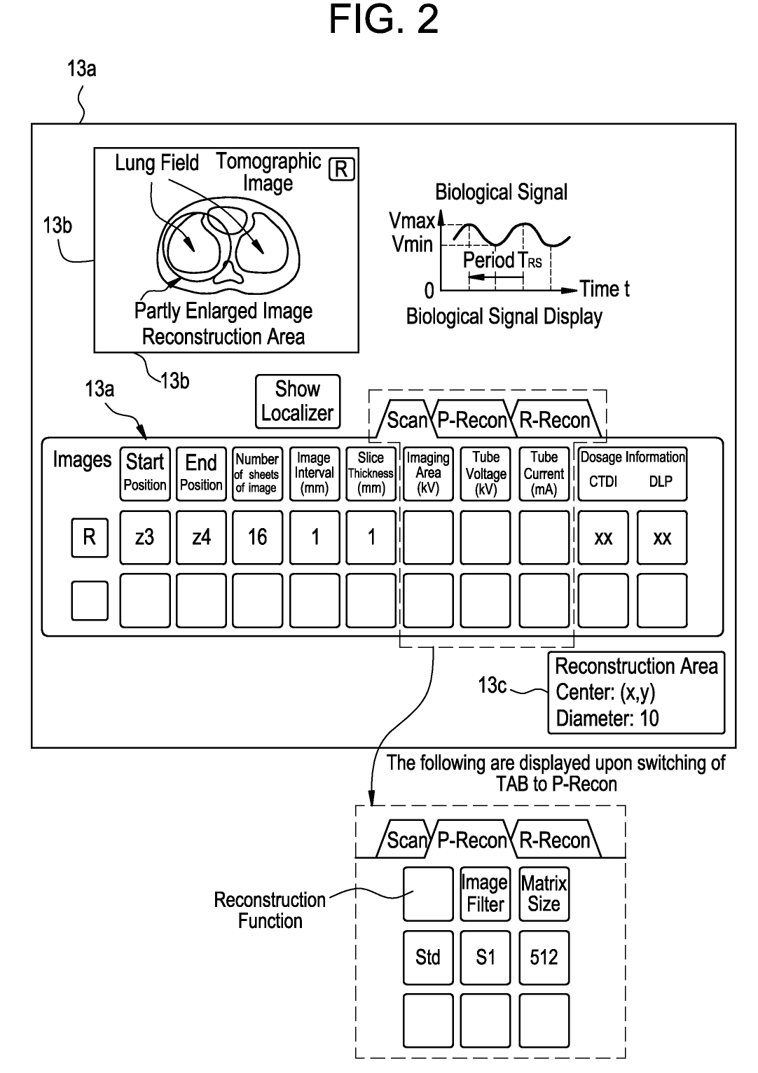

The operation console 1 includes an input device 2 which accepts an input from an operator, a central processing unit 3 which executes a pre-process, an image reconstructing process, a post-process, etc., a data acquisition buffer 5 which acquires or collects X-ray detector data acquired by the scan gantry 20, a monitor 6 which displays a tomographic image image-reconstructed from projection data obtained by pre-processing the X-ray detector data, and a storage device 7 which stores programs, projection data and X-ray tomographic images therein. Imaging or photographing conditions is inputted to the device 2 and stored in the device 7. FIG. 2 shows an example of an imaging condition input screen 13A displayed on the monitor 6. An input button 13a for performing a predetermined input is displayed on the imaging condition input screen 13A. FIG. 2 illustrates a screen on which a scan tab is being selected. When P-Recon is selected as the tab, the display is switched to an input display. A tomographic image 13b is displayed above the input button 13a and a reconstruction area 13c is displayed down below. Biological signals may be displayed as displayed on the upper right side if necessary.

The photographing table 10 of FIG. 1 includes a cradle 12 that draws and inserts a subject from and into a bore or aperture of the scan gantry 20 with the subject placed thereon. The cradle 12 can be elevated and moved on the photographing table by a motor built in the photographing table 10.

The scan gantry 20 includes an X-ray tube 21, an X-ray controller 22, a collimator 23, a beam forming X-ray filter 28, a multi-row X-ray detector 24, a data acquisition system (DAS) 25, a rotating section controller 26 which controls the X-ray tube 21 or the like rotated about a body axis of the subject, and a control controller 29 which swaps control signals or the like with the operation console 1 and the photographing table 10. The beam forming X-ray filter 28 is an X-ray configured so as to be thinnest in thickness thereof as viewed in the direction of X rays directed to the center of rotation, to increase in thickness thereof toward its peripheral portion and to be able to further absorb the X rays. This is because that sectional shape reduce exposure to radiation on the body surface of the subject which nearly circular or elliptic. The scan gantry 20 can be tiled about ±30° or so forward and rearward as viewed in a z-axis direction by a scan gantry tilt controller 27.

The X-ray tube 21 and the multi-row X-ray detector 24 are rotated about the center of rotation IC. Rotation plane of the X-ray tube 21 and the multi-row X-ray detector 24 is a xy plane, and the direction in which the cradle 12 is moved, corresponds to the z-axis direction.

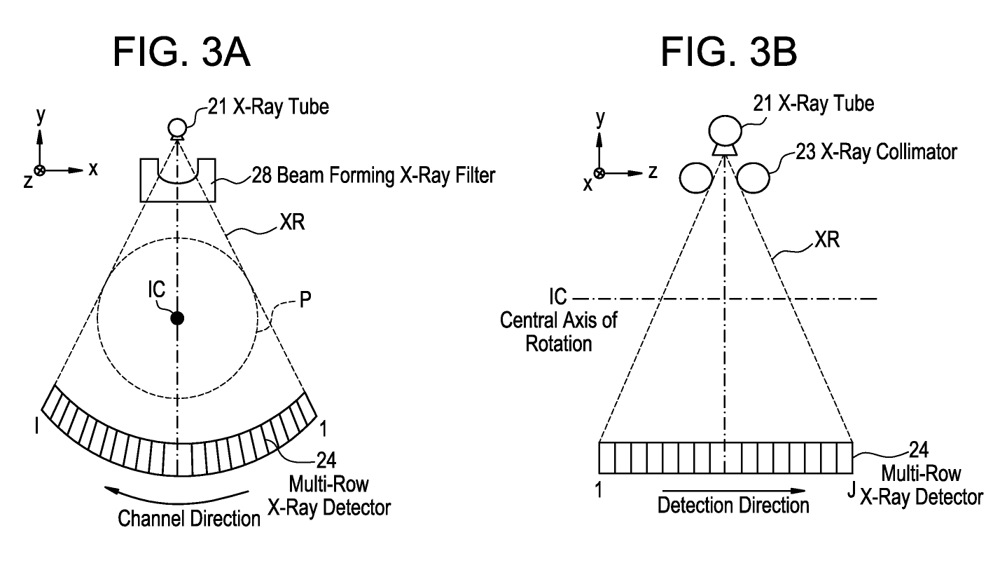

FIGS. 3A and 3B are diagrams showing geometrical arrangements or layouts of the X-ray tube 21 and the multi-row X-ray detector 24. The X-ray tube 21 generates an X-ray beam XR called a cone beam. When the direction of a central axis of the cone beam is parallel to the y direction, this is defined as a view angle 0°. The multi-row X-ray detector 24 has X-ray detector rows corresponding to J rows, for example, 256 rows as viewed in the z-axis direction. Each of the X-ray detector rows has X-ray detector channels corresponding to I channels, for example, 1024 channels as viewed in a channel direction. In FIG. 3A, an X-ray beam is set such that area P by the beam forming X-ray filter 28 and less X rays are irradiated at a peripheral portion thereof thereby. After X-ray dosage has been spatially controlled in this way, the X rays are absorbed into a subject, and the penetrated X rays are acquired or collected by the multi-row X-ray detector 24 as X-ray detector data.

In FIG. 3B, the X-ray beam XR is controlled in the direction of slice thickness of a tomographic image by the X-ray collimator 23. Acquired projection data are A/D converted by the data acquisition system 25 from the multi-row X-ray detector 24, which in turn are inputted to the data acquisition buffer 5 via a slip ring 30. The data inputted to the data acquisition buffer 5 are processed by the central processing unit 3 so that the data are image-reconstructed as a tomographic image and displayed on the monitor 6. Incidentally, although the multi-row X-ray detector 24 is applied in the present embodiment, a two-dimensional X-ray area detector of a matrix structure typified by a flat panel X-ray detector can also be applied, or a one-row type X-ray detector can be applied.

<Operation Flowchart of X-Ray CT Apparatus>

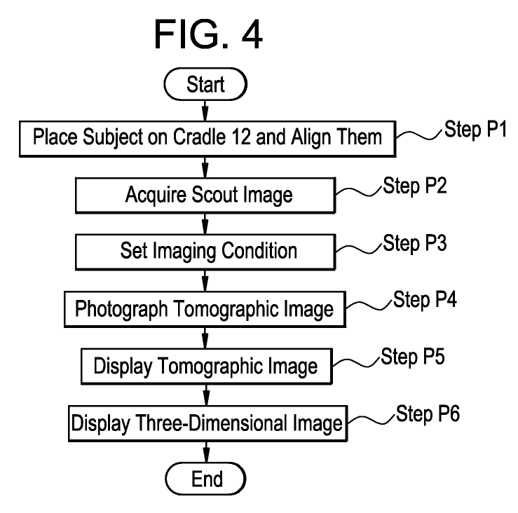

FIG. 4 is a flowchart showing the rough outline of operation of the X-ray CT apparatus according to the present embodiment.

At Step P1, a subject is placed on its corresponding cradle 12 and their alignment is performed. In the subject placed on the cradle 12, a slice light central position of the scan gantry 20 is aligned with a reference point of its each portion or region.

At Step P2, scout image (called also “scano image or X-ray penetrated image”) acquisition is performed. As the scout images, two types of scout images of an adult or children can be imaged or photographed depending upon the size of the body of the subject. Further, the scout image can be normally imaged or photographed at 0° and 90°. Only the 90° scout image might be taken depending upon the region as in the case of a head, for example. The operation of fixing the X-ray tube 21 and the multi-row X-ray detector 24 and effecting data acquisition of X-ray detector data while the cradle 12 is being linearly moved, is performed upon scout image photography.

At Step P3, an imaging condition setting is performed while the position and size of a tomographic image to be photographed on the scout image is being displayed. The present embodiment has a plurality of scan patterns such as a conventional scan (axial scan), a helical scan, a variable pitch helical scan, a helical shuttle scan, etc. The conventional scan is a scan method of rotating the X-ray tube 21 and the X-ray detector 24 each time the cradle 12 is moved by a predetermined pitch in a z-axis direction, thereby acquiring projection data. The helical scan is a scan method of moving the cradle 12 at a predetermined velocity in a state in which the X-ray tube 21 and the X-ray detector 24 are being rotated, thereby acquiring projection data. The variable pitch helical scan is a scan method of varying the speed or velocity of the cradle 12 while the X-ray tube 21 and the X-ray detector 24 are being rotated even at acceleration and deceleration, thereby acquiring projection data. The helical shuttle scan is a scan method of reciprocating the cradle 12 in the Z-axis or −Z-axis direction while the X-ray tube 21 and the X-ray detector 24 are being rotated even at acceleration and deceleration, thereby to acquire projection data. When setting up the scan, information about the whole X-ray dosage is displayed. When the number of rotations or time is inputted upon a cine scan, information about X-ray dosage corresponding to the inputted number of rotations or the inputted time at its region of interest is displayed.

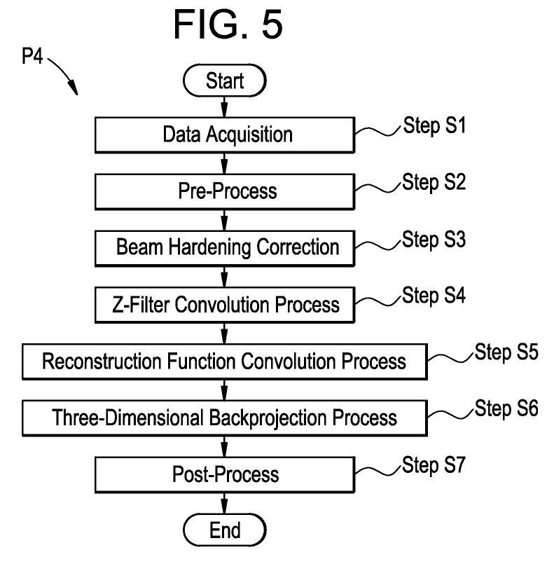

At Step P4, tomographic image photography is performed. The details of the tomographic image photography and its image reconstruction will be explained later using FIG. 5. At Step P5, the image-reconstructed tomographic image is displayed. At Step P6, a three-dimensional image display is performed using a tomographic image continuously photographed in the z-axis direction as a three-dimensional image.

(Operation Flowchart for Tomographic Image Photography and Scout Image Photography)

FIG. 5 is a flowchart showing rough outlines of operations for the tomographic image photography and scout image photography of the X-ray CT apparatus 100 of the present invention.

At Step S1, a helical scan is that a z-axis direction position Ztable(view) is added to X-ray detector data D0(view, j, i) (where j=1 to ROW, and i=1 to CH) indicated by a view angle view, a detector row number j and a channel number i, thereby carrying out data acquisition. The z-axis direction position may be added to X-ray projection data or may be used in association with the X-ray projection data as another file. Information about the z-axis direction position is used where the X-ray projection data is three-dimensionally image-reconstructed. In addition, z-axis direction position can be used for an improvement in the accuracy of an image-reconstructed tomographic image and an improvement in its quality Upon the scout image photography, the operation of fixing the X-ray tube 21 and the multi-row X-ray detector 24 and data acquisition while the cradle 12 placed on the photographing table 10 is being linearly moved, is performed.

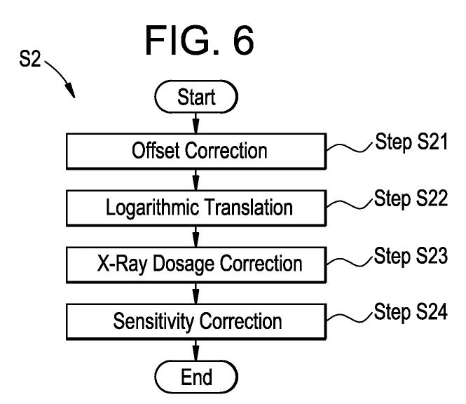

At Step S2, a pre-process is performed on the X-ray detector data D0(view, j, i) to convert it into projection data. FIG. 6 shows a specific process about the pre-process at Step S2. At Step S21, an offset correction is performed. At Step S22, logarithmic translation is performed. At Step S23, an X-ray dosage correction is performed. At Step S24, a sensitivity correction is performed. In the case of the scout image photography, the pre-processed X-ray detector data is completed as a scout image if a pixel size as viewed in the channel direction and a pixel size as viewed in the z-axis direction corresponding to the linear traveling direction of the cradle 12 are displayed in match with the display pixel size of the monitor 6.

Referring back to FIG. 5, a beam hardening correction is effected on the pre-processed projection data D1(view, j, i) at Step S3. Assuming that upon the beam hardening correction at Step S3, projection data subjected to the sensitivity correction of Step S24 of the pre-process S2 is defined as D1(view, j, i) and data subsequent to the beam hardening correction of Step S3 is defined as D11(view, j, i), the beam hardening correction is expressed in the form of, for example, a polynomial as shown below (Equation 1). Incidentally, a multiplication operation or computation is expressed in “” in the present specification.

[1]

D11(view,j,i)=D1(view,j,i)(Bo(j,i)+B₁(j,i)D1(view,j,i)+B₂(j,i)D1(view,j,i)²) (Equation 1)

Since, at this time, beam hardening corrections independent of one another every j row of detector can be performed, the differences in X-ray energy characteristics of the detectors for every row can be corrected if tube voltages of respective data acquisition systems are different on the imaging conditions.

At Step S4, a z-filter convolution process for applying filters in the z-axis direction (row direction) is effected on the projection data D11(view, j, i) subjected to the beam hardening correction. For example, a z-filter convolution process is that such row-direction filter sizes as expressed in the following equations (Equation 2 and Equation 3) are five rows, in the row direction.

(w1(i), w2(i), w3(i), w4(i), w5(i)) (Equation 2)

where (2) is given as follows.

[2]

$${\sum }_{k = 1}^{5} {w}_{k} ( i ) = 1 \qquad ( Equation 3 )$$

The corrected detector data D12(view, j, i) is expressed as follows (given by the following equation 4):

[3]

$$D 12 ( view , j , i ) = {\sum }_{k = 1}^{5} ( D 11 ( view , j + k - 3 , j ) \cdot {w}_{k} ( j ) ) \qquad ( Equation 4 )$$

Incidentally, assuming that the maximum value of the channel is CH and the maximum value of the row is ROW, the following equations (Equations 5 and 6) are given.

[4]

D11(view,−1,i)=D11(view,0,i)=D11(view,1,i) (Equation 5)

[5]

D11(view,ROW,i)=D11(view,ROW+1,i)=D11(view,ROW+2,i) (Equation 6)

When row-direction filter coefficients are changed for every channel, slice thicknesses can be controlled depending upon the distance from an image reconstruction center. In a tomographic image, its peripheral portion generally becomes thick in slice thickness than the reconstruction center thereof. The slice thicknesses can also be made constant by changing the filter coefficients at the central and peripheral portions, in order to fix it. When, for example, the row-direction filter coefficients are changed extensively in width in the neighborhood of a central channel, and the row-direction filter coefficients are changed narrowly in width in the neighborhood of a peripheral channel, each slice thickness can be made uniform even at the peripheral portion and image reconstruction central portion.

By slightly thickening the slice thickness by the row-direction filters, both artifact and noise are greatly improved. That is, the row-direction filter can control the three-dimensionally image-reconstructed tomographic image, i.e., the image quality in the xy plane. As another correction process, a tomographic image having a thin slice thickness can also be realized by subjecting the row-direction (z-axis direction) filter coefficients to deconvolution filters.

At Step S5, a reconstruction function convolution process is performed. That is, a reconstruction function convolution process is that projection data is subjected to Fast Fourier transformation (FFT) for performing transformation into a frequency domain or region and multiplied by a reconstruction function, followed by being subjected to inverse Fourier transformation. Assuming that upon the reconstruction function convolution process S5, projection data subsequent to the z filter convolution process is defined as D12, projection data subsequent to the reconstruction function convolution process is defined as D13, and the convoluting reconstruction function can be defined as Kernel(j). The reconstruction function convolution process can be expressed as follows (Equation 7). Incidentally, a convolution computation or operation is expressed in “*” in the present specification.

[6]

D13(view,j,i)=D12(view,j,i)*Kernel(j) (Equation 7)

That is, since the reconstruction function kernel (j) can perform reconstruction function convolution processes independent of one another for every j row of detector, the difference between noise characteristics set for every row and the difference between resolution characteristics can be corrected.

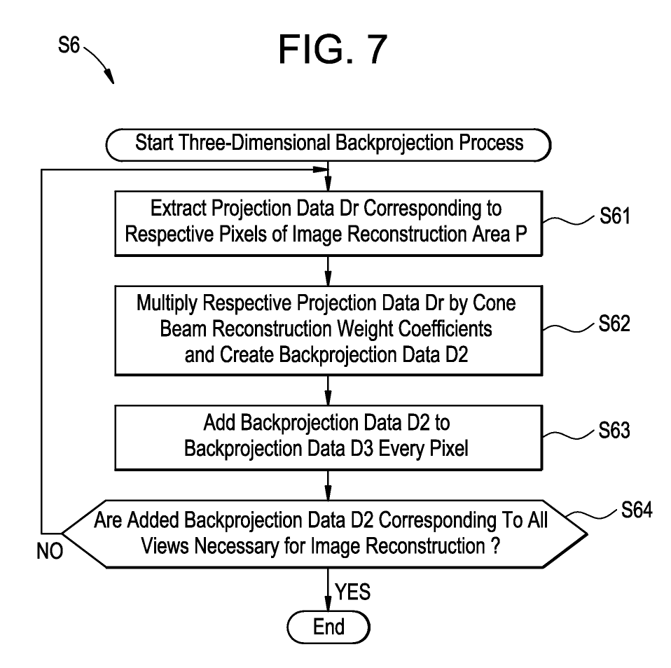

At Step S6, a three-dimensional backprojection process is effected on the projection data D13(view, j, i) the reconstruction function convolution process to determine backprojection data D3(x, y, z). A reconstructed image is three-dimensionally image-reconstructed on a plane, i.e., an xy plane orthogonal to the z axis. A reconstruction area or plane P to be shown below is assumed to be parallel to the xy plane. The three-dimensional backprojection process will be explained later referring to FIG. 7.

At Step S7, a post-process including, CT value conversion and the like is effected on the backprojection data D3(x, y, z) to obtain a tomographic image D31(x, y, z). data subsequent to the image filter convolution of D32(x, y, z), the following equation (Equation 8) is established. However, Filter(z) is the convolution two-dimensional image filter at the xy plane.

[7]

D32(x,y,z)=D31(x,y,z)*Filter(z) (Equation 8)

That is, since the image filter convolution processes independently, tomographic image at each z-coordinate position can carry out the differences between noise characteristics and between resolution characteristics for every row can be corrected.

An image space z-axis direction filter convolution process shown below may be carried out after the two-dimensional image filter convolution process. This image space z-axis direction filter convolution process may be performed before the two-dimensional image filter convolution process. Further, a three-dimensional image filter convolution process may be performed to produce such an effect as to share both of the two-dimensional image filter convolution process and the image space z-axis direction filter convolution process.

Assuming that upon the image space z-axis direction filter convolution process, a tomographic image subjected to the image space z-axis direction filter convolution process is defined as D33 (x, y, z) and a tomographic image subjected to the two-dimensional image filter convolution process is defined as D32 (x, y, z), the following equation (Equation 9) is established as follows. In the equation (9), however, v(i) indicates an image space z-axis direction filter coefficient at which a z-axis direction width is 2l+1. v(i) is expressed in the form of such a coefficient row as shown below (Equation 10).

[8]

$$D 33 ( x , y , z ) = {\sum }_{i = - 1}^{l} D 32 ( x , y , z + i ) \cdot v ( i ) \qquad ( Equation 9 )$$

[9]

v(−l), v(−l+1), . . . v(−l)v(0), v(l), . . . v(l−1), v(l) (Equation 10)

Incidentally, upon the helical scan, the image space filter coefficient v(i) may be an image space z-axis direction filter coefficient independent upon the z-axis direction position. However, when the conventional scan (axial scan) or cine scan is performed using the multi-row X-ray detector 24 or the two-dimensional X-ray area detector or the like broad in detector width in the z-axis direction in particular, the image space z-axis direction filter coefficient v(i) may preferably use an image space z-axis direction filter coefficient that depends upon the position of each X-ray detector row in the z-axis direction. This is because it is further effective since detailed adjustments dependent on the row position of each tomographic image can be made.

(Flowchart for Three-Dimensional Backprojection Process)

FIG. 7 shows the details of Step S6 shown in FIG. 6 and is a flowchart showing the three-dimensional backprojection process. In the present embodiment, an image to be image-reconstructed is three-dimensionally image-reconstructed on a plane, i.e., an xy plane orthogonal to the z axis. The following reconstruction area P is assumed to be parallel to the xy plane.

At Step S61, attention is paid to one of all views (i.e., views corresponding to 360° or views corresponding to “180°+fan angles”) necessary for image reconstruction of each tomographic image. Projection data Dr corresponding to respective pixels in the reconstruction area P are extracted.

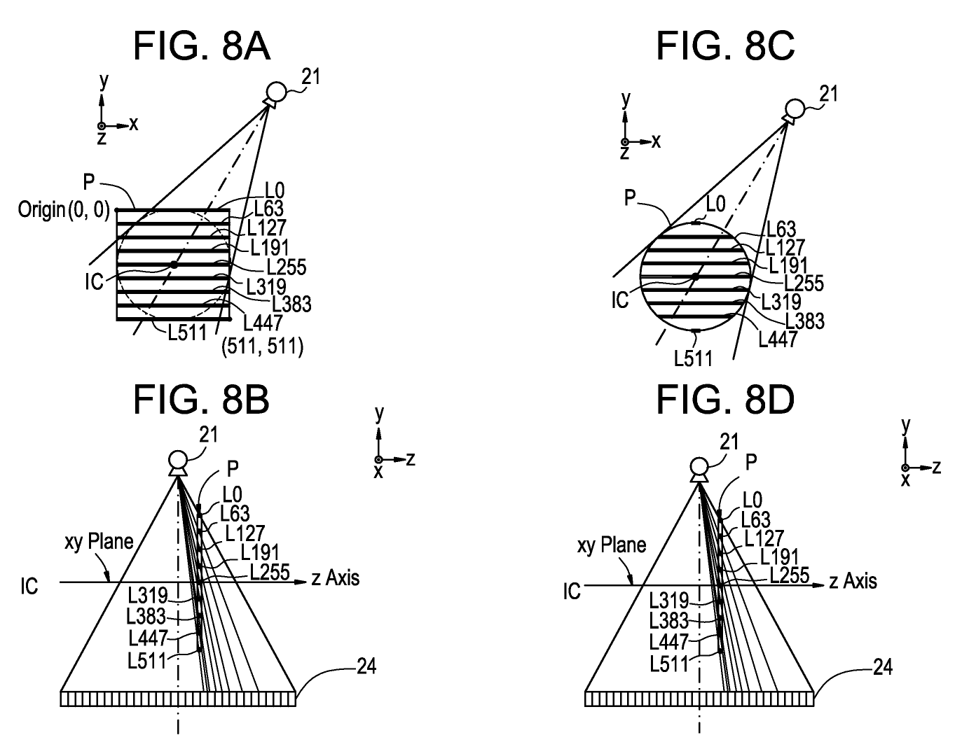

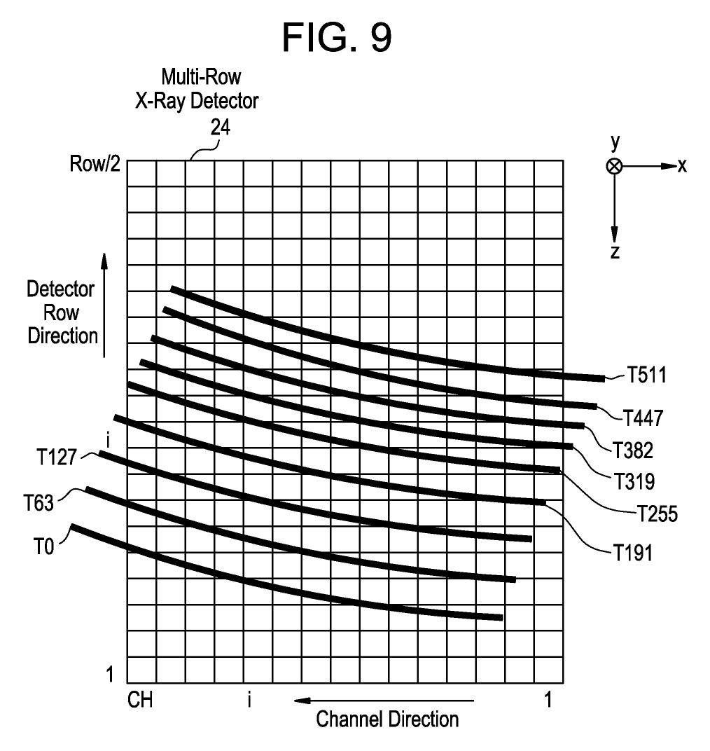

The projection data Dr will now be explained using FIGS. 8 and 9. FIGS. 8A and 8B are conceptual diagrams each showing a state in which lines on a rectangular reconstruction area are projected in an X-ray penetration direction, wherein FIG. 8A shows an xy plan and FIG. 8B shows a yz plane, respectively. FIGS. 8C and 8D are conceptual diagrams each showing a state in which lines on a circular image reconstruction area are projected in an X-ray penetration direction, wherein FIG. 8C shows an xy plane and FIG. 8D shows a yz plane, respectively. FIG. 9 is a conceptual diagram showing lines projected onto an X-ray detector plane. As shown in FIG. 8, a square area of 512×512 pixels, which is parallel to the xy plane, is assumed to be a reconstruction area P. A pixel row L0 parallel to the x axis of y=0, a pixel row L63 of y=63, a pixel row L127 of y=127, a pixel row L191 of y=191, a pixel row L255 of y=255, a pixel row L319 of y=319, a pixel row L383 of y=383, a pixel row L447 of y=447, and a pixel row L511 of y=511 are taken as rows. If projection data on lines T0 through T511 shown in FIG. 9 obtained by projecting the pixel rows L0 through L511 onto the plane of the multi-row X-ray detector 24 in an X-ray penetration direction are extracted, then they result in projection data Dr(view, x, y) of the pixel rows L0 through L511. However, x and y correspond to the respective pixels (x, y) of the tomographic image.

The X-ray penetration direction is determined depending on geometrical positions of the X-ray focal point of the X-ray tube 21, the respective pixels and the multi-row X-ray detector 24. Since, however, the z coordinates z(view) of X-ray detector data D0(view, j, i) are known with being added to the X-ray detector data as a table linear movement z-axis direction position Ztable(view), the X-ray penetration direction can be accurately determined within the X-ray focal point and the data acquisition geometrical system of the multi-row X-ray detector even in the case of the X-ray detector data D0(view, j, i) placed under acceleration and deceleration.

Incidentally, when some of lines are placed out of the multi-row X-ray detector 24 as viewed in the channel direction as in the case of, for example, the line T0 obtained by projecting the pixel row L0 on the plane of the multi-row X-ray detector 24 in the X-ray penetration direction, the corresponding projection data Dr(view, x, y) is set to “0”. When it is placed outside the multi-row X-ray detector 24 as viewed in the z-axis direction, the corresponding projection data Dr(view, x, y) is determined as extrapolation.

Referring back to FIG. 7, at Step S62, the projection data Dr(view, x, y) are multiplied by a cone beam reconstruction weight coefficient. Now, the cone beam reconstruction weight function w(i, j) is as follows. Generally, when the angle which a linear line connecting the focal point of the X-ray tube 21 and a pixel g(x, y) on the reconstruction area P (xy plane) at view=βa forms with a center axis Bc of an X-ray beam is assumed to be γ and its opposite view is assumed to be view=βb in the case of fan beam image reconstruction, the following equation (Equation 11) is established.

βb=βa+180°−2γ (Equation 11)

When the angles which the X-ray beam passing through the pixel g(x, y) on the reconstruction area P and its opposite X-ray beam form with the reconstruction plane P, are assumed to be αa and αb, backprojection pixel data D2(0, x, y) is that they are multiplied by con beam reconstruction weight coefficients ωa and ωb dependant on these and added together. In this case, the following equation (Equation 12) is given.

D2(0,x,y)=ωa·D2(0,x,y)_{—}a+ωb·D2(0,x,y)_{—}b (Equation 12)

where D2(0,x,y)_a indicates backprojection data for the view βa, and D2(0,x,y)_b indicates backprojection data for the view βb. Incidentally, the sum of the con beam reconstruction weight coefficients corresponding to the beams opposite to each other is given as follows (Equation 13):

ωa+ωb=1 (Equation 13)

This additional weight process has effect to reduce a cone angle archfact.

For instance, the cone beam reconstruction weight coefficients ωa and ωb can make use of ones obtained by the following equations. Incidentally, ga indicates a weight coefficient of the view βa, and gb indicates a weight coefficient of the view βb, respectively. Assuming that ½ of a fan beam angle is γmax, the following equations (Equation 14 to Equation 19) are established.

[10]

ga=f(γmax, αa, βa) (Equation 14)

gb=f(γmax, αb, βb) (Equation 15)

xa=2·ga^{q}/(ga^{q}+gb^{q}) (Equation 16)

xb=2·gb^{q}/(ga^{q}+gb^{q}) (Equation 17)

wa=xa²·(3−2xa) (Equation 18)

wb=xb²·(3−2xb) (Equation 19)

(For instance, q=1)

Assuming that as examples of ga and gb, max[ ] are defined as functions that take large values, the following equations (Equation 20 and Equation 21) are given as follows:

[11]

ga=max[0, {(π/2+γmax)−|βa|}]·|tan(αa)| (Equation 20)

gb=max[0, {(π/2+γmax)−|βb|}]·|tan(αb)| (Equation 21)

In the case of the fan beam image reconstruction, each pixel on the reconstruction area P is further multiplied by a distance coefficient. Assuming that the distance from the focal point of the X-ray tube 21 to each of the detector row j and channel i of the multi-row X-ray detector 24 is r0, and the distance from the focal point of the X-ray tube 21 to each pixel on the reconstruction area P is r1, the distance coefficient is given as (r1/r0)². In the case of parallel beam image reconstruction, each pixel on the reconstruction area P may be multiplied by the cone beam reconstruction weight coefficient w(i, j) alone.

At Step S63, the projection data D2(view, x, y) is added to its corresponding backprojection data D3(x, y) cleared in advance in association with each pixel. The flowchart for the three-dimensional backprojection process of FIG. 7 is equivalent to one in which the image reconstruction area P shown in FIG. 8 is described as a square of 512×512 pixels.

<Contrast Agent Synchronous Imaging>

Embodiments of contrast agent synchronous imaging will be shown below using the image reconstruction for the tomographic image shown above.

### First Embodiment

It should however be noted that since the contrast agent becomes a significant burden on the subject, the amount of the contrast agent is reduced as much as possible to perform the photography. Therefore, there is a demand to inject the contrast agent into the subject with suitable timing and perform X-ray CT imaging at the timing with a suitable delay time.

The following are known as a method for recognizing this contrast-agent injection and the suitable timing

1. Bolus Tracking Method

It is a method of monitoring a change in CT value at a region of interest ROI after the injection of a contrast agent and starting an actual scan with the most suitable timing when exceeds an established threshold value.

2. Test Injection Method

Prior to an actual scan, this test is that a pre-scan using a small amount of contrast agent is performed to grasp the rate of the bloodstream. Based on this result, this actual scan determine the optimum timing and proceed photograph, in consideration of the amount of the contrast agent and its injection velocity The following explanation is the bolus tracking method.

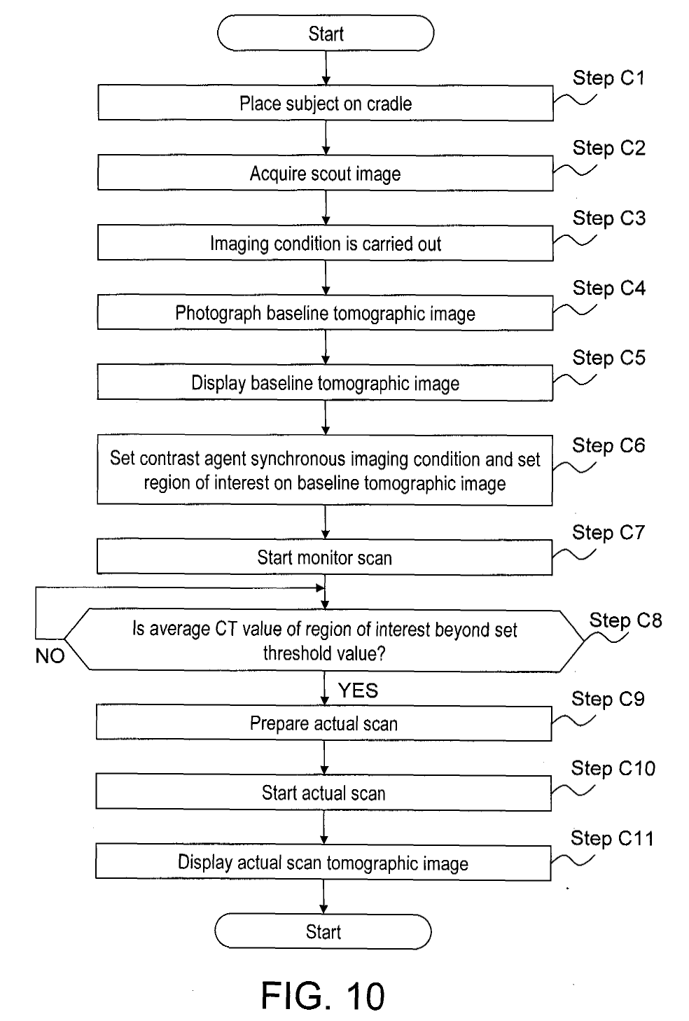

The flow of processing for the contrast agent synchronous imaging is first shown in FIG. 10.

At Step C1, a subject is placed on the cradle 12 and they are aligned with each other. At Step C2, scout image acquisition is performed. At Step C3, an imaging condition setting is carried out.

At Step C4, baseline tomographic image photography is performed. The baseline tomographic image photography is a region of interest ROI employed in each monitor scan MS is set. If a plurality of sheets of tomographic images are photographed in the z-axis direction at the monitor scan MS, then a plurality of sheets of tomographic images are photographed in the z-axis direction even at the baseline tomographic image photography. When a plurality of regions of interest ROI are set in the z-axis direction, a plurality of regions of interest ROI are set in the z-axis direction even at the baseline tomographic image photography. If a plurality of regions of interest ROI are set within a tomographic image in an xy plane, then a plurality of regions of interest ROI are set to within one tomographic image at the baseline tomographic image photography. At Step C5, a baseline tomographic image display CSI is performed. At Step C6, a contrast agent synchronous imaging condition setting is carried out.

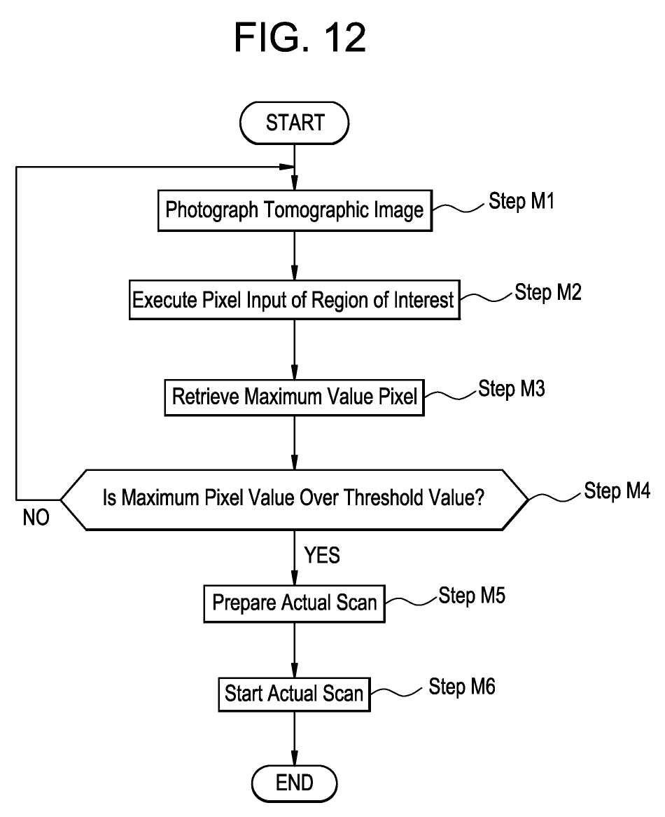

At Step C7, a monitor scan MS is started. At Step C8, it is determined whether an average CT value in the region of interest ROI exceeds a set threshold value. If the answer is found to be YES, then the flow of processing proceeds to Step C9. If the answer is found to be NO, then Step C8 is repeated. The monitor scan will be explained later using FIGS. 12 through 30.

At Step C9, a preparation for an actual scan is made. The cradle 12 on the photographing table 10 is shifted to the position for the actual scan. At Step C10, the actual scan is started. At Step C11, an actual scan tomographic image display is carried out.

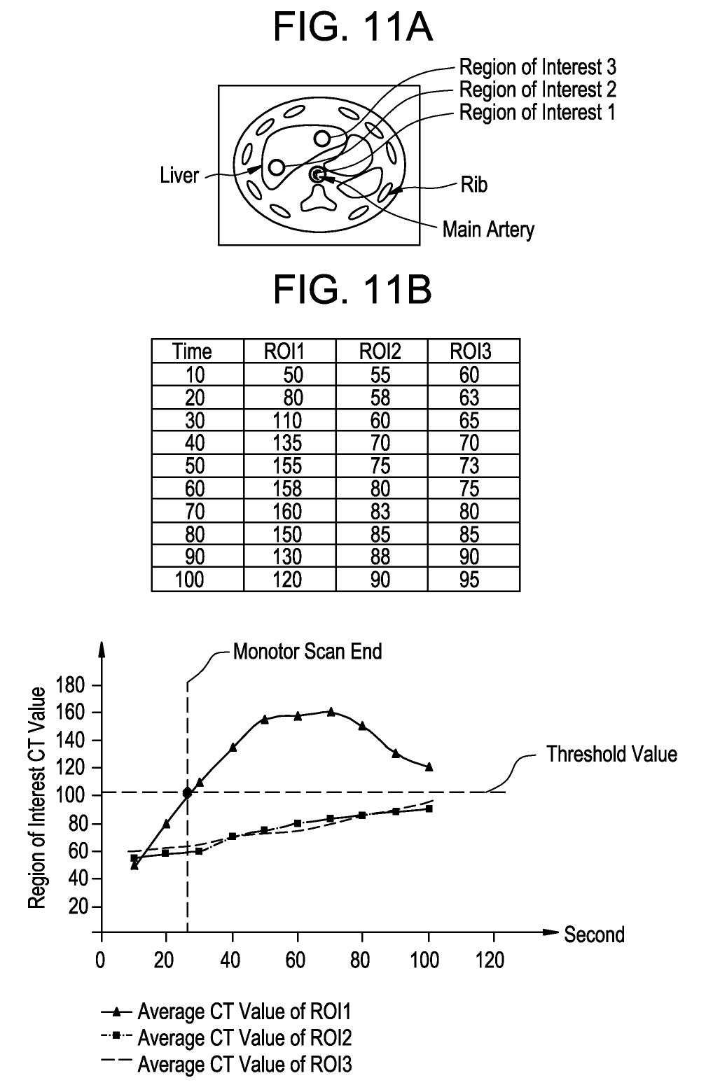

FIG. 11A is a diagram showing a baseline tomographic image, and FIG. 11B is a table and a graph showing display examples of monitor scans MS for the contrast agent synchronous imaging.

In the baseline tomographic image shown in FIG. 11A, a region of interest ROI1 is set to a main artery. This means that the region of interest is first set to such a main artery that a CT value increases, thereby using it as a trigger for the actual scan. The regions of interest are set to respective portions or regions of a liver as a region of interest ROI2 and a region of interest ROI3. In the graph shown in FIG. 11B, the horizontal axis is defined as a time t, and the vertical axis is defined as a CT value.

The graph of in FIG. 11B shows that a threshold value used as for a trigger for the actual scan is set to the region of interest ROI1 as a CT value 100, and the region of interest ROI1 reaches a predetermined threshold value for a little less than about 30 seconds after the beginning of contrast, so that the actual scan is triggered or started.

<Various Monitor Scans MS>

As to the CT-value confirmation at the monitor scan MS of Step C7 and the regions of interest ROI at Step C8 shown in FIG. 10, there are provided forms of six monitor scans MS shown below.

Monitor Scan MS1

When the pixel having the maximum value exceeds a given threshold value at one region of interest ROI lying within a sheet of tomographic image, an actual scan is started.

Monitor Scan MS2

When the average value of N pixels selected in decreasing order from the maximum value exceeds a given threshold value at one region of interest ROI lying within one tomographic image, the actual scan is started (where N: integer greater than or equal to 2).

Monitor Scan MS3

When the average value of pixels in a two-dimensional continuous region including the maximum value exceeds a given threshold value at one region of interest ROI lying within a sheet of tomographic image, the actual scan is started.

Monitor Scan MS4

When the average value of pixels in a three-dimensional continuous region including the maximum value exceeds a given threshold value at one region of interest ROI in a three-dimensional area within a plurality of sheets of tomographic images, the actual scan is started.

Monitor Scan MS5

When the pixels at the respective regions of interest ROI satisfy the conditions for the CT values at the regions of interest ROI located at plural points within the corresponding tomographic image, the actual scan is started.

Monitor Scan MS6

When the pixels at regions of interest ROI lying in respective tomographic images provided in plural form as viewed in the z-axis direction sequentially satisfy the conditions for the CT values, the actual scan is started. The respective monitor scans will be described below in detail.

<<Embodiment of Monitor Scan MS1>>

A flowchart related to the embodiment of the monitor scan MS1 is shown in FIG. 12. In FIG. 14, a tomographic image CSI is shown on the left, and scanning in a region of interest ROI lying in the tomographic image CSI is shown on the right.

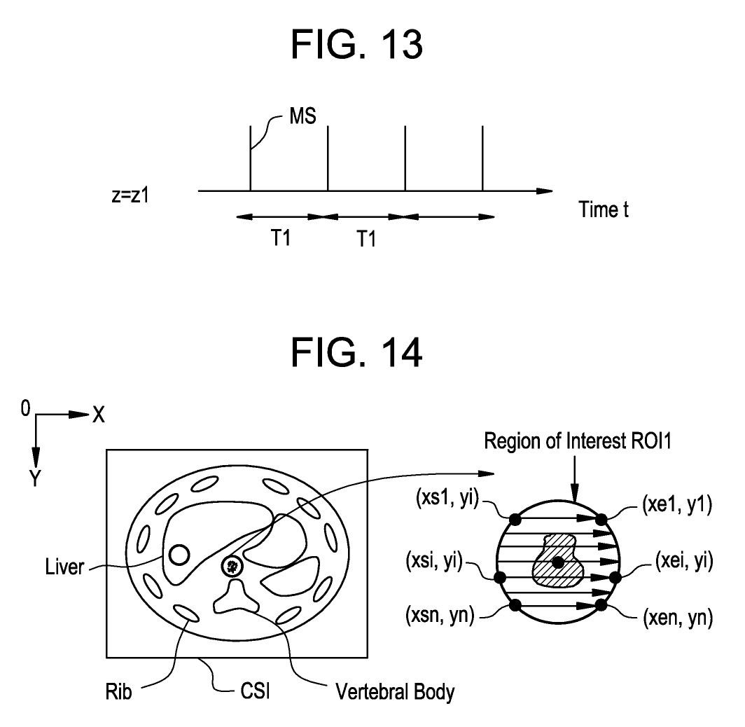

At Step M1, tomographic image photography is performed. Upon the tomographic image photography at the normal monitor scan, continuous imaging is not performed because of a reduction in radiation exposure, and a conventional scan (axial scan) is performed at predetermined time intervals T1 as shown in FIG. 13.

At Step M2, a pixel input at the region of interest ROI is made. Described specifically, as to the pixel input for the region of interest ROI, a CT value of each pixel corresponding to the region of interest ROI is read from a memory storing the corresponding tomographic image therein or an image file for the tomographic image.

At Step M3, each pixel lying in a region of interest ROI1 is scanned as shown in FIG. 14, and the retrieval of the maximum value pixel is performed.

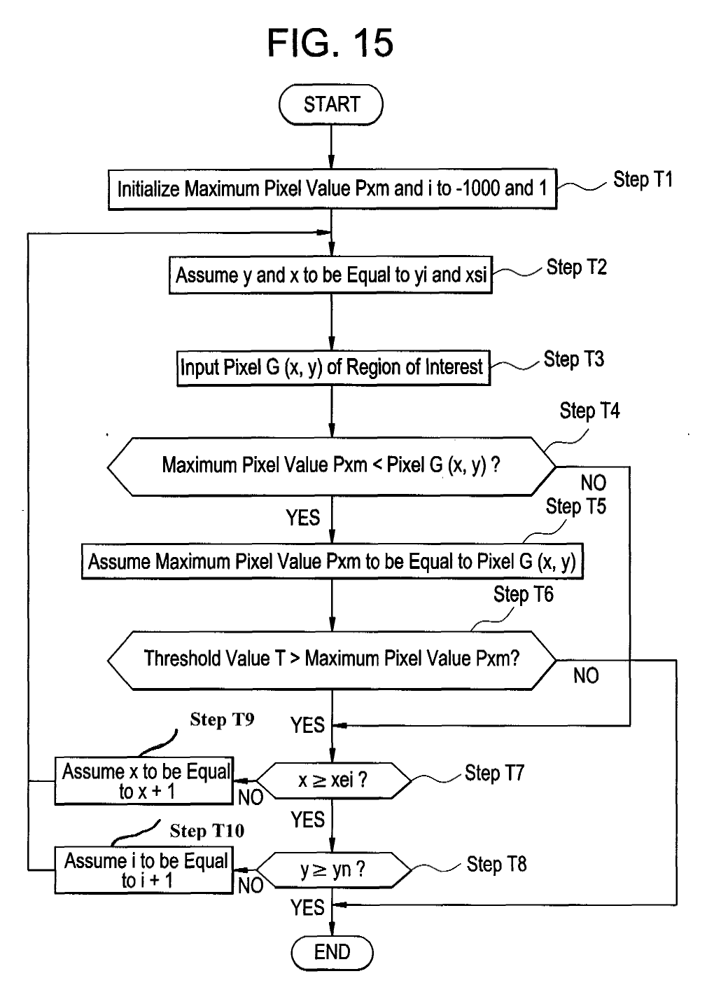

At Step M4, it is determined whether the maximum pixel value exceeds a predetermined threshold value. If the answer is found to be YES, then the flowchart of FIG. 12 proceeds to Step M5. If the answer is found to be NO, then the flowchart returns to Step M1. The details of the flow for the processing from Step M2 to Step M4 will be explained with reference to the flowchart of FIG. 15. Upon the retrieval of the maximum value pixel and the comparison between the maximum value pixel and the threshold value at Steps M2, M3 and M4, the maximum value pixel can be found where the scanning of all pixels in the corresponding region of interest ROI is performed as in the flowchart shown in FIG. 15.

At Step M5, an actual scan preparation is made. The preparation for the actual scan is made up to a scan position for the actual scan. Incidentally, a run-up distance necessary at a helical scan is unnecessary upon an actual scan using a helical shuttle scan and a variable pitch helical scan.

At Step M6, actual scan startup is performed. This is basically identical to Step C10 of FIG. 10.

Incidentally, as to the scanning of all pixels lying in the region of interest ROI, the pixels lying only within the region of interest ROI are scanned as shown in FIG. 14 at the monitor scan MS1. For instance, a range x ε[xsi, xei] between a start point and an end point in an x direction with respect to the pixels in the region of interest ROI at each coordinate of y=yi is scanned. A range y ε[yl, yn] between a start point and an end point in a y direction is scanned in the y direction.

FIG. 15 is a flowchart for describing the retrieval of the maximum pixel value and a comparison between the maximum pixel value and a threshold value. This flowchart will be explained.

At Step T1, the maximum pixel value Pxm and i are respectively initialized to −1000 and 1.

At Step T2, y=yi and x=xsi.

At Step T3, a pixel G (x, y) at a region of interest ROI is inputted.

At Step T4, a decision is made as to whether the maximum pixel value Pxm<G (x, y). If the answer is found to be YES, then the flowchart proceeds to Step T5. If the answer is found to be NO, then the flowchart proceeds to Step T7.

At Step T5, the maximum pixel value Pxm=G (x, y).

At Step T6, a decision is made as to whether a threshold value T>Pxm. If the answer is found to be YES, then the flowchart proceeds to Step T7. If the answer is found to be NO, then the flowchart is completed under the result that the maximum pixel value Pxm has been brought to greater than the threshold value T.

At Step T7, a decision is made as to whether x≧xei. If the answer is found to be YES, then the flowchart proceeds to Step T8. If the answer is found to be NO, then the flowchart proceeds to Step T9.

At Step T8, a decision is made as to whether y≧yn. If the answer is found to be YES, then the flowchart is completed. If the answer is found to be NO, then the flowchart proceeds to Step T10.

At Step T9, x=x+1. Thus, an x coordinate of each pixel in the region of interest ROI is advanced to the following x coordinate, and the flowchart returns to Step T2.

At Step T10, i=i+1. Thus, a y coordinate of each pixel in the region of interest ROI is advanced to the following y coordinate, and the flowchart returns to Step T2.

It is possible that the searching process to find out the maximum pixel values for all the pixels in the region of interest ROI in the above-described manner. Whether the maximum pixel value has exceeded the predetermined threshold value can be understood.

The region of interest ROI set at each monitor scan MS for the contrast agent synchronous imaging is set to blood vessels such as a main artery in which the contrast agent flows and each CT value rises. When the CT value in the blood vessel based on the contrast agent exceeds a certain predetermined threshold value set in advance, the flowchart proceeds to Step C9 and Step C10 of FIG. 10, where the actual scan for the contrast agent synchronous imaging is started.

The average CT value in each region of interest ROI at the monitor scan MS has heretofore been used. There was, however, a case in which a trigger could not be applied well due to the fact that the CT value did not rise sufficiently where the region of interest ROI was excessively large with respect to the diameter of the blood vessel, and the like. In order to avoid it, the actual scan may be started when the CT value of the maximum pixel value in each region of interest ROI exceeds the threshold value. In this case, the trigger for the actual scan can be applied stably regardless the size of each region of interest ROI.

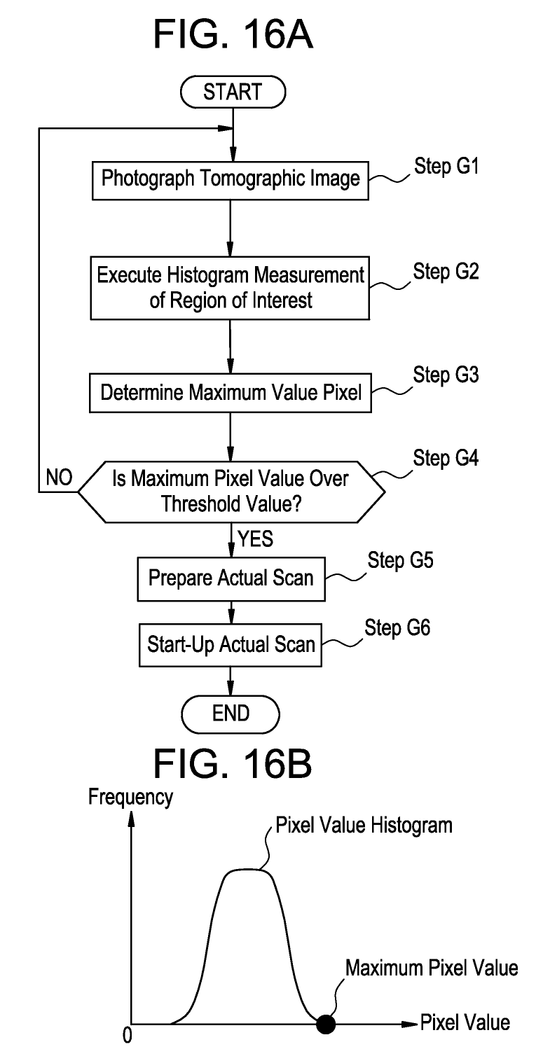

FIG. 16A is a flowchart for describing image processing of a monitor scan MS1 based on a histogram measurement, and FIG. 16B is a diagram showing the result of the histogram measurement, respectively. In FIG. 16A shows the flow which used histogram measurement method.

At Step G1, tomographic image photography is performed.

At Step G2, a histogram measurement for each region of interest ROI is made.

At Step G3, a maximum value pixel is determined.

At Step G4, it is determined whether the maximum value pixel exceeds a threshold value. If the answer is found to be YES, then the processing flow proceeds to Step G5. If the answer is found to be NO, then the processing flow returns to Step G1. At Step G3, the maximum pixel value is determined from a pixel value (CT value) histogram lying in such a region of interest ROI as shown in FIG. 16B, which has been determined at Step G2. The determination of Step G4 is used the maximum pixel value. Thus, a simple flow of processing can also be made by using the basic image processing.

At Step G5, an actual scan preparation is done. At Step G6, an actual scan is made.

<<Embodiment of Monitor Scan MS2>>

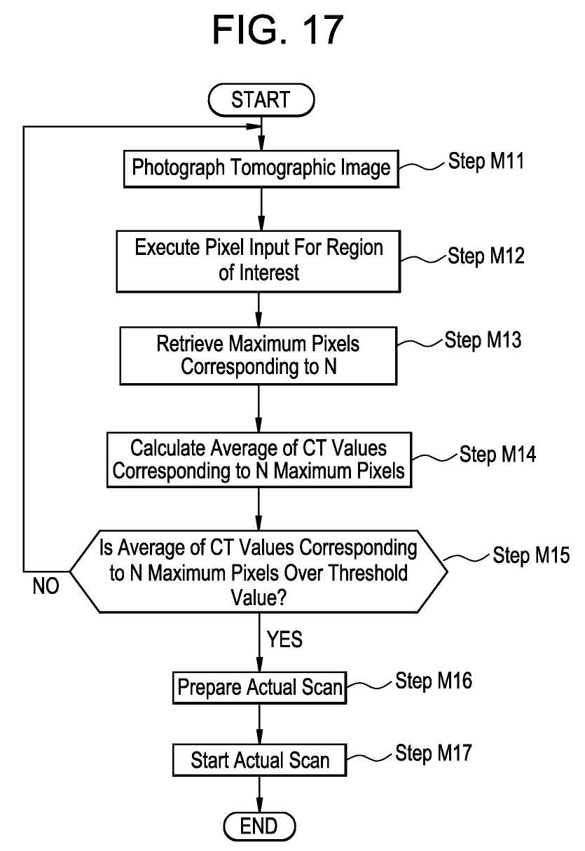

A flowchart related to the monitor scan MS2 is shown in FIG. 17.

At Step M11, tomographic image photography is performed. For this photography, because of a reduction in radiation exposure, the conventional scan (axial scan) is performed at predetermined time intervals T1 as shown in FIG. 13.

At Step M12, a pixel input at a region of interest ROI is made.

At Step M13, each pixel lying in the region of interest ROI is scanned same as shown in FIG. 14, and a retrieval corresponding to the maximum pixels N is performed as to the scanning in the region of interest ROI, same as shown in FIG. 14, a range y ε[yl, yn] between a start point and an end point in a y direction is scanned. At a range x ε[xsi, xei] between a start point and an end point in an x-axis direction is scanned at each y-axis direction coordinate position.

At Step M14, a CT value average calculation corresponding to the maximum pixels N is performed.

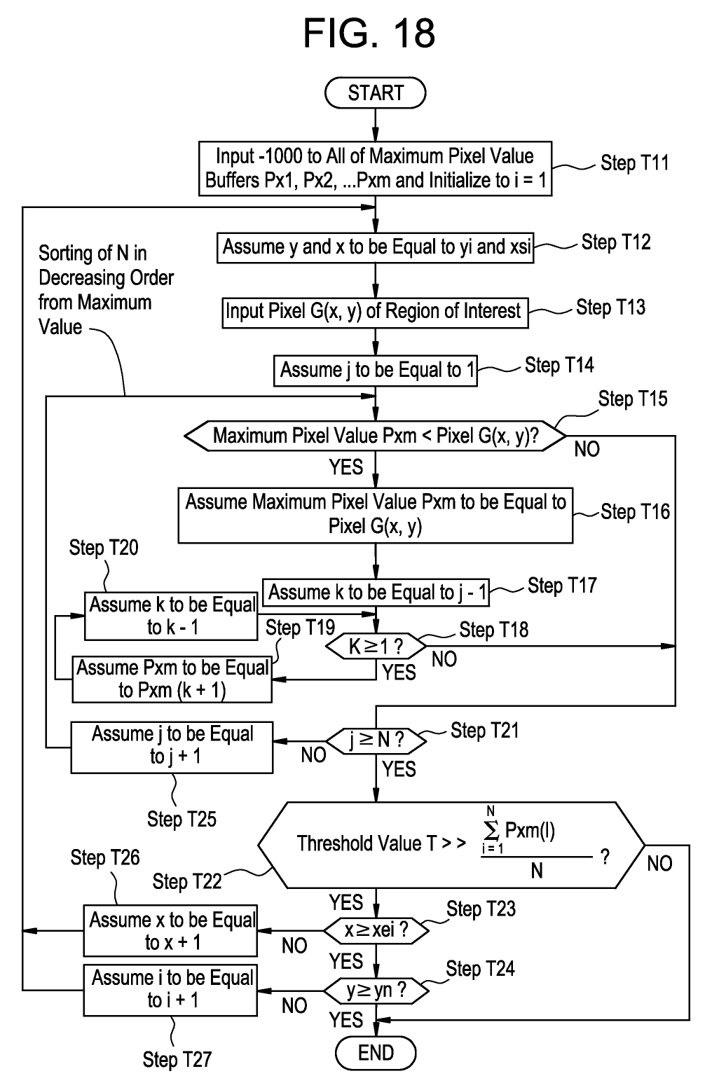

At Step M15, it is determined whether the average of CT values corresponding to the maximum pixels N exceeds a certain predetermined threshold value. If the answer is found to be YES, then the flowchart proceeds to Step M16. If the answer is found to be NO, then the flowchart returns to Step M11. The details of the flow of processing from Step M12 to Step M14 will be explained by reference to a flowchart shown in FIG. 18. Upon a comparison as to whether the average value of N pixels selected in decreasing order from the maximum pixel value at Step M12, Step M13 and Step M14 exceeds a threshold value, the scanning of all the pixels in the region of interest ROI is performed as in the flowchart of FIG. 18. Consequently, the average value of N pixels selected in decreasing order from the maximum pixel value is determined.

At Step M16, an actual scan preparation is made. At Step M17, an actual scan is performed.

FIG. 18 is a flowchart showing the inspection of N maximum pixel values, and comparisons between N maximum pixel values and a threshold value. This flowchart will be explained.

At Step T11, −1000 is inputted to all of maximum pixel value buffers Pxm (1), Pxm (2), . . . Pxm (N), and initialization for i=1 is performed.

At Step T12, y=yi and x=xsi.

At Step T13, a pixel G (x, y) at a region of interest ROI is inputted.

At Step T14, j=1.

At Step T15, it is determined whether Pxm (j)<G (x, y). If the answer is found to be YES, then the flowchart proceeds to Step T16. If the answer is found to be NO, then the flowchart proceeds to Step T21.

At Step T16, Pxm (j)=G (x, y).

At Step T17, k=j−1.

At Step T18, it is determined whether k≧1. If the answer is found to be YES, then the flowchart proceeds to Step T19. If the answer is found to be NO, then the flowchart proceeds to Step T21.

At Step T19, Pxm (k)=Pxm (k+1).

At Step T20, k=k−1 and the flowchart returns to Step T18.

At Step T21, it is judged whether j≧N. If the answer is found to be YES, then the flowchart proceeds to Step T22. If the answer is found to be NO, then the flowchart proceeds to Step T25.

At Step T22,

[12] it is determined whether threshold value

$$T > \frac{{\sum }_{i = 1}^{N} Pxm ( l )}{N} .$$

### If the answer is found to be YES, then the flowchart proceeds to Step T23. If the answer is found to be NO, then the flowchart is completed.

At Step T23, it is determined whether x≧xei. If the answer is found to be YES, then the flowchart proceeds to Step T24. If the answer is found to be NO, then the flowchart proceeds to Step T26.

At Step T24, it is determined whether y≧yn. If the answer is found to be YES, then the flowchart is ended. If the answer is found to be NO, then the flowchart proceeds to Step T27.

At Step T25, j=j+1, and the flowchart returns to Step T15.

At Step T26, x=x+1. Thus, an x coordinate of each pixel in the region of interest ROI is advanced to the following y coordinate, and the flowchart returns to Step T12.

At Step T27, i=i+1. Thus, a y coordinate of each pixel in the region of interest ROI is advanced to the following y coordinate, and the flowchart returns to Step T12.

The region of interest ROI set at each monitor scan MS for the contrast agent synchronous imaging is set to blood vessels such as a main artery in which the contrast agent flows and each CT value rises. When the CT value in the blood vessel based on the contrast agent exceeds a certain predetermined threshold value set in advance, the flowchart proceeds to Step C9 and Step C10 of FIG. 10, where the actual scan for the contrast agent synchronous imaging is started.

Since the average of the CT values corresponding to the N maximum pixel values in the region of interest ROI is used, not average of the CT values in the region of interest ROI, in this case, the trigger for the actual scan can be applied stably regardless the size of each region of interest ROI.

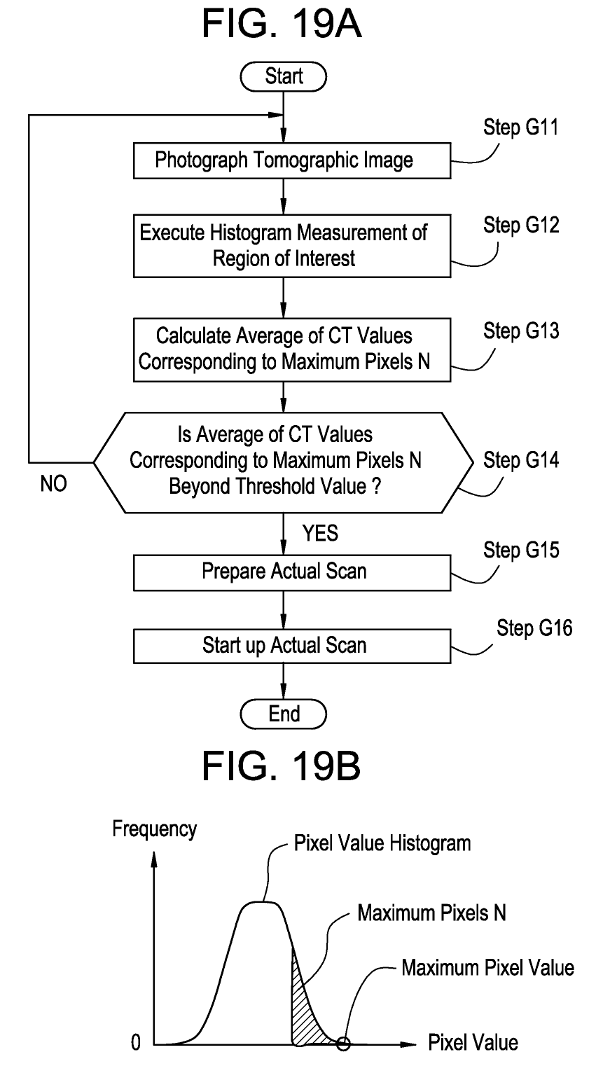

FIG. 19A is a flowchart for describing image processing of a monitor scan MS2 based on a histogram measurement, and FIG. 19B is a diagram showing the result of the histogram measurement, respectively. When the processing of the monitor scan MS2 is realized based on basic image processing, the flow of such processing as shown in FIG. 19A is obtained.

At Step G11, tomographic image photography is performed.

At Step G12, a histogram measurement for each region of interest ROI is made.

At Step G13, a CT value average calculation corresponding to the maximum pixels N is performed. At Step G13, the maximum pixel value and the maximum pixels N are determined from a pixel value (CT value) histogram in such a region of interest ROI as shown in FIG. 19, which has been determined in Step G12. A CT value average corresponding to the maximum pixels N is determined. The CT value average is used in the determination at Step G14. Thus, a simple flow of processing can also be made by using the basic image processing.

At Step G14, it is determined whether the CT value average corresponding to the maximum pixels N exceeds a threshold value. If the answer is found to be YES, then the flowchart proceeds to Step G15. If the answer is found to be NO, then the flowchart returns to Step G11.

At Step G15, an actual scan preparation is done. At Step G16, an actual scan startup is performed. These Steps are respectively identical to Step M5 and Step M6 shown in FIG. 12.

<<Embodiment of Monitor Scan MS3>>

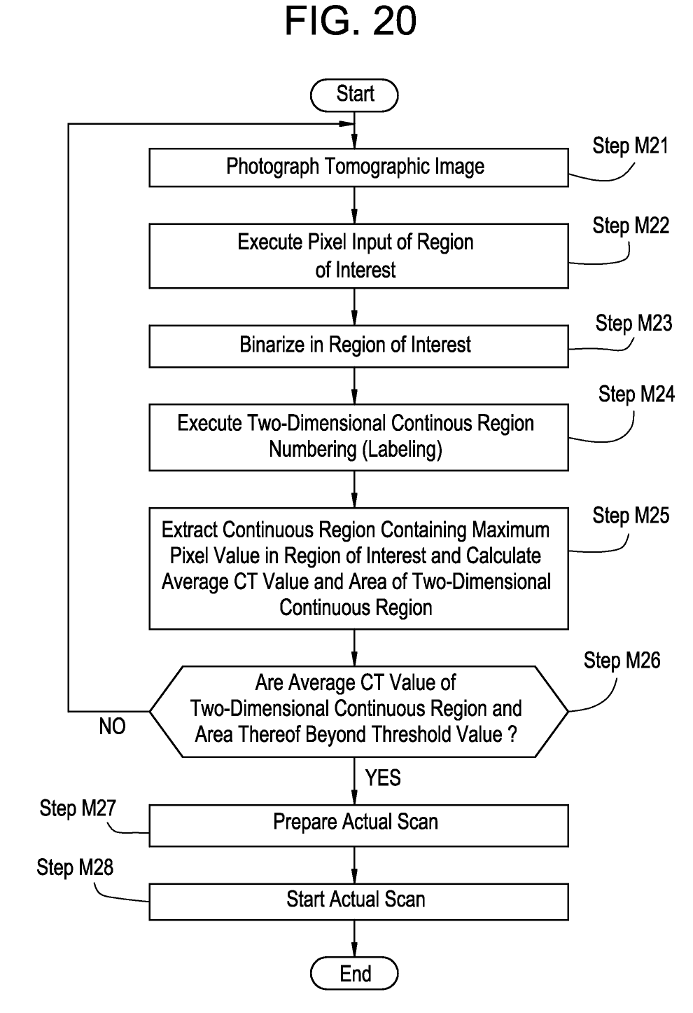

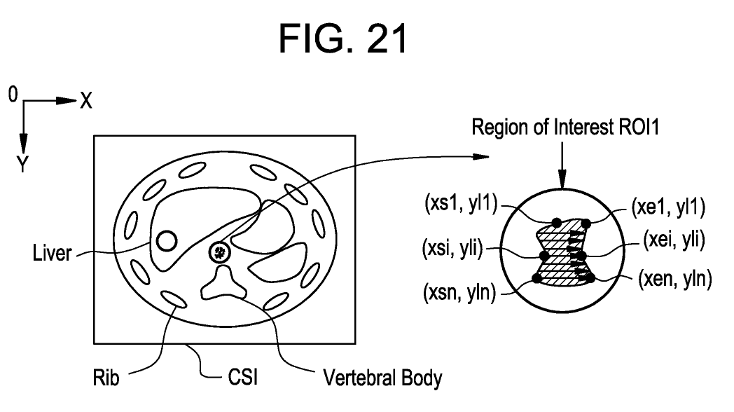

A flowchart illustrative of the monitor scan MS3 is shown in FIG. 20. In FIG. 21, a tomographic image CSI is shown on the left, and scanning in a two-dimensional region of interest ROI lying in the tomographic image CSI is shown on the right.

At Step M21, tomographic image photography is performed. For this photography, because of a reduction in radiation exposure, the conventional scan (axial scan) is performed at predetermined time intervals T1 as shown in FIG. 13.

At Step M22, a pixel input for the region of interest ROI is performed. At Step M23, binarization for the region of interest ROI is performed. At Step M24, two-dimensional continuous region labeling in the region of interest ROI is performed. As to the scanning of all pixels in the region of interest ROI, a range between an x-direction start coordinate and an x-direction end coordinate corresponding to x-direction run coordinates at individual y coordinates, which are obtained by two-dimensional labeling processing as shown in FIG. 21 is scanned. At this time, run coordinates defined by a run-length encoded start point and the length of its line segment or its start and end) That is, two-dimensional continuous region labeling is that a range y ε[yl1, yln] between a start point and an end point in a y direction in the two-dimensional continuous region containing the maximum pixel value is scanned, and a range x ε[xsi, xei] between a start point and an end point in the x direction at each y coordinate is scanned.

At Step M25, in the extracted two-dimensional continuous region, the pixels in the two-dimensional continuous region containing the maximum pixel value are scanned as shown in FIG. 21, and thereby the average CT value of the two-dimensional continuous region and the area thereof are determined.

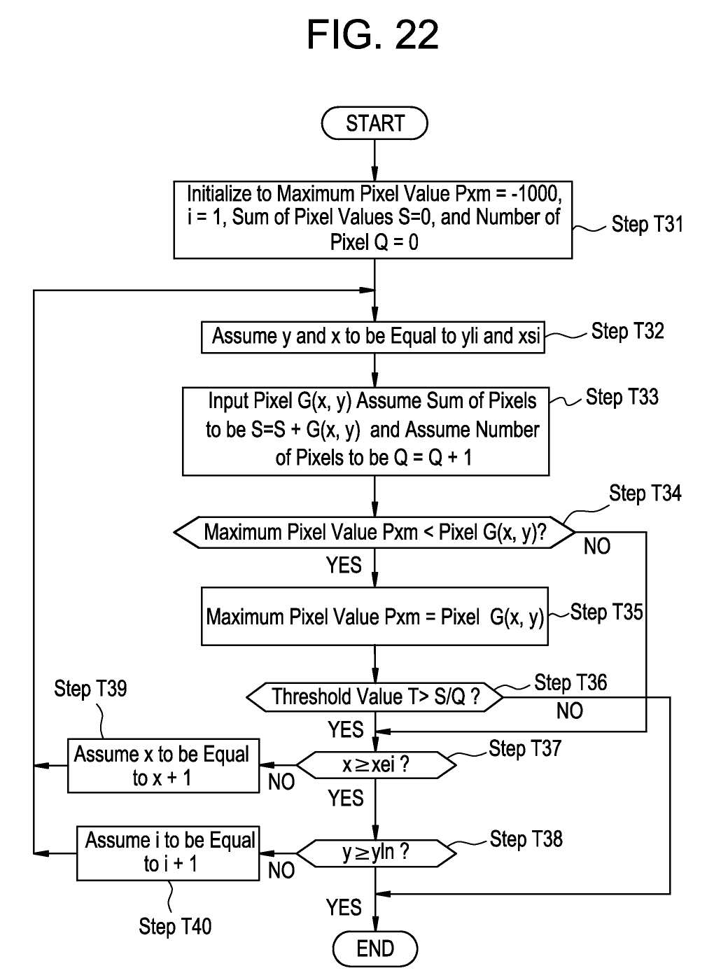

At Step M26, it is determined whether each of the average CT value of the two-dimensional continuous region and the area thereof exceeds a certain predetermined threshold value. If the answer is found to be YES, then the flowchart proceeds to Step M27. If the answer is found to be NO, then the flowchart returns to Step M21. The details of processing from Step M22 to Step M26 will be explained by reference to a flowchart shown in FIG. 22.

At Step M27, an actual scan preparation is carried out, and an actual scan is executed at Step M28.

FIG. 22 is a flowchart for describing the retrieval of the maximum pixel value in the two-dimensional continuous region, and a comparison between the maximum pixel value and a threshold value. This flowchart will be explained.

At Step T31, initialization of the maximum pixel value Pxm=−1000, i=1, the sum of pixel values S=0 and the number of pixels Q=0 is performed.

At Step T32, y=yli and x=xsi.

At Step T33, a pixel G (x, y) for a region of interest ROI is inputted. The sum of pixel values is assumed to be S=S+G (x, y), and the number of pixels is assumed to be Q=Q+1.

At Step T34, it is determined whether the maximum pixel value Pxm<<G (x, y). If the answer is found to be YES, then the flowchart proceeds to Step T35. If the answer is found to be NO, then the flowchart proceeds to Step T37.

At Step T35, the maximum pixel value Pxm is assumed to be Pxm=G(x,y).

At Step T36, it is determined whether a threshold value T>S/Q. If the answer is found to be YES, then the flowchart proceeds to Step T37. If the answer is found to be NO, then the flowchart is completed assuming that the maximum pixel value S/Q has reached greater than the threshold value T.

At Step T37, it is determined whether x≧xei. If the answer is found to be YES, then the flowchart proceeds to Step T38. If the answer is found to be NO, then the flowchart proceeds to Step T39.

At Step T38, it is determined whether y≧yln. If the answer is found to be YES, then the flowchart is completed. If the answer is found to be NO, then the flowchart proceeds to Step T40.

At Step T39, x=x+1. Thus, an x coordinate of each pixel in the region of interest ROI is advanced to the following x coordinate, and the flowchart returns to Step T32.

At Step T40, i=i+1. Thus, a y coordinate of each pixel in the region of interest ROI is advanced to the following y coordinate, and the flowchart returns to Step T32.

The region of interest ROI set at each monitor scan MS for the contrast agent synchronous imaging is set to blood vessels such as a main artery in which the contrast agent flows and each CT value rises. When the CT value in the blood vessel based on the contrast agent exceeds a certain predetermined threshold value set in advance, the flowchart proceeds to Step C9 and Step C10 of FIG. 10, where the actual scan for the contrast agent synchronous imaging is started.

Since the average CT value of the two-dimensional continuous region corresponding to the contrast blood portion is used, not average of the CT values in the region of interest ROI, in this case, the trigger for the actual scan can be applied stably regardless by the size of each region of interest ROI.

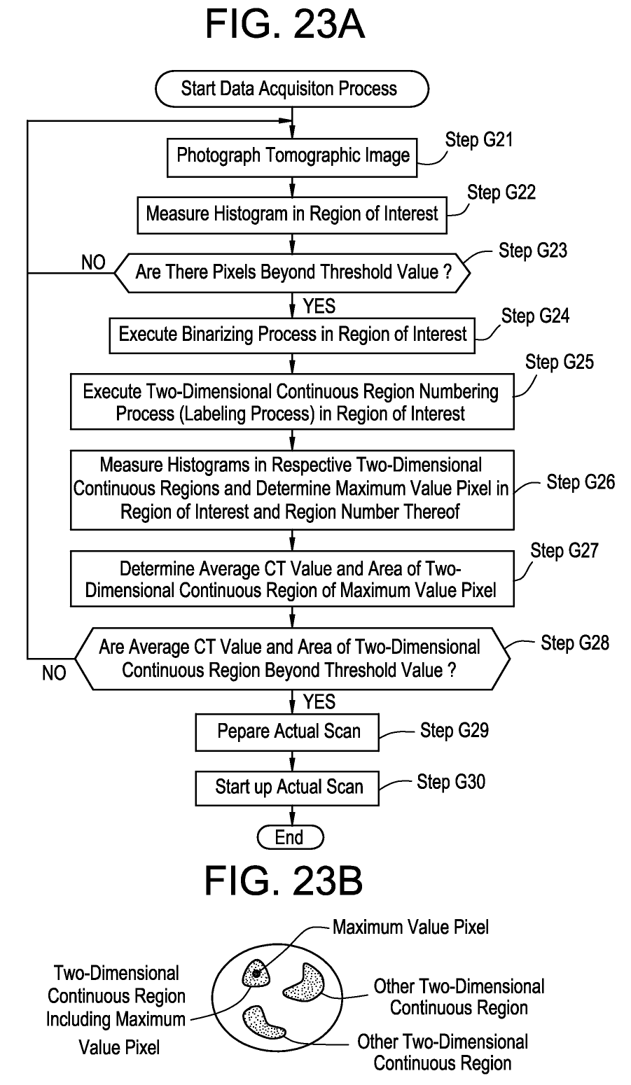

FIG. 23A is a flowchart for describing image processing of a monitor scan MS2 based on a histogram measurement, and FIG. 23B is a diagram showing the maximum pixel and two-dimensional continuous regions. When the processing of the monitor scan MS3 is realized based on basic image processing, the flow of such processing as shown in FIG. 23A is obtained.

At Step G21, tomographic image photography is performed.

At Step G22, a histogram measurement for each region of interest ROI is done.

At Step G23, it is determined whether each pixel exceeding a threshold value exists. If the answer is found to be YES, then the flowchart proceeds to Step G24. If the answer is found to be NO, then the flowchart returns to Step G21.

At Step G24, a binarizing process in the region of interest ROI is performed.

At Step G25, a two-dimensional continuous region numbering process (labeling process) in the region of interest ROI is performed.

At Step G26, a histogram measurement for each two-dimensional continuous region is performed, and the maximum value pixel and its region number are determined.

At Step G27, the average CT value of each two-dimensional continuous region with the maximum value pixel, and the area thereof are determined. At Step G27, the average CT value of the two-dimensional continuous region including the maximum pixel value, and the area of each two-dimensional continuous region are determined as shown in FIG. 23B.

At Step G28, it is determined whether the average CT value of the two-dimensional continuous region exceeds the threshold value. If the answer is found to be YES, then the flowchart proceeds to Step G29. If the answer is found to be NO, then the flowchart returns to Step G21. The binarized threshold value at Step G24 may be set as a threshold value of the average CT value at Step G28.

At Step G29, an actual scan preparation is performed. At Step G30, an actual scan is carried out.

<<Embodiment of Monitor Scan MS4>>

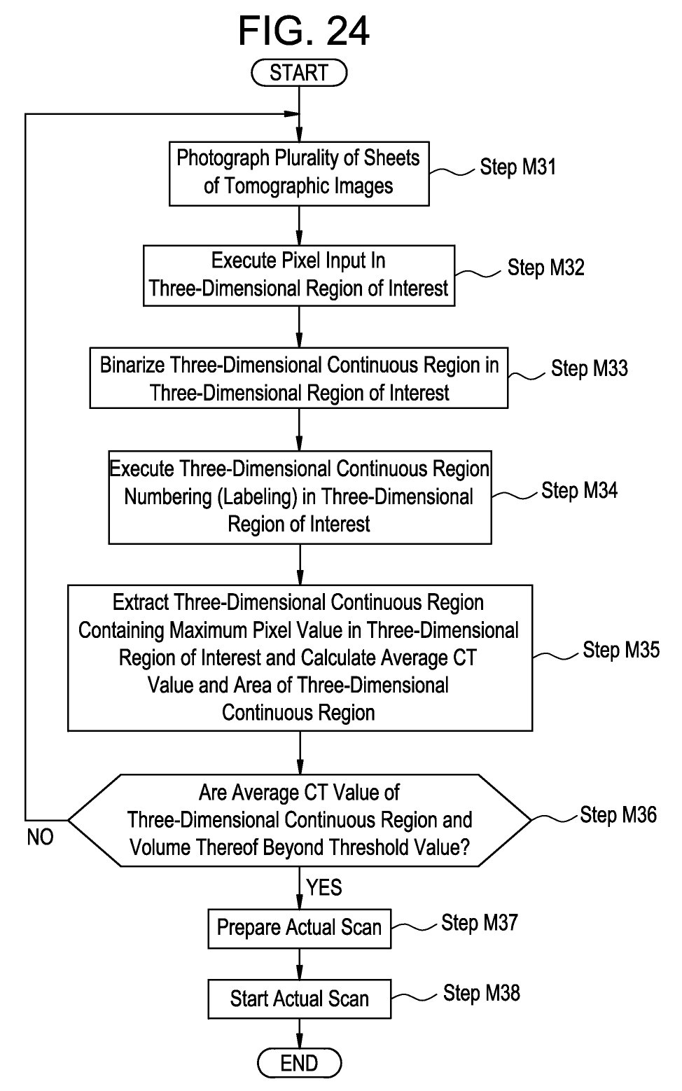

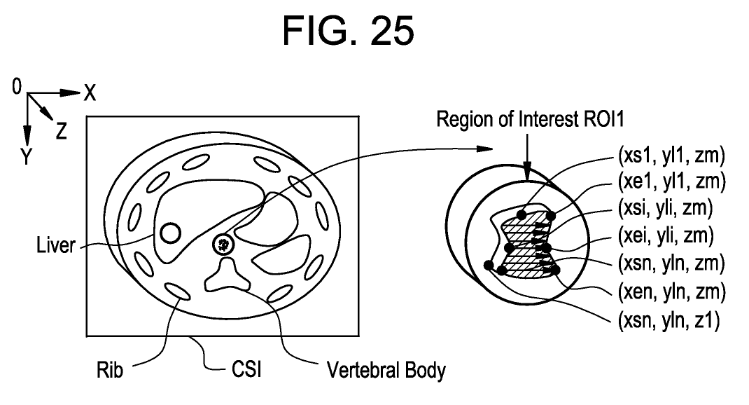

A flowchart illustrative of the monitor scan MS4 is shown in FIG. 24. In FIG. 25, a tomographic image CSI is shown on the left, and the scanning in a three-dimensional region of interest ROI lying in the tomographic image CSI is shown on the right.

At Step M31, a plurality of sheets of tomographic images are photographed. For this photography, because of a reduction in radiation exposure, the conventional scan (axial scan) is performed at predetermined time intervals T1 as shown in FIG. 13.

At Step M32, a pixel input for the three-dimensional region of interest ROI is performed.

At Step M33, binarization for a three-dimensional continuous region in the three-dimensional region of interest ROI is performed.

At Step M34, three-dimensional continuous region labeling in the three-dimensional region of interest ROI is performed. As to the scanning of all pixels in the region of interest ROI at this time, a range between an x-direction start coordinate and an x-direction end coordinate corresponding to x-direction run coordinates at individual y coordinates on each xy plane continuously arranged in a z-axis direction, as shown in FIG. 21, is scanned. That is, at a range z ε[zl, zm] between a start point and an end point in the z-axis direction, a range yε[yl1, yln] between a start point and an end point in a y direction on each xy plane containing the maximum pixel value is scanned, and a range x ε[xsi, xei] between a start point and an end point in the corresponding x direction at each y coordinate is scanned.

At Step M35, the extraction of the three-dimensional continuous region containing the maximum pixel value in the three-dimensional region of interest ROI, and the calculation of both each average CT value of the three-dimensional continuous region and the volume thereof are performed.

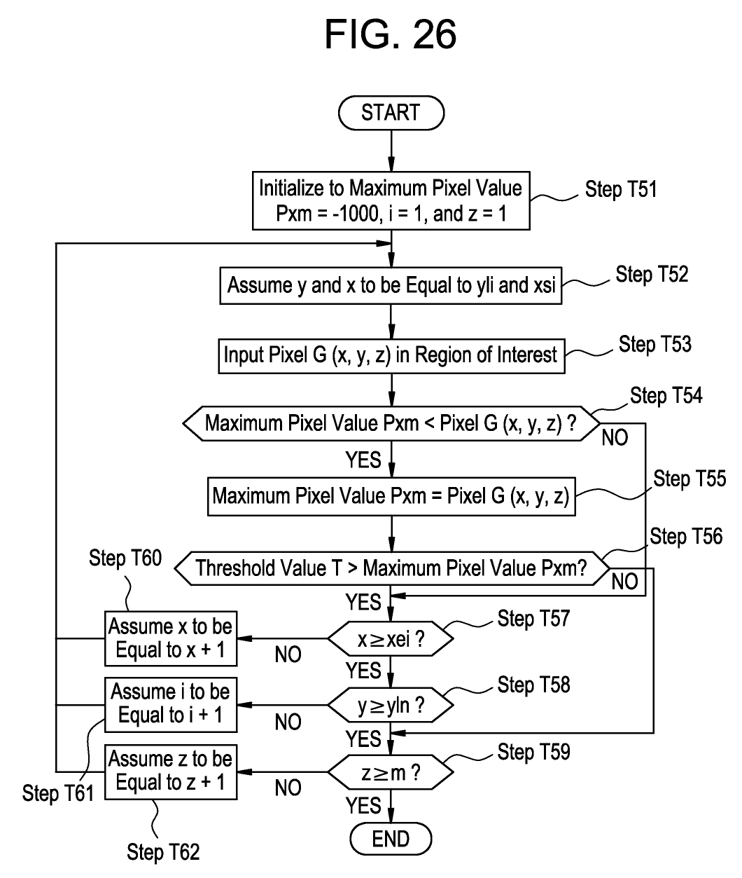

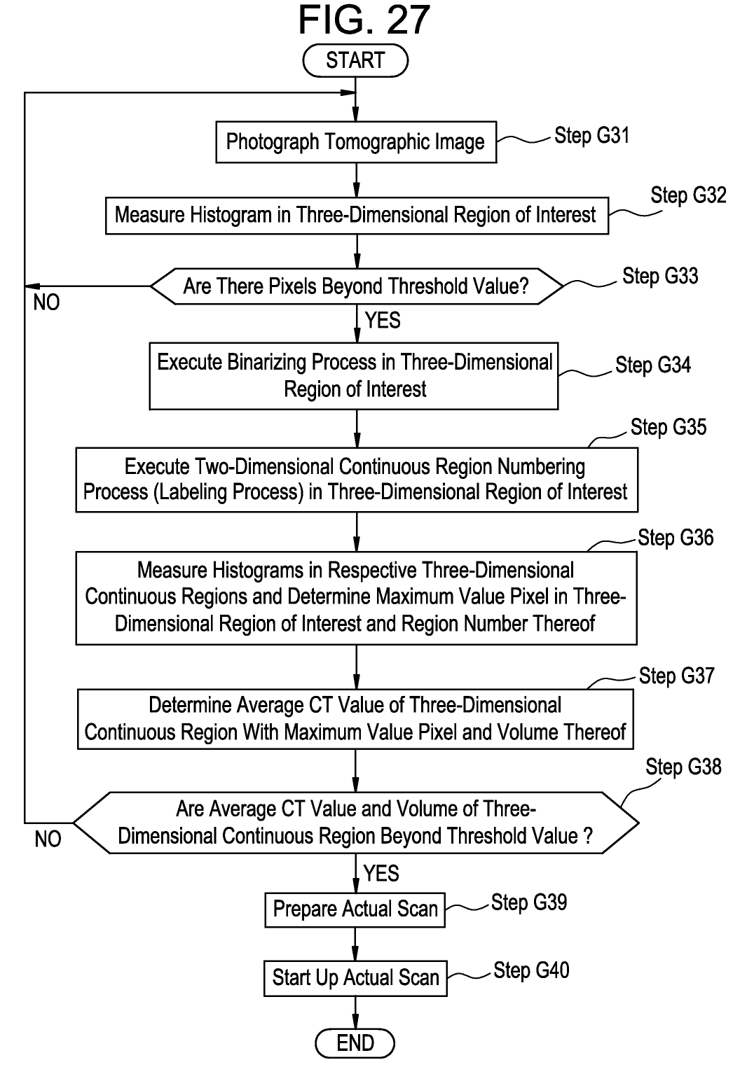

At Step M36, it is determined whether each of the average CT value of the three-dimensional continuous region and the volume thereof exceeds a certain predetermined threshold value. If the answer is found to be YES, then the flowchart proceeds to Step M37. If the answer is found to be NO, then the flowchart returns to Step M31. The details of the flow of processing from Step M32 to Step M36 will be explained by reference to a flowchart shown in FIG. 26.

At Step M37, an actual scan preparation is carried out. An actual scan is executed at Step M38.

FIG. 26 is a flowchart for describing the retrieval of the maximum pixel value in the three-dimensional continuous region shown in FIG. 25, and a comparison between the maximum pixel value and a threshold value. This flowchart will be explained.

At Step T51, initialization of the maximum pixel value Pxm=−1000, i=1, and z=1 is performed.

At Step T52, y=yli and x=xsi.

At Step T53, a pixel G (x, y, z) for a region of interest ROI is inputted.

At Step T54, it is determined whether the maximum pixel value Pxm<<G (x, y, z). If the answer is found to be YES, then the flowchart proceeds to Step T55. If the answer is found to be NO, then the flowchart proceeds to Step T57.

At Step T55, the maximum pixel value Pxm is assumed to be Pxm=G (x, y, z).

At Step T56, it is determined whether a threshold value T>Pxm. If the answer is found to be YES, then the flowchart proceeds to Step T57. If the answer is found to be NO, then the flowchart is completed assuming that the maximum pixel value Pxm has reached greater than the threshold value T.

At Step T57, it is determined whether x≧xei. If the answer is found to be YES, then the flowchart proceeds to Step T58. If the answer is found to be NO, then the flowchart proceeds to Step T60.

At Step T58, it is determined whether y≧yln. If the answer is found to be YES, then the flowchart proceeds to Step T59. If the answer is found to be NO, then the flowchart proceeds to Step T61.

At Step T59, it is determined whether z≧m. If the answer is found to be YES, then the flowchart is completed. If the answer is found to be NO, then the flowchart proceeds to Step T62.

At Step T60, x=x+1. Thus, an x coordinate of each pixel in the region of interest ROI is advanced to the following x coordinate, and the flowchart returns to Step T52.

At Step T61, i=i+1. Thus, a y coordinate of each pixel in the region of interest ROI is advanced to the following y coordinate, and the flowchart returns to Step T52.

At Step T62, z=z+1. Thus, a z coordinate of each pixel in the region of interest ROI is advanced to the following z coordinate, and the flowchart returns to Step T52.

The region of interest ROI set at each monitor scan MS for the contrast agent synchronous imaging is set to blood vessels such as a main artery in which the contrast agent flows and each CT value rises. When the CT value in the blood vessel based on the contrast agent exceeds a certain predetermined threshold value set in advance, the flowchart proceeds to Step C9 and Step C10 of FIG. 10, where the actual scan for the contrast agent synchronous imaging is started.

Since the average CT value of the three-dimensional continuous region corresponding to the blood portion is used, not average of the CT values in the region of interest ROI in this case, the trigger for the actual scan can be applied stably regardless the size of each region of interest ROI.

When the processing of the monitor scan MS4 is realized based on basic image processing, the flow of such processing as shown in FIG. 27 is obtained.

At Step G31, tomographic image photography is performed.

At Step G32, a histogram measurement for each three-dimensional region of interest ROI is done.

At Step G33, it is determined whether each pixel exceeding a threshold value exists. If the answer is found to be YES, then the flowchart proceeds to Step G34. If the answer is found to be NO, then the flowchart returns to Step G31.

At Step G34, a binarizing process in the three-dimensional region of interest ROI is performed. Incidentally, the binarized or digitized threshold value at Step G34 may be set as a threshold value of an average CT value at Step G38.

At Step G35, a three-dimensional continuous region numbering process (labeling process) in the three-dimensional region of interest ROI is performed.

At Step G36, a histogram measurement for each three-dimensional continuous region is performed, and the maximum value pixel and its region number in the three-dimensional region of interest ROI are determined.

At Step G37, the average CT value of each three-dimensional continuous region with the maximum value pixel, and the volume thereof are determined. At Step G37, the average CT value of the three-dimensional continuous region including the maximum value pixel, and the volume of each three-dimensional continuous region are determined.

At Step G38, it is determined whether the average CT value of the three-dimensional continuous region and its volume exceed the threshold value. If the answer is found to be YES, then the flowchart proceeds to Step G39. If the answer is found to be NO, then the flowchart returns to Step G31. The binarized threshold value at Step G34 may be set as a threshold value of the average CT value at Step G38.

At Step G39, an actual scan preparation is performed. At Step G40, an actual scan startup is carried out. These are respectively identical to Step M5 and Step M6 shown in FIG. 12.

<<Embodiment of Monitor Scan MS5>>

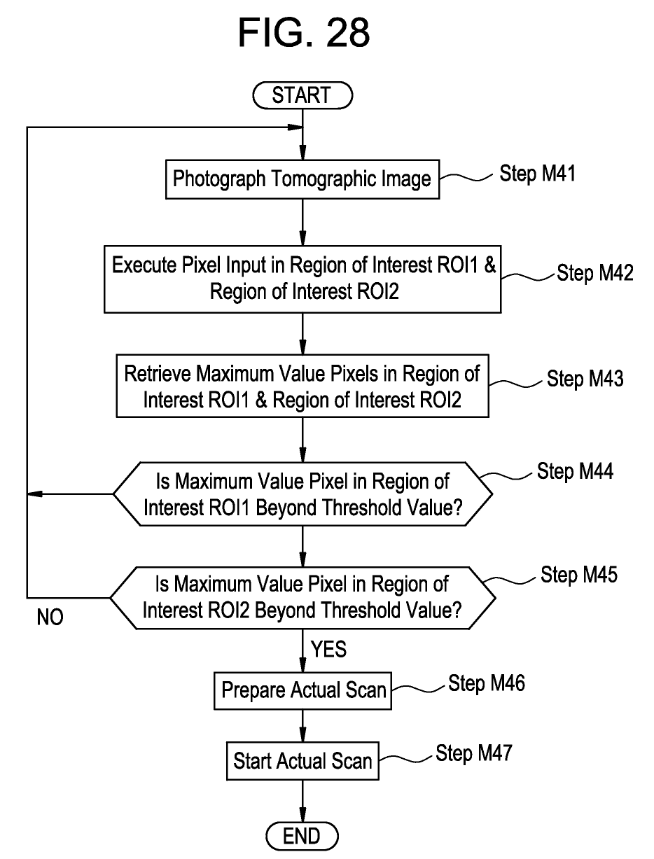

FIG. 28 shows a flowchart for describing the embodiment of the monitor scan MS5.

At Step M41, tomographic image photography is performed. For this photography, because of a reduction in radiation exposure, the conventional scan (axial scan) is performed at predetermined time intervals T1 as shown in FIG. 13

At Step M42, a pixel input for each of a region of interest ROI1 and a region of interest ROI2 is performed. For instance, the region of interest ROI2 corresponds to a portion indicated by a round mark in the liver in FIG. 14.

At Step M43, each pixel in the region of interest ROI is scanned as shown in FIG. 14, and the retrieval of the maximum value pixels in the region of interest ROI1 and the region of interest ROI2 is performed.

At Step M44, it is determined whether the maximum value pixel in the region of interest ROI1 exceeds a given predetermined threshold value. If the answer is found to be YES, then the flowchart proceeds to Step M45. If the answer is found to be NO, then the flowchart returns to Step M41.

At Step M45, it is determined whether the maximum value pixel in the region of interest ROI2 exceeds a certain predetermined threshold value. If the answer is found to be YES, then the flowchart proceeds to Step M46. If the answer is found to be NO, then the flowchart returns to Step M41.

At Step M46, an actual scan preparation is carried out. At Step M47, an actual scan is performed.

In this case, at monitor scan MS, the two regions of interest ROI corresponding to the region of interest ROI1 and the region of interest ROI2 exist in the same xy plane. When plural regions of interest, e.g., two regions of interest ROI are normally set at the monitor scan MS for the contrast agent synchronous imaging, one region of interest ROI is set to blood vessels such as a main artery in which each CT value is likely to rise to begin with. Another region of interest ROI is set to an organ to be subjected to such a portion as to be first stained with the contrast agent. Thus, when the plural regions of interest ROI is set, the actual scan startup can reliably be applied without its missing as compared with the case in which only one region of interest ROI is set. In addition, setting up the plural regions of interest ROI prevent a malfunction that the value of each pixel other than the blood vessel happens to exceed threshold value and the actual scan startup is applied.

<<Embodiment of Monitor Scan MS6>>

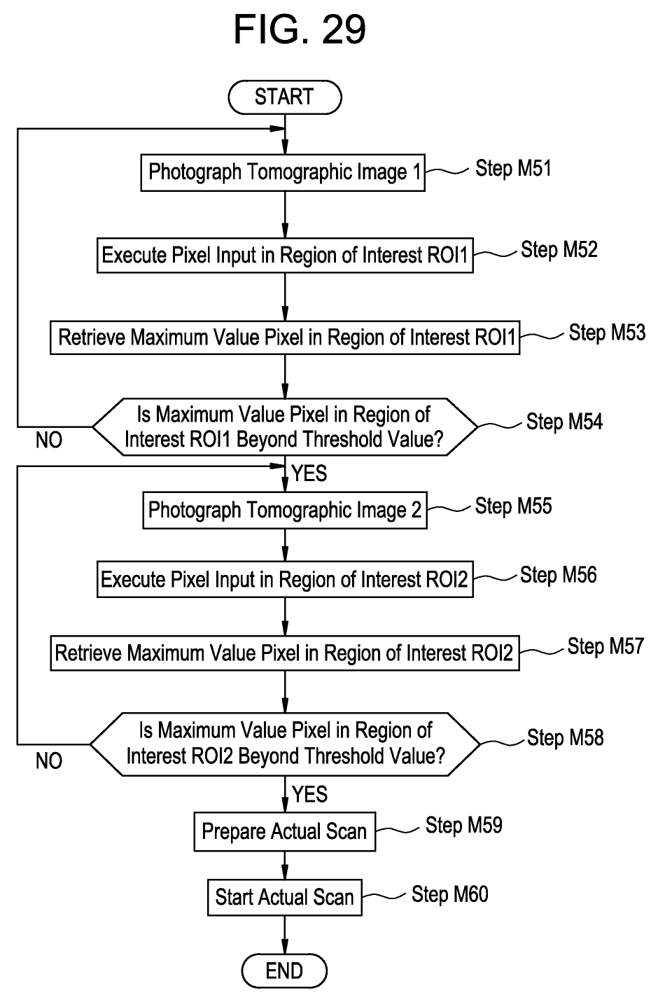

FIG. 29 shows a flowchart for describing the embodiment of the monitor scan MS6.

At Step M51, photography for a tomographic image 1 is performed. For this photography, because of a reduction in radiation exposure, the conventional scan (axial scan) is performed at predetermined time intervals T1 as shown in FIG. 13

At Step M52, a pixel input for a region of interest ROI1 is carried out.

At Step M53, each pixel in the region of interest ROI1 is scanned as shown in FIG. 14, and the retrieval of the maximum value pixel in the region of interest ROI1 is performed.

At Step M54, it is determined whether the maximum value pixel in the region of interest ROI1 exceeds a given predetermined threshold value. If the answer is found to be YES, then the flowchart proceeds to Step M55. If the answer is found to be NO, then the flowchart returns to Step M51.

At Step M55, photography for a tomographic image 2 is performed. Because of a reduction in radiation exposure, the conventional scan (axial scan) is performed at predetermined time intervals T1 as shown in FIG. 13 for this photography as well.

At Step M56, a pixel input for a region of interest ROI2 is performed.

At Step M57, each pixel in the region of interest ROI2 is scanned as shown in FIG. 14, and the retrieval of the maximum value pixel in the region of interest ROI2 is performed.

At Step M58, it is determined whether the maximum value pixel in the region of interest ROI2 exceeds a given predetermined threshold value. If the answer is found to be YES, then the flowchart proceeds to Step M59. If the answer is found to be NO, then the flowchart returns to Step M55.

At Step M59, an actual scan preparation is carried out. At Step M60, an actual scan is done.

In this case, the two monitor scan MS are used. These two are considered as tomographic images at different z-axis coordinate positions or two sheets of tomographic images of a plurality of tomographic images at the same z-axis coordinate position detected by a multi-row X-ray detector. In any case, one or plural regions of interest ROI are respectively set to the two sheets of tomographic images at the positions away in the z-axis direction. When first region of interest ROI is set, it is set to blood vessels such as a main artery in which CT value is likely to rise to begin with. When the plural regions of interest ROI are set, they are further set to a subject organ to be photographed, i.e., such a portion that the contrast agent is likely to be first stained. These regions of interest ROI can be set to their corresponding tomographic images at the coordinate positions different in the z-axis direction.

If the tomographic images are row in the multi-row X-ray detector 24, x-ray data acquisition system remain at given z-axis direction coordinate positions, and the monitor scan MS may be performed at given predetermined time intervals T1 as shown in FIG. 13.

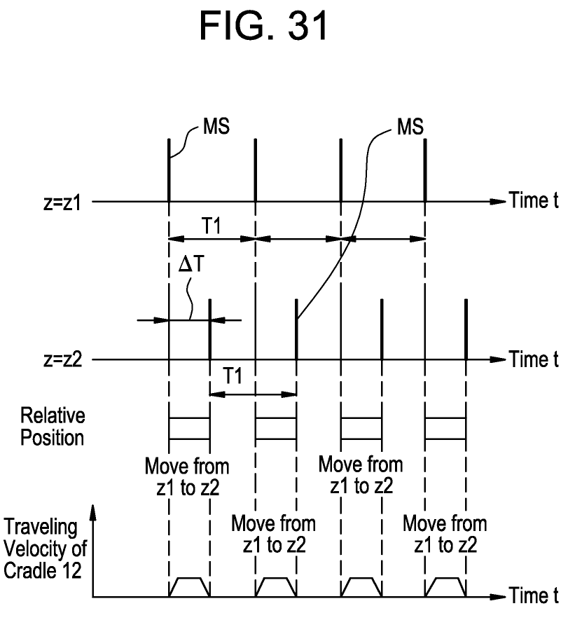

In the case of two tomographic images that exceed the width of the multi-row X-ray detector 24 and are away in the z-axis direction, cradle 12 is moved and two monitor scans MS are executed in the z-axis direction. For example, as shown in FIG. 31, the monitor scans MS first photograph the first sheet of tomographic image at a z-axis direction coordinate position of z=z1 and the cradle 12 are shifted to z-axis direction coordinate position of z=z2, and then photograph the second tomographic image. The actual scan is performed after execute this movement several times. Incidentally, a maximum pixel value searching method may be similar to the embodiment at the monitor scan MS1. When the trigger for the actual scan is applied to the regions of interest ROI placed at the two spots as viewed in the different z-axis coordinate position the trigger for the actual scan can reliably be applied without its missing as compared with the case in which only one region of interest ROI is set on one tomographic image. Although the maximum pixel value of the region of interest ROI is used in this case, a similar or further effect is obtained even when the average of CT values corresponding to the N maximum pixels as in the monitor scan MS2, the average CT value of each two-dimensional continuous region containing the maximum pixel value as in the monitor scan MS3, and the average CT value of the three-dimensional continuous region containing the maximum pixel value in the region of interest ROI as in the monitor scan MS4 are used. Confirming the attainment of the contrast agent at the plural regions of interest ROI in the plural tomographic images in this way makes it possible to prevent a malfunction that the value of each pixel other than the blood vessel happens to exceed a threshold value and the trigger for the actual scan is applied.

<<Embodiment of Monitor Scan MS7>>

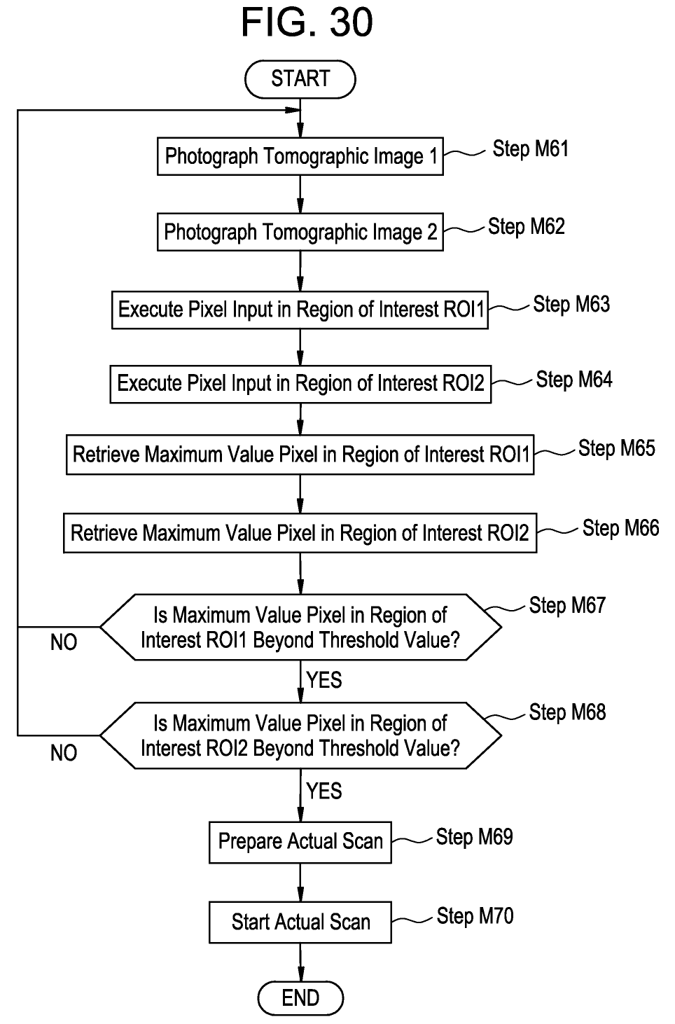

FIG. 30 shows a flowchart for describing the monitor scan MS7.

At Step M61, photography for a tomographic image 1 is performed.

At Step M62, photography for the tomographic image 2 is performed.

Continuous imaging of the tomographic image 1 and the photography of a tomographic image 2 is not performed because of a reduction in radiation exposure, and a monitor scan MS is performed at predetermined time intervals T1 as shown in FIG. 31. Although at this time, the photographing table shuttle between z-axis direction coordinate positions z=z1 and z=z2, T1 and ΔT can be shortened by performing photography or imaging while the photographing table is being accelerated or decelerated as in table travel velocities shown in FIG. 31.

At Step M63, a pixel input for a region of interest ROI1 is performed.

At Step M64, a pixel input for a region of interest ROI2 is performed.

At Step M65, the retrieval of the maximum value pixel in the region of interest ROI1 is carried out.

At Step M66, the retrieval of the maximum value pixel in the region of interest ROI2 is done.

At Step M67, it is determined whether the maximum value pixel in the region of interest ROI1 exceeds a threshold value. If the answer is found to be YES, then the flowchart proceeds to Step M68. If the answer is found to be NO, then the flowchart returns to Step M61.

At Step M68, it is determined whether the maximum value pixel in the region of interest ROI2 exceeds a threshold value. If the answer is found to be YES, then the flowchart proceeds to Step M69. If the answer is found to be NO, then the flowchart returns to Step M61.

At Step M69, an actual scan preparation is done. At Step M70, an actual scan startup is done.

In this case, the two parts of the monitor scan MS are used. There are considered two tomographic images at different z-axis coordinate positions or two sheets of tomographic images of a plurality of tomographic images detected by a multi-row X-ray detector. In any case, one or plural regions of interest ROI are respectively set to the two sheets of tomographic images at the positions away in the z-axis direction. When the first region of interest ROI is set, it is set to blood vessels such as a main artery in which each CT value is likely to rise to begin with. When the plural regions of interest ROI are set, they are further set to a subject organ i.e., such a portion that the contrast agent is likely to be stained. These regions of interest ROI are set to their corresponding tomographic images at the coordinate positions different in the z-axis direction.

If the tomographic images of two parts of monitor scan are different rows in the multi-row X-ray detector 24, x-ray data acquisition remain at given z-axis direction coordinate positions, and the monitor scan MS may be performed at given predetermined time intervals T1 as shown in FIG. 13.

However, two monitor scans MS are executed in the z-axis direction by shifted cradle 12 in the case of two tomographic images that exceed the width of the multi-row X-ray detector and are away in the z-axis direction. As shown in FIG. 31, for example, monitor scan MS photograph the first sheet of tomographic image at a z-axis direction coordinate position of z=z1, and thereafter the cradle 12 are shifted in the z-axis direction, and photograph the second sheet of tomographic image at a z-axis direction coordinate position of z=z2. Assuming that the time necessary to move the relative positions is defined as ΔT as shown in FIG. 31, the tomographic image photography of z=z1 is performed by the monitor scan MS and thereafter the tomographic image photography of z=z2 is performed after the elapse of ΔT seconds. Further, the tomographic image photography of z=z1 is performed after T1−Δ seconds. By repeating it, the tomographic images away from one another at the z-axis direction coordinate position can be photographed at intervals of T1 seconds.

A maximum pixel value retrieval method in this case may be similar to the embodiment at the monitor scan MS1. Setting up the two regions of interest ROI which are away from one another never miss the trigger for the actual scan compared with the case in which only one region of interest ROI is set on one tomographic image. Although the maximum pixel value of each region of interest ROI is used in this case, a similar or further effect is obtained even when the average of CT values corresponding to the N maximum pixels as in the monitor scan MS2, the average CT value of each two-dimensional continuous region containing the maximum pixel value as in the monitor scan MS3, and the average CT value of the three-dimensional continuous region containing the maximum pixel value as in the monitor scan MS4 are used. Confirming the attainment of the contrast agent at the plural regions of interest ROI in the plural tomographic images in this way makes it possible to prevent a malfunction that the value of each pixel other than the blood vessel happens to exceed a given threshold value and the trigger for the actual scan is applied.

By the various embodiments of the monitor scans MS shown above, the trigger for the actual scan can be applied. However, exposure at each monitor scan MS will be explained below.

<<Low Exposure Method at Monitor Scan MS>>

The monitor scan MS is one for originally starting the actual scan with suitable timing and does not contribute to diagnosis directly. Therefore, the exposed dose may preferably be reduced in terms of X-ray exposure. That is, the intervals T1 at the intermittent scans for the monitor scans MS shown in FIGS. 13 and 31 may be preferably longer in terms of the exposed dosage. However, from the point that the actual scan is desired to start at the optimum timing, it is better if the intervals T1 is not rendered so long. Thus, the interval T1 at the intermittent scan for each monitor scan MS has the relationship of a trade-off between the exposed dosage and the optimum timing control.

It is preferable to decrease an X-ray tube current at the monitor scan MS for the purpose of reducing an exposed dosage in terms of a reduction in exposure. However, when the X-ray tube current is excessively reduced, the quality of a tomographic image is deteriorated, and an error occurs upon the measurement of each pixel value in the region of interest ROI. There is a possibility that this measured error will result in an error in timing for starting the actual scan with proper timing. That is, the X-ray tube current has also the relationship of a trade-off between the exposed dosage and the optimum timing control. Therefore, it is preferable to minimize a slice thickness of each tomographic image and minimize an X-ray radiation aperture width too for the purpose of a reduction in X-ray exposure.

On the other hand, upon the actual scan, it is preferable to broad the X-ray radiation aperture width in the z-axis direction as much as possible and carry out the variable pith helical scan and the helical shuttle scan with a fast helical scan pitch in respect that the imaging or photography is performed with a small amount of contrast agent as much as possible to reduce a burden on the subject. Therefore, the X-ray radiation aperture width in the z-axis direction of the monitor scan MS may preferably be narrowed as compared with the actual scan. Therefore, one sheet of tomographic image is enough at a minimum as the tomographic image in the case of the monitor scan MS1, monitor scan MS2, monitor scan MS3 and monitor scan MS5.

It is preferable to control the X-ray collimator 23 in such a manner that upon the monitor scan MS4, the minimum X-ray radiation beam including the three-dimensional region of interest ROI and the minimum X-ray radiation aperture width are reached, thereby to reduce the X-ray exposed dosage at the monitor scan.

In the case of the two tomographic images by the conventional scan (axial scan) or cine scan, corresponding to the z-axis direction coordinate positions at the monitor scan MS6, the slice thickness of each of the respective tomographic images at the respective z-axis direction coordinate positions by the conventional scan (axial scan) or cine scan may preferably be made as thin as possible, and the X-ray radiation aperture width may preferably be narrowed as much as possible.

When the tomographic images at the coordinate positions different in the z-axis direction are set using the two sheets of tomographic images of the plural tomographic images by the multi-row X-ray detector at the monitor scan MS6, bring the two sheets of tomographic images to both ends of the X-ray radiation bean. The X-ray exposed dosage of the monitor scan may preferably be reduced by controlling the X-ray collimator 23 in such a manner that the X-ray radiation aperture width is made as narrow as possible.

<<Actual Scan Preparation and Actual Scan Start>>

The following embodiment is that the operation of the cradle 12, the operations of the X-ray tube 21 and multi-row X-ray detector 24, or the relative operations between the cradle 12 and X-ray tube 21 and the multi-row X-ray detector 24 up to the transition to the actual scan will be explained. The following forms are taken between the actual scan preparation at Step C9 and the actual scan start at Step C10 in FIG. 10.

1. Actual Scan Preparatory Operation 1.

The cradle 12 is moved in the z-axis direction from a monitor scan position and turned back to perform the actual scan. Upon the actual scan, X rays are outputted and thereafter X-ray projection data acquisition is started, and an actual scan z-axis movement is made.

2. Actual Scan Preparatory Operation 2

The cradle 12 is moved in the z-axis direction from the monitor scan position and turned back to perform the actual scan-to acquire X-ray data.

Upon the actual scan, X rays are outputted and thereafter X-ray projection data acquisition is started, and an actual scan z-axis movement is done.

3. Actual Scan Preparatory Operation 3

While the cradle 12 is moved in the z-axis direction from the monitor scan position and turned back to perform the actual scan, it stays in the z-axis direction for a predetermined time interval upon the actual scan and thereafter the actual scan is done.

Upon the actual scan, X rays are outputted and thereafter X-ray projection data acquisition is started, and an actual scan z-axis direction movement is done.

4. Actual Scan Preparatory Operation 4

While the cradle 12 is moved in the z-axis direction from the monitor scan position and the actual scan is performed without its turning back, it stays for a predetermined time interval upon the actual scan, and thereafter the actual scan is done.

Upon the actual scan, X rays are outputted and thereafter X-ray projection data acquisition is started, followed by execution of an actual scan z-axis direction movement.

5. Actual Scan Preparatory Operation 5

The cradle 12 is moved in the z-axis from the monitor scan position and the actual scan is performed.

Upon the actual scan, an actual scan z-axis movement is performed and thereafter X rays are outputted, followed by execution of X-ray projection data acquisition.

6. Actual Scan Preparatory Operation 6

The cradle 12 is moved in the z-axis direction from the monitor scan position, and the actual scan is made without deceleration and acceleration.

<<Actual Scan Preparatory Operation 1>>

The X-ray data acquisition of the actual scan preparatory operation 1 is that the cradle is moved in the z-axis direction from a monitor scan position and turned back to perform an actual scan. Upon the x-ray data acquisition of the actual scan is photographed by the variable pitch helical scan or helical shuttle scan, and relative operations between the cradle 12, X-ray tube 21 and multi-row X-ray detector 24 is performed.

When in this case of X-ray data acquisition, the cradle 12, X-ray tube 21 and multi-row X-ray detector 24 are relatively moved, X-ray projection data acquisition is performed in the z-axis direction from during acceleration. This scan is, since the run-up distance and run-up time and the like for acceleration are unnecessary as the conventional helical scan, X-ray projection data acquisition can be reached in a short period of time.

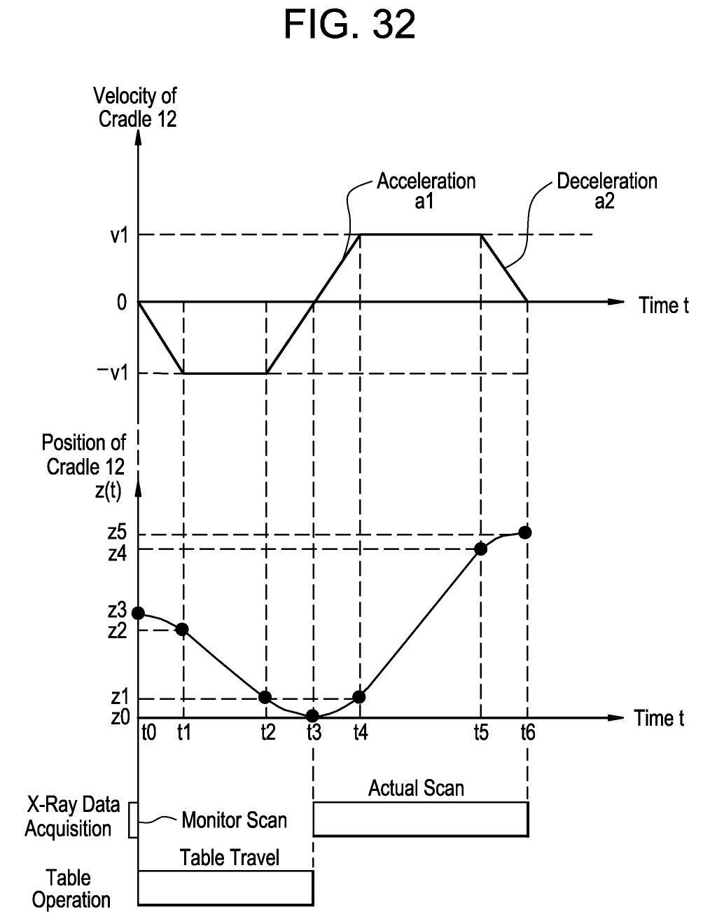

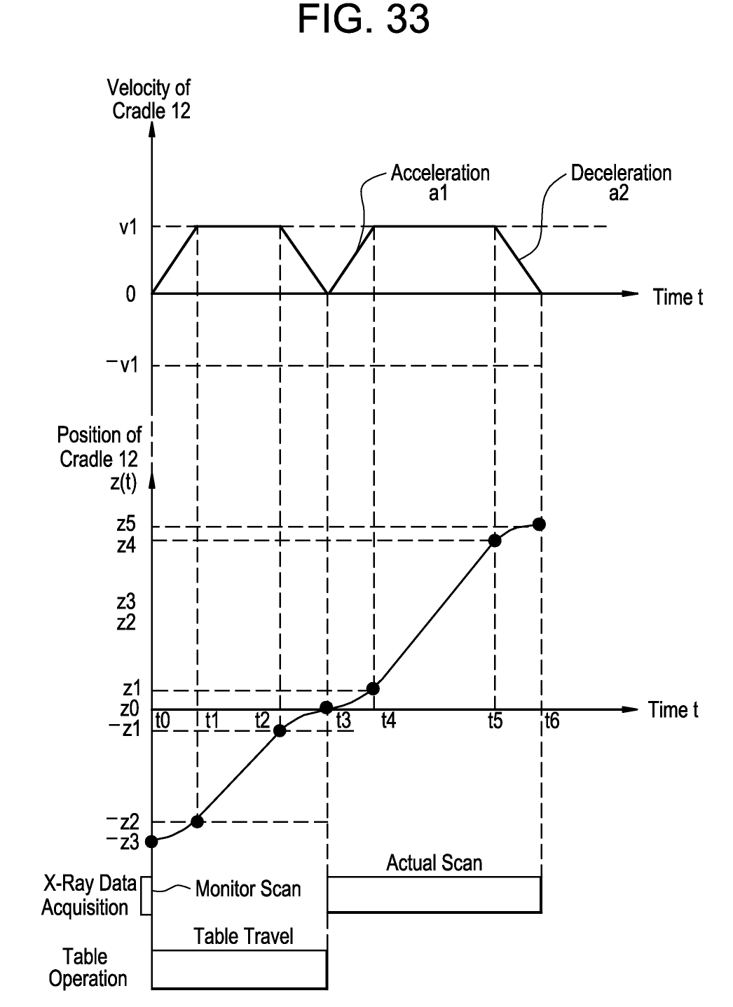

FIG. 32 shows the relative operations between the cradle 12, X-ray tube 21 and multi-row X-ray detector 24 at the actual scan preparatory operation 1.

At times [t0, t1], the cradle is accelerated between velocities 0 and −v1 and moved from z3 to z2 in the z-axis direction.

At times [t1, t2], the cradle is moved at a constant velocity −v1 and shifted from z2 to z1 in the z-axis direction.

At times [t2, t3], the cradle is decelerated between the velocities −v1 and 0 and moved from z1 to z0 in the z-axis direction.

At times [t3, t4], the cradle is accelerated between the velocities 0 and v1 (acceleration a1), and the actual scan is performed while the cradle is being moved from z0 to z1 in the z-axis direction.

At times [t4, t5], the cradle is moved at a constant velocity v1, and the actual scan is performed while the cradle is being moved from z1 to z4 in the z-axis direction.

At times [t5, t6], the cradle is decelerated between the velocities v1 and 0 (deceleration a2), and the actual scan is performed while the cradle is being moved from z4 to z5 in the z-axis direction.

Incidentally, the X-ray projection data acquisition for the actual scan at this time is started from the time t3 and the X rays are outputted, and the variable pitch helical scan z-axis direction movement for the actual scan is made since its start. Although the example of the variable pitch helical scan is shown in FIG. 32, the helical shuttle scan may be used.

<<Actual Scan Preparatory Operation 2>>

X-ray data acquisition of the actual scan preparatory operation 2 is that the cradle is moved in the z-axis direction from a monitor scan position and an actual scan is performed without it turning back. At the actual scan, it is photographed by the variable pitch helical scan as well as <<Actual scan preparatory operation 1>>.

In this case, as well as <<Actual scan preparatory operation 1>>, X-ray projection data acquisition for the actual scan can be brought or reached in a short period of time.

FIG. 33 shows the relative operations between the cradle 12, X-ray tube 21 and multi-row X-ray detector 24 at the actual scan preparatory operation 2.

At times [t0, t1], the cradle is accelerated between velocities 0 and −v1 and moved from −z3 to −z2 in the z-axis direction.

At times [t1, t2], the cradle is moved at a constant velocity v1 and shifted from −z2 to −z1 in the z-axis direction.

At times [t2, t3], the cradle is decelerated between the velocities v1 and 0 and moved from −z1 to −z0 in the z-axis direction.

At times [t3, t4], the cradle is accelerated between the velocities 0 and v1, and the actual scan is performed while the cradle is being moved from z0 to z1 in the z-axis direction.

At times [t4, t5], the cradle is moved at a constant velocity v1, and the actual scan is performed while the cradle is being moved from z1 to z4 in the z-axis direction.

At times [t5, t6], the cradle is decelerated between the velocities v1 and 0, and the actual scan is performed while the cradle is being moved from z4 to z5 in the z-axis direction.

Incidentally, the X-ray projection data acquisition for the actual scan at this time is started from the time t3 and the X rays are outputted, and the variable pitch helical scan z-axis direction movement for the actual scan is made since its start. Although the example of the variable pitch helical scan is shown in FIG. 33, the helical shuttle scan may be used.

<<Actual Scan Preparatory Operation 3>>

X-ray data acquisition of the actual scan preparatory operation 3 is that the cradle is moved in the z-axis direction from a monitor scan position and turned back to perform an actual scan. At the actual scan, it is photographed by the variable pitch helical scan as well as <<Actual scan preparatory operation 1>>.

In this case, as well as <<Actual scan preparatory operation 1>> X-ray projection data acquisition for the actual scan can be reached in a short period of time.

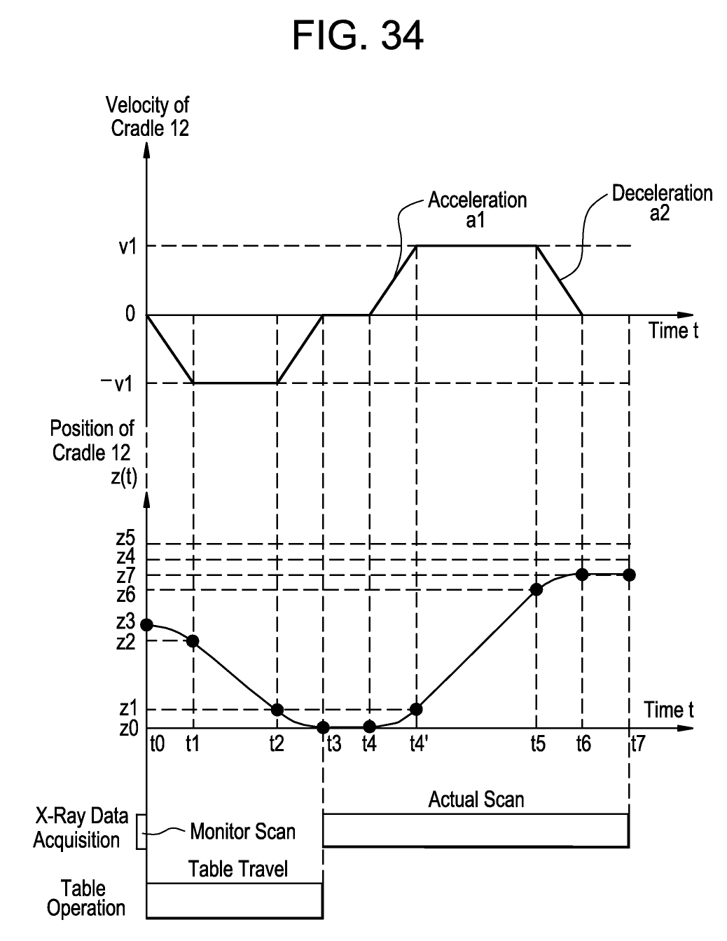

FIG. 34 shows the relative operations between the cradle 12, X-ray tube 21 and multi-row X-ray detector 24 at the actual scan preparatory operation 3.

At times [t0, t1], the cradle is accelerated between velocities 0 and −v1 and moved from z3 to z2 in the z-axis direction.

At times [t1, t2], the cradle is moved at a constant velocity −v1 and shifted from z2 to z1 in the z-axis direction.

At times [t2, t3], the cradle is decelerated between the velocities −v1 and 0 and moved from z1 to z0 in the z-axis direction.

At times [t3, t4], the cradle stays at z=z0, and the actual scan is performed.

At times [t4, t4′], the cradle is accelerated between the velocities 0 and v1 and moved from z0 to z1 in the z-axis direction.

At times [t4′, t5], the cradle is moved at a constant velocity v1, and the actual scan is performed while the cradle is being moved from z1 to z6 in the z-axis direction.

At times [t5, t6], the cradle is decelerated between the velocities v1 and 0, and the actual scan is performed while the cradle is being moved from z6 to z7 in the z-axis direction.

At times [t6, t7], the actual scan is performed while the cradle is staying at z=z7.

Incidentally, the X-ray projection data acquisition for the actual scan at this time is started from the time t3 and the X rays are outputted, and the variable pitch helical scan z-axis direction movement for the actual scan is done since its start. Although the example of the variable pitch helical scan is shown in FIG. 34, the helical shuttle scan may be used.

<<Actual Scan Preparatory Operation 4>>

X-ray data acquisition of the actual scan preparatory operation 4 is that the cradle is moved in the z-axis direction from a monitor scan position and an actual scan is performed without it turning back. Upon the actual scan, however, the actual scan is performed while the cradle is staying in the z-axis direction for a predetermined time interval. At the actual scan, it is photographed by the variable pitch helical scan as well as <<Actual scan preparatory operation 1>>.

In this case as well as <<Actual scan preparatory operation 1>>, X-ray projection data acquisition for the actual scan can be brought in a short period of time.

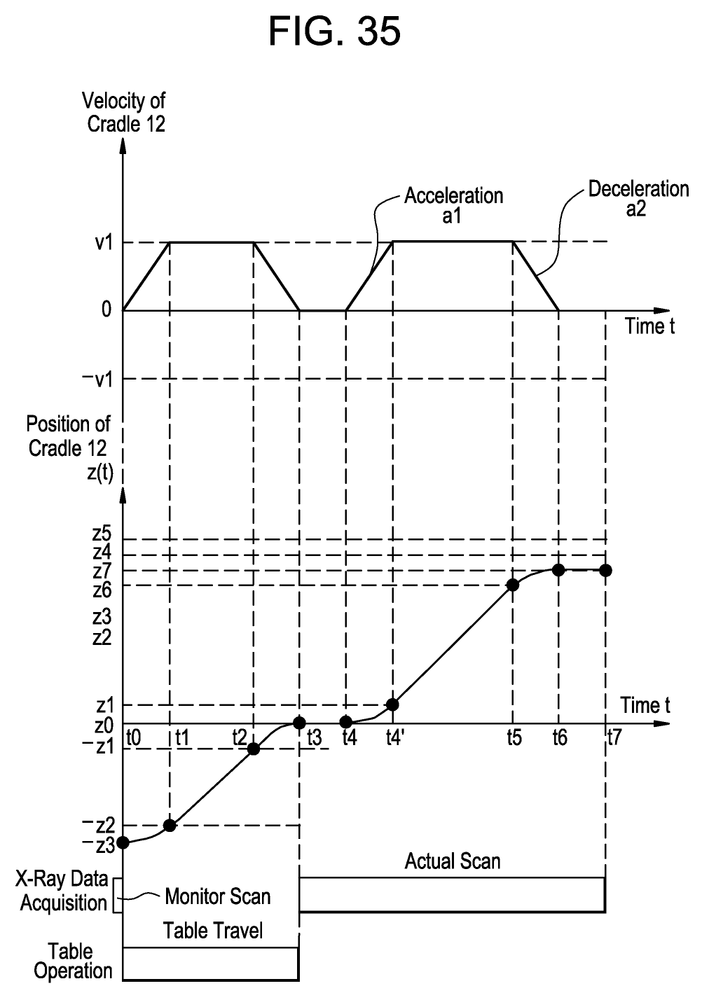

FIG. 35 shows the relative operations between the cradle 12, X-ray tube 21 and multi-row X-ray detector 24 at the actual scan preparatory operation 4.

At times [t0, t1], the cradle is accelerated between velocities 0 and v1 and moved from −z3 to −z2 in the z-axis direction.

At times [t1, t2], the cradle is moved at a constant velocity v1 and shifted from −z2 to −z1 in the z-axis direction.

At times [t2, t3], the cradle is decelerated between the velocities v1 and 0 and moved from −z1 to −z0 in the z-axis direction.

At times [t3, t4], the actual scan is performed while the cradle is staying at z=z0.

At times [t4, t4′], the cradle is accelerated between the velocities 0 and v1 and moved from z0 to z1 in the z-axis direction.

At times [t4′, t5], the cradle is moved at a constant velocity v1, and the actual scan is performed while the cradle is being moved from z1 to z6 in the z-axis direction.

At times [t5, t6], the cradle is decelerated between the velocities v1 and 0, and the actual scan is performed while the cradle is being moved from z6 to z7 in the z-axis direction.

At times [t6, t7], the actual scan is performed while the cradle is staying at z=z7.

Incidentally, the X-ray projection data acquisition for the actual scan at this time is started from the time t3 and the X rays are outputted, and the variable pitch helical scan z-axis direction movement for the actual scan is done since its start. Although the example of the variable pitch helical scan is shown in FIG. 35, the helical shuttle scan may be used.

<<Actual Scan Preparatory Operation 5>>

X-ray data acquisition of the actual scan preparatory operation 5 is that the cradle is moved in the z-axis direction from a monitor scan position and turned back to perform an actual scan. At the actual scan, it is photographed by the variable pitch helical scan as well as <<Actual scan preparatory operation 1>>.

In this case as well as <<Actual scan preparatory operation 1>>, X-ray projection data acquisition for the actual scan can be brought in a short period of time.

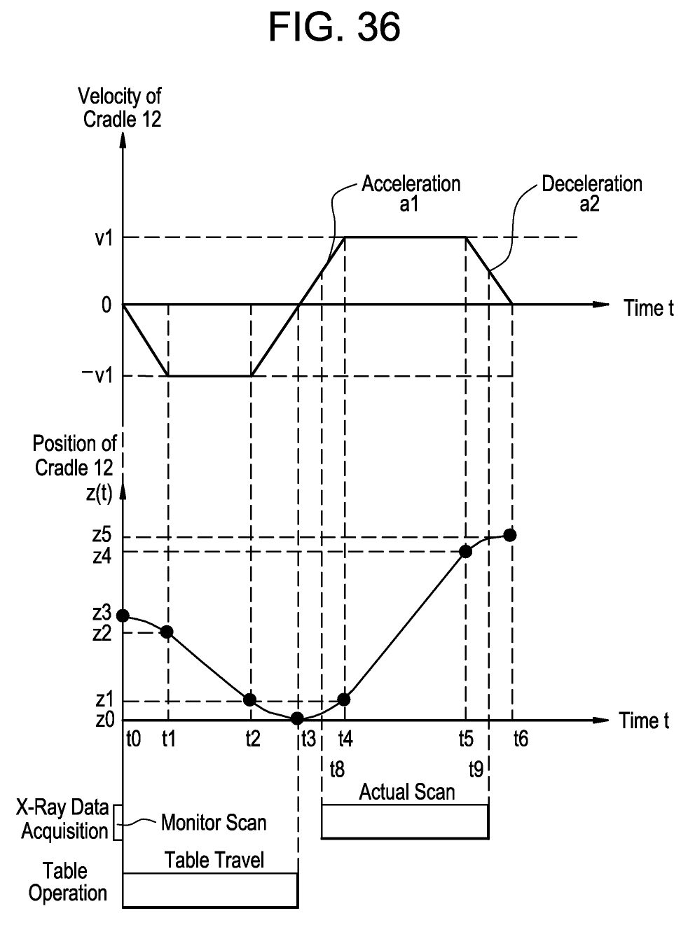

FIG. 36 shows the relative operations between the cradle 12, X-ray tube 21 and multi-row X-ray detector 24 at the actual scan preparatory operation 5.

At times [t0, t1], the cradle is accelerated between velocities 0 and −v1 and moved from z3 to z2 in the z-axis direction.

At times [t1, t2], the cradle is moved at a constant velocity v1 and shifted from z2 to z1 in the z-axis direction.

At times [t2, t3], the cradle is decelerated between the velocities −v1 and 0 and moved from z1 to z0 in the z-axis direction.

At times [t3, t4], the cradle is accelerated between the velocities 0 and v1 and moved from z0 to z1 in the z-axis direction. At this time, X-ray projection data acquisition is started from a time t8.

At times [t4, t5], the cradle is moved at a constant velocity v1, and X-ray projection data acquisition for the actual scan is performed while the cradle is being moved from z1 to z4 in the z-axis direction.

At times [t5, t6], the cradle is decelerated between the velocities v1 and 0 and moved from z4 to z6 in the z-axis direction. At this time, the X-ray projection data acquisition is ended at a time t9.

Incidentally, at this time, X-ray data acquisition by the z-axis direction movement based on relative operations between the cradle 12, X-ray tube 21 and multi-row X-ray detector 24 at the variable pitch helical scan is started at the time t3. Thereafter, X-ray projection data acquisition is started at the time t8, and the X-ray projection data acquisition is completed at the time t9. Afterwards, the z-axis direction movement based on the relative operations between the cradle 12, is stopped at the time t6. Although the example of the variable pitch helical scan is illustrated in FIG. 36, the helical shuttle scan may be used.

X-ray data acquisition at the actual scan preparatory operation 5 is that the X-ray projection data acquisition is started after the z-axis direction movement based on the relative operations between the cradle 12, X-ray tube 21 and multi-row X-ray detector 24 has been performed upon the actual scan. This is equivalent to one in which the timing provided to start the X-ray projection data acquisition for the monitor scan preparatory operation 1 is shifted. The above can be effected even on the monitor scan preparatory operation 2.

<<Actual Scan Preparatory Operation 6>>

X-ray data acquisition of an actual scan preparatory operation 6 is that the cradle is moved in the z-axis direction from a monitor scan position and an actual scan is performed without its turning back. At the X-ray data acquisition of the actual scan, the cradle remains moved at a constant velocity, without carrying out a decelerating operation and an accelerating operation in the neighborhood of an X-ray projection data acquisition start position of a z-axis movement based on relative operations between the cradle 12, X-ray tube 21 and multi-row X-ray detector 24, after which the X-ray projection data acquisition is started.

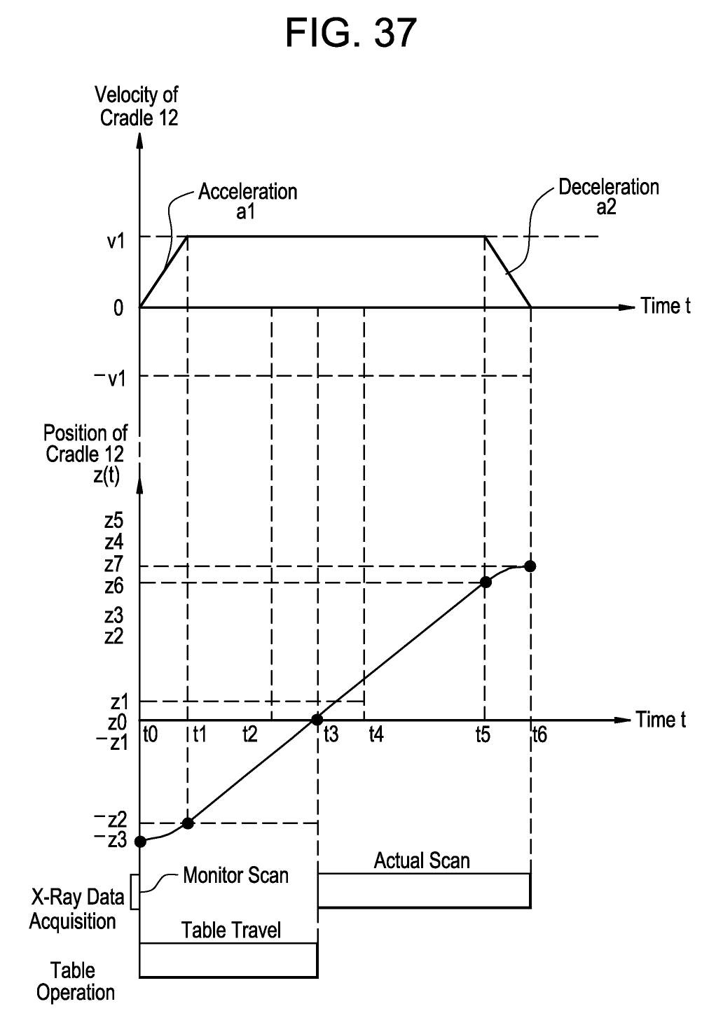

FIG. 37 shows the relative operations between the cradle 12, X-ray tube 21 and multi-row X-ray detector 24 at the actual scan preparatory operation 6.

At times [t0, t1], the cradle is accelerated between velocities 0 and v1 and moved from −z3 to −z2 in the z-axis direction.

At times [t1, t3], the cradle is moved at a constant velocity v1 and shifted from −z2 to −z0 in the z-axis direction.

At the time t3, the cradle keeps moving at a constant velocity without deceleration and X rays are outputted, after which X-ray projection data acquisition is started.

At times [t3, t5], the cradle is moved at the constant velocity v1 and shifted from z0 to z6 in the z-axis direction.

At times [t5, t6], the cradle is accelerated between the velocities v1 and 0 and moved from z6 to z7 in the z-axis direction.

The actual scan can be started by the actual scan preparatory operations from the actual scan preparatory operation 1 to the actual scan preparation operation 6 shown above. Incidentally, such X-ray projection data acquisition and image reconstruction as shown in FIG. 5 are performed upon the actual scan.

The relative operations between the cradle 12, X-ray tube 21 and multi-row X-ray detector 24 from the monitor scan to the actual scan have been described by the embodiment illustrative of the actual scan preparatory operation 1 to 6. However, in any of the actual scan preparatory operation 1 to the actual scan preparatory operation 5, the imaging is performed while the relative operations between the cradle 12, X-ray tube 21 and multi-row X-ray detector 24 are being accelerated thereby making an improvement in timing control on the contrast agent synchronous photography or imaging.

Upon the actual scan preparatory operation 6, an improvement in timing control on the contrast agent synchronous photography is realized by entering the photography for the actual scan without decelerating the relative operations between the cradle 12, X-ray tube 21 and multi-row X-ray detector 24 upon the start of the actual scan photography.

<<Low Exposing Method at Actual Scan>>

Upon the actual scan, a reduction in exposure can be carried out by controlling the X-ray collimator 23 as mentioned below.

Let's first consider where when the scan gantry 20 remains static, the cradle 12 with the subject placed thereon is accelerated and/or decelerated to perform a variable pitch helical scan.

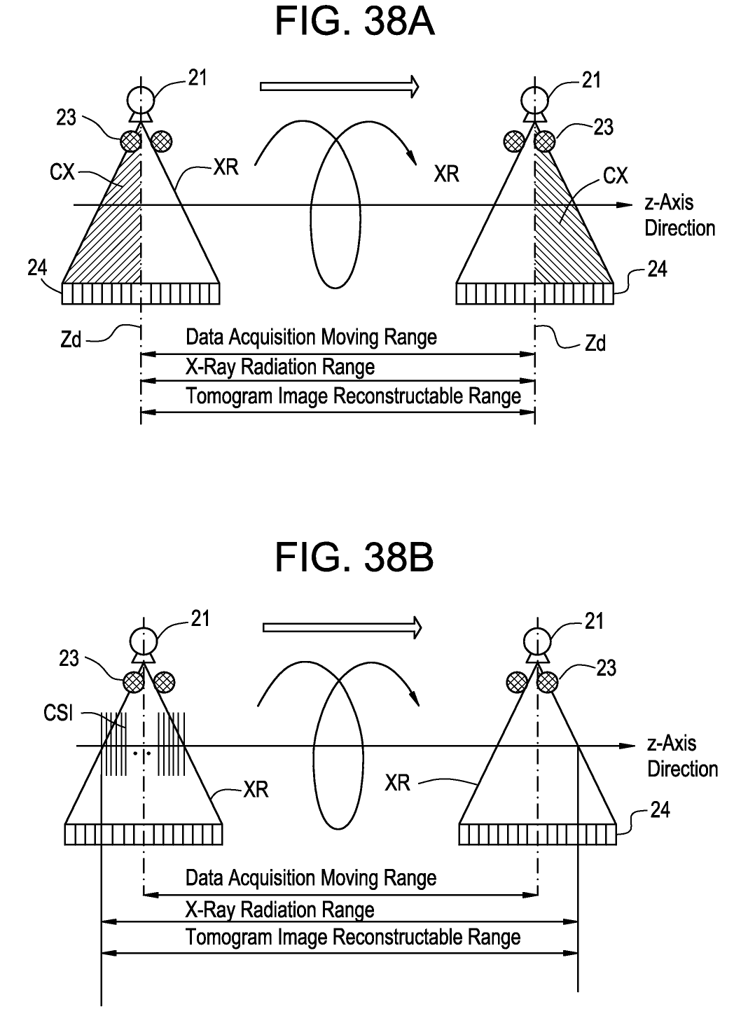

Upon the start of X-ray projection data acquisition of X-ray Collimator 23, as shown in FIG. 38A, an opening or aperture on the side opposite of the cradle 12 is kept closed from the X-ray tube 21 to a z coordinate Zd connecting the center thereof and the center of the multi-row X-ray detector 24. The aperture is gradually opened according to the degree of traveling of the cradle 12 to carry out the X-ray projection data acquisition.

The X-ray collimator 23, as shown in FIG. 38A, is that the aperture on the side of the travel direction of the cradle 12 is gradually closed according to the degree of traveling of the cradle 12 upon the completion of the X-ray projection data acquisition. The X-ray projection data acquisition is performed in such a manner that the aperture of the X-ray collimator 23 is closed to the z coordinate Zd upon the completion of the X-ray projection data acquisition.

Thus, as shown in FIG. 38A, no X rays are applied to a region CX of an X-ray beam XR. That is, a moving range, an X-ray radiation range and a tomogram image reconstructable range of the X-ray tube 21 and multi-row X-ray detector 24 become equal to one another. Therefore, an X-ray needless exposure region becomes nonexistent. It is thus possible to realize a tomogram image reconstruction range that makes the best use of the X-ray radiation range and realize a reduction in exposure.

A specific control method thereof will be shown below.

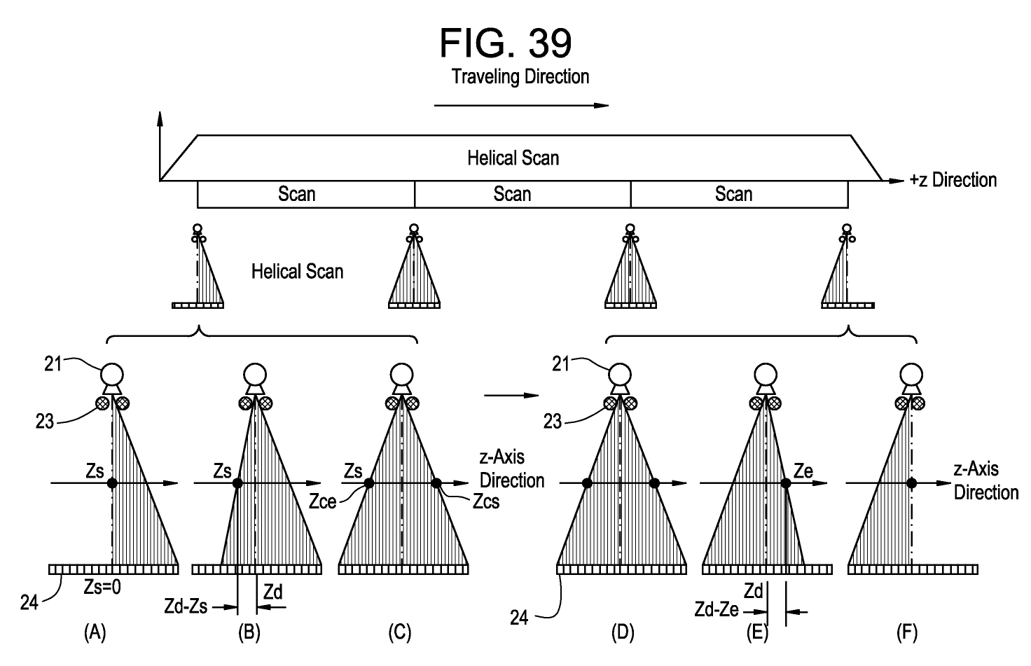

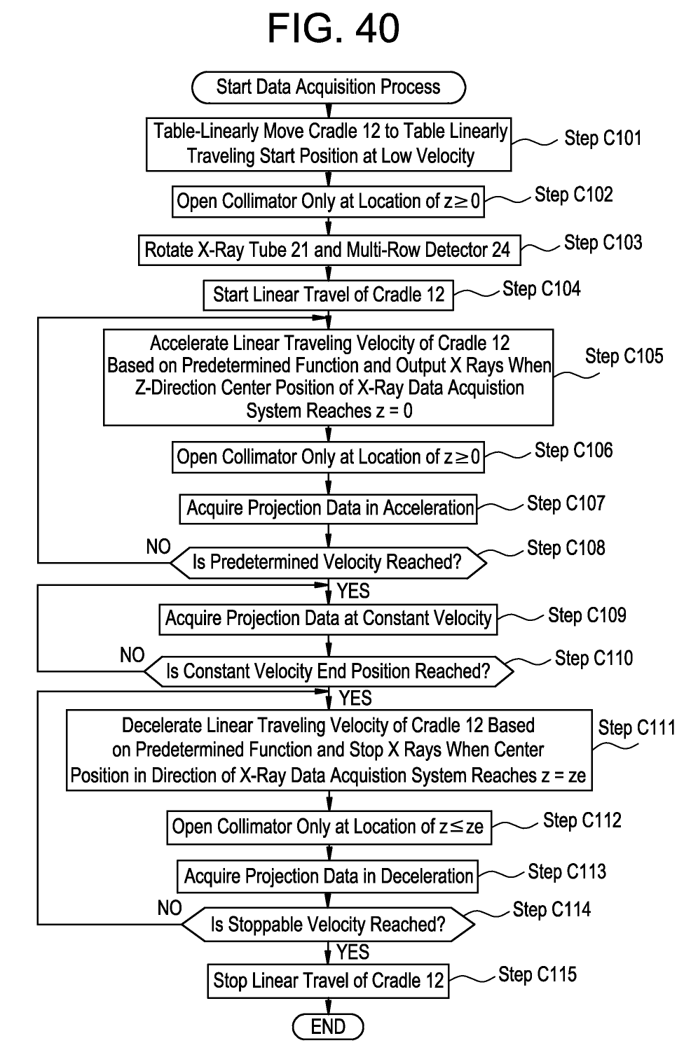

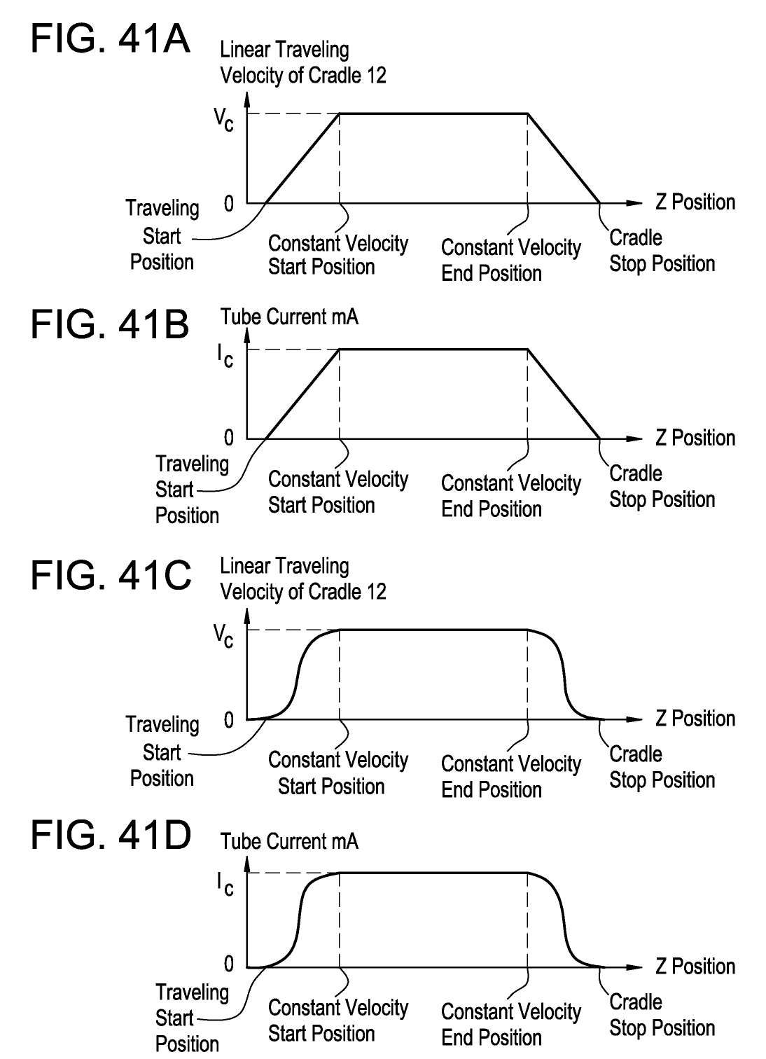

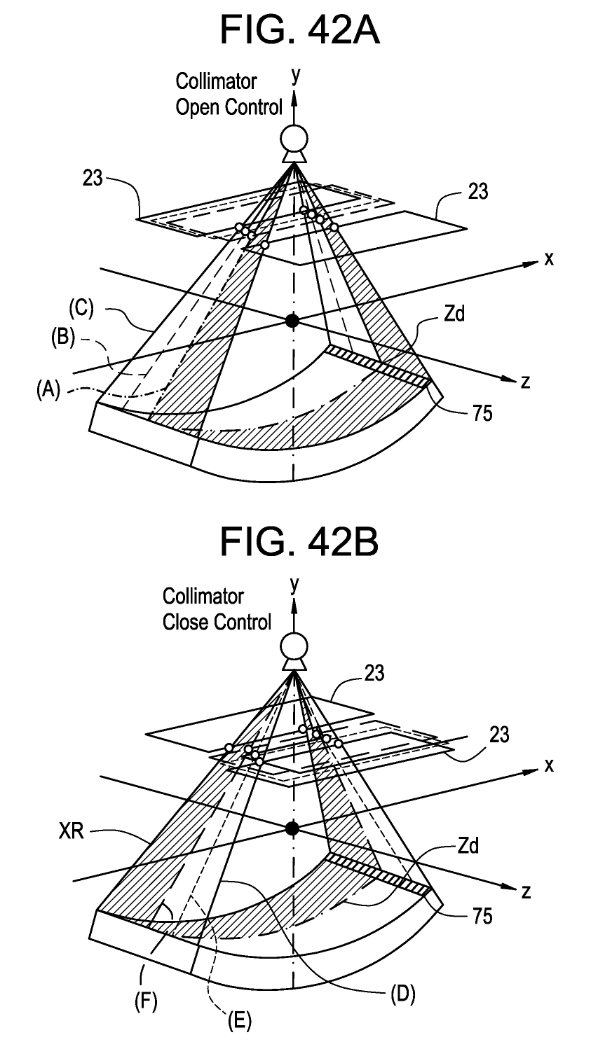

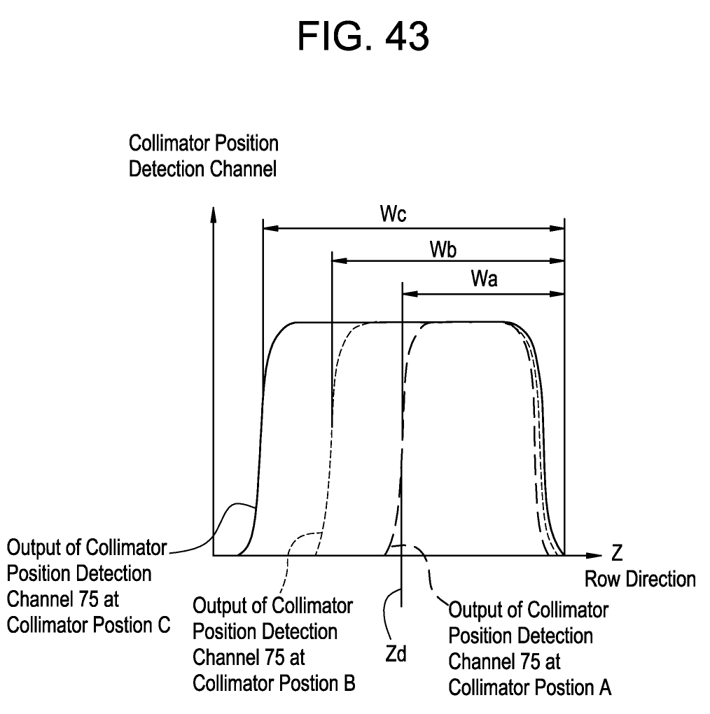

FIG. 39 is an explanatory diagram showing control on the collimator 23 at the X-ray projection data acquisition. Let's assume that in FIG. 39, a center z coordinate of the multi-row X-ray detector 24 is zd, a z coordinate at the start of a helical scan is zs, a z coordinate at the stop of the helical scan is ze, a z coordinate + side of a set slice thickness is zce, a closing/opening width of the collimator 23 is cw, a z coordinate maximum value (+ side) for opening/closing of the collimator 23 is zce, and a z coordinate minimum value (− side) for opening/closing of the collimator 23 is zcs, respectively. FIG. 40 is a flowchart showing the details of processing of control on the collimator 23 at the X-ray projection data acquisition. FIG. 41A is a diagram showing the operation of the cradle 12 subjected to velocity linear control, and FIG. 41C is a diagram showing the operation of the cradle 12 subjected to velocity non-linear control. FIGS. 41B and 41D are respectively diagrams showing an X-ray tube current where velocity linear control is performed, and an X-ray tube current where velocity non-linear control is performed. FIG. 42 is a diagram showing X-ray beams at respective positions of the X-ray collimator 23. FIG. 43 is a diagram showing the output of a collimator position detection channel 75 at each collimator position, i.e., its slice thickness.

At Step C101, the cradle 12 is linearly moved on the table at low velocity up to a table linearly traveling start position shown in FIG. 41A and FIG. 41C.

At Step C102, the collimator 23 is kept open only at a location of z≧0 at the position of the center of rotation IC.

At Step C103, the X-ray tube 21 and the multi-row X-ray detector 24 are rotated about the subject with IC as the center of rotation.

At Step C104, a table linear travel of the cradle 12 is started.

At Step C105, a table linear traveling velocity of the cradle 12 is accelerated based on a predetermined function. FIGS. 41A and 41B respectively show the case in which the predetermined function is linearly controlled with respect to the time, and FIGS. 41C and 41D respectively show the case in which the predetermined function is non-linearly controlled with respect to the time. When the central positions in the z-axis direction, reach z=0, X rays are outputted. In addition to it, Controlling parts of collimator also perform the opening/closing of the collimator 23.

The measurement of the degree of opening/closing of the collimator 23 is made through a position detector channel 75 for the collimator 23, which is indicated at collimator 23 positions A, B, C, D, E and F shown in FIG. 42. FIG. 42A shows opening control of the collimator 23, and FIG. 42B shows closing control thereof. A plus direction corresponding to the z-axis direction indicated by arrow corresponds to the direction of traveling of each of the X-ray tube 21 and the multi-row X-ray detector 24. Incidentally, it is understood that the position detector channel 75 for the collimator 23 exists in both ends or one-sided end of the multi-row X-ray detector 24.

The output of the position detector channel 75 is given as shown in FIG. 43 where the output is seen along the z-axis direction (row direction) in FIG. 42A. When the collimator 23 determines widths wa, wb and wc at each of which the output signal of the position detector channel 75 at this time is outputted, whereby the degree of opening/closing of the collimator 23 is found. The width wa is, for example, when the X-ray collimator 23 on the side opposite to the travel direction of the cradle 12 is moved to the position A, brought to half up to the z coordinate Zd. That is, a z-axis coordinate counted by an encoder that determines a z-axis direction coordinate of a table device 10 is calculated as a z-axis coordinate by the control controller 29, which in turn reaches the DAS 25 via the slip ring 30.

The DAS 25 is capable of recognizing the degree of opening/closing of the present collimator 23 from the output of the position detector channel 75 of the collimator 23 and can issue a command to the collimator 23 in such a manner that it opens/closes to an opening/closing target value of the collimator 23, which has been determined from the z coordinate.

Feedback control as to whether the collimator 23 has been moved as specified is as follows. The difference between an opening/closing value of the collimator 23, which is determined from the output of the position detector channel 75 for the collimator 23, and the opening/closing target value of the collimator 23 is determined to create a feedback signal. Then, a command is issued to the collimator 23 to perform the feedback control.

At Step C106, the collimator 23 is kept open only at the location of z≧0. That is, the collimator 23 is controlled in such a manner that zcs and zs becomes zcs=zs=0 as in the position A in FIG. 39 and the collimator position A in FIG. 42.

At Step C107, X-ray projection data D0 (view, j, i) being in acceleration are acquired. The opening control of the collimator 23 is started in such a manner that zcs and zs become zcs=zs.

At Step C108, when the table linear traveling velocity of the cradle 12 reaches a predetermined velocity Vc shown in each of FIGS. 41A and 41C, the control processing proceeds to Step C109. If it is found not to have reached the predetermined velocity Vc, then the control processing returns to Step C104, where the collimator 23 is further accelerated.

At Step C109, constant-velocity X-ray projection data D0 (view, j, i) are acquired in a state in which the table linear traveling velocity of the cradle 12 is maintained at a predetermined velocity.

At Step C110, when the cradle 12 has reached a constant-velocity velocity end position shown in each of FIGS. 41A and 41C, the control processing proceeds to Step C111. If it is found not to have reached the constant-velocity end position, then the control processing returns to Step C109, where constant-velocity X-ray projection data acquisition is kept continuous.

At Step C111, the table linear traveling velocity of the cradle 12 is decelerated based on a predetermined function and correspondingly the X-ray tube current is reduced. FIGS. 41A and 41B respectively show the case in which the predetermined function is linear control, and FIGS. 41C and 41D respectively show the case in which the predetermined function is non-linear control.

When the coordinate zce on the z-axis direction maximum value side of the collimator 23 begins to fall on the coordinate ze at the stop of the helical scan, the collimator 23 starts to perform close control such that zce=ze is reached. When the central coordinate Zd of each of the X-ray tube 21 and the multi-row X-ray detector 24 reaches Zd=ze, the output of X rays is stopped.

At Step C112, the collimator 23 is kept open only at a location of z≧ze. That is, the collimator 23 is controlled such that zce=ze.

At Step C113, X-ray projection data D0 (view, j, i) being in deceleration are acquired.

At Step C114, when the table linear traveling velocity of the cradle 12 reaches a stoppable velocity shown in each of FIGS. 41A and 41C, the flowchart proceeds to Step C115. If it is found not to have reached the stoppable velocity, then the flowchart returns to Step C111, where the cradle 12 is further decelerated.

At Step C115, the table linear travel of the cradle 12 is stopped.

Thus, By synchronize the cradle 12 and collimator control, the actual embodiment can equal the relative travel or moving range and the X-ray radiation range and hence the X-ray needless exposure region becomes nonexistent. That is, this embodiment is thus possible to realize a tomogram image reconstruction range that makes the best use of the X-ray radiation range and realize a reduction in exposure.

### Second Embodiment

At the actual scan preparatory operation 3 shown in FIG. 34 of the first embodiment and the actual scan preparatory operation 4 shown in FIG. 35, the retention or holding period in which the X-ray tube 21 and the multi-row X-ray detector 24, and the cradle 12 are being relatively stopped in the z-axis direction, exists in the times [t3, t4] and times [t6, t7].

When such three-dimensional image reconstruction as shown in FIG. 7 is used in particular, such retention as described above exists when z=z0 and Z=Z7 at the times [t34, t4] and times [t6, t7] shown in FIG. 34 or 35. If the retention period in which the X-ray tube 21 and multi-row X-ray detector 24, and the cradle 12 are being relatively stopped, is for example, fan angles +180° or more, or fan angles +180° or so, then the tomogram image reconstruction range can be extended outside up to about half the X-ray beam width at maximum as compared with the moving range of each of the X-ray tube 21 and multi-row X-ray detector 24. Simultaneously with it, image reconstruction can be performed without so deteriorating the quality of a tomographic image lying outside the moving range of each of the X-ray tube 21 and the multi-row X-ray detector 24. Therefore, as shown in FIG. 38B, the tomogram image reconstructable range can be expanded to the X-ray radiation range, and the irradiated X rays can be used effectively. Further, no X-ray needless exposure becomes nonexistent, thereby making it possible to realize a reduction in X-ray exposure.

### Third Embodiment

In the first embodiment, needless exposure has been reduced by avoiding the application of X rays to the non image-reconstructed portions by the X-ray collimator 23.

In the second embodiment, the tomogram image reconstructable range is extended to the X-ray radiation range by the three-dimensional image reconstruction using the application of X rays during the retention period in which the X-ray tube 21 and the multi-row X-ray detector 25 and the cradle 12 are being relatively stopped in the z-axis direction, thereby reducing needless exposure.

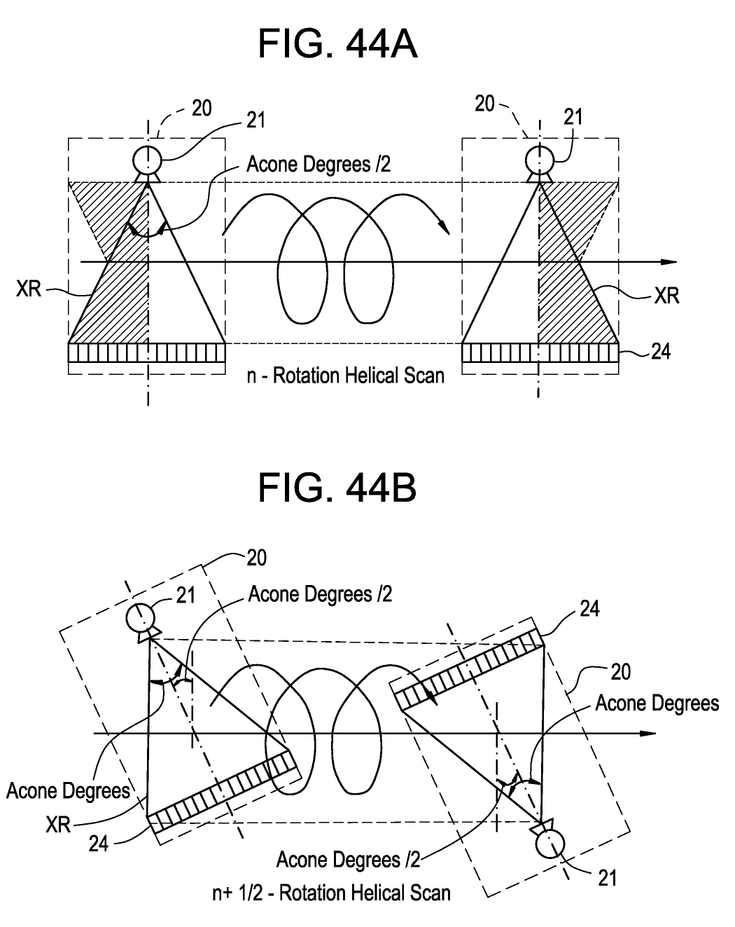

FIG. 44A is a diagram showing an X-ray radiation range for a conventional helical scan, and FIG. 44B is a diagram showing an X-ray radiation range for a helical scan in which a scan gantry 20 of the third embodiment is tilted. At the conventional helical scan, the portions corresponding to diagonally-shaped portions shown at the scan start and end in FIG. 44A have been used as for X-ray radiation unused in image reconstruction. On the other hand, the X-ray radiation unused in such image reconstruction does not exist at the scan start and end in FIG. 44B.

In the third embodiment, the scan gantry 20 is tilted at the start of X-ray data acquisition. The boundary of a rear X-ray beam on the side opposite to the direction of traveling approximately orthogonal to the z-axis direction. Further, the boundary of an X-ray beam lying in the direction of traveling is set approximately orthogonal to the z-axis direction by the end of the X-ray data acquisition. Thus, the shape of the X-ray beam for the X-ray tube 21 and multi-row X-ray detector 24 becomes rectangular within a yz plane as shown in FIG. 44B. In this case of X-ray beam system, the shape of the X-ray beam results in the minimum X-ray radiation shape. The most tomogram image reconstruction therein is performed and a reduction in X-ray exposure can be realized efficiently.

A variable pitch helical scan or a helical shuttle scan with tilted scan gantry 20 is performed in which assuming that the cone angle of the X-ray beam expanded in the z-axis direction is an Acone degrees the scan gantry is tilted by Acone/2°, and X-ray radiation/data acquisition for a helical scan is started, and the X-ray radiation/data acquisition is ended at an n+½ rotation. Incidentally, although the helical scan is performed with the tilt of Acone/2 in the scan gantry 20, an image-reconstructed tomographic image is three-dimensionally image-reconstructed on a plane, i.e., an xy plane orthogonal to the z axis. Thus, both end faces of the X-ray beams at the start and end become orthogonal to the z-axis, and hence needless X-ray exposure can be almost avoided.

Thus, in this way, the control of the X-ray collimator 23, which is synchronized with the operation of the cradle 12 relative to the X-ray data acquisition executed in the first embodiment, is also unnecessary, and hence X-ray needless exposure regions can be eliminated. Using above-mentioned way it makes possible to reduce exposure.

### Fourth Embodiment

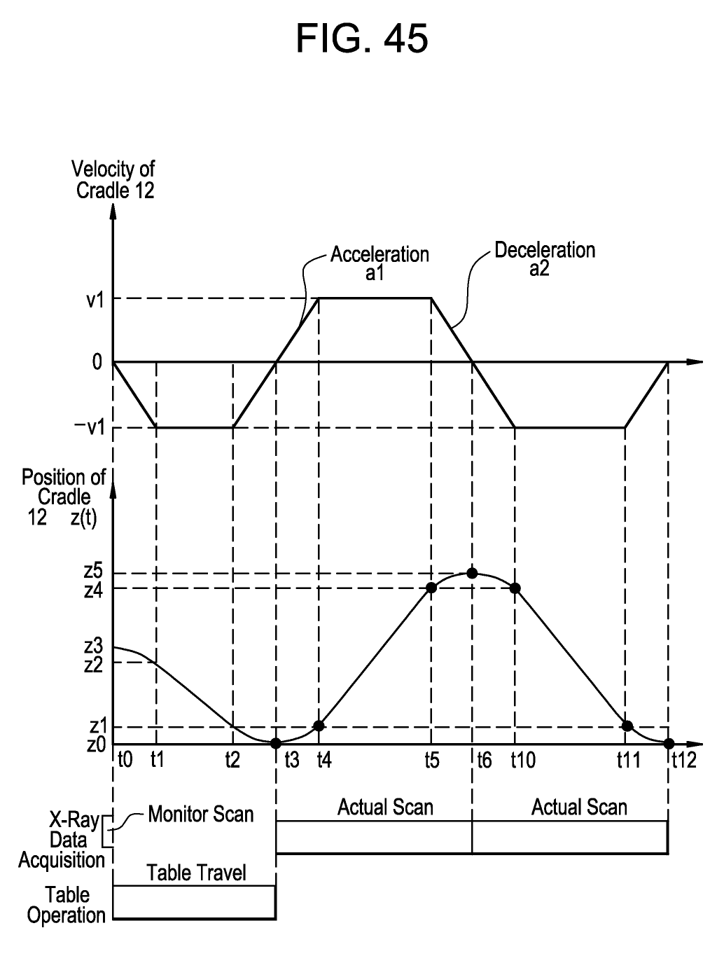

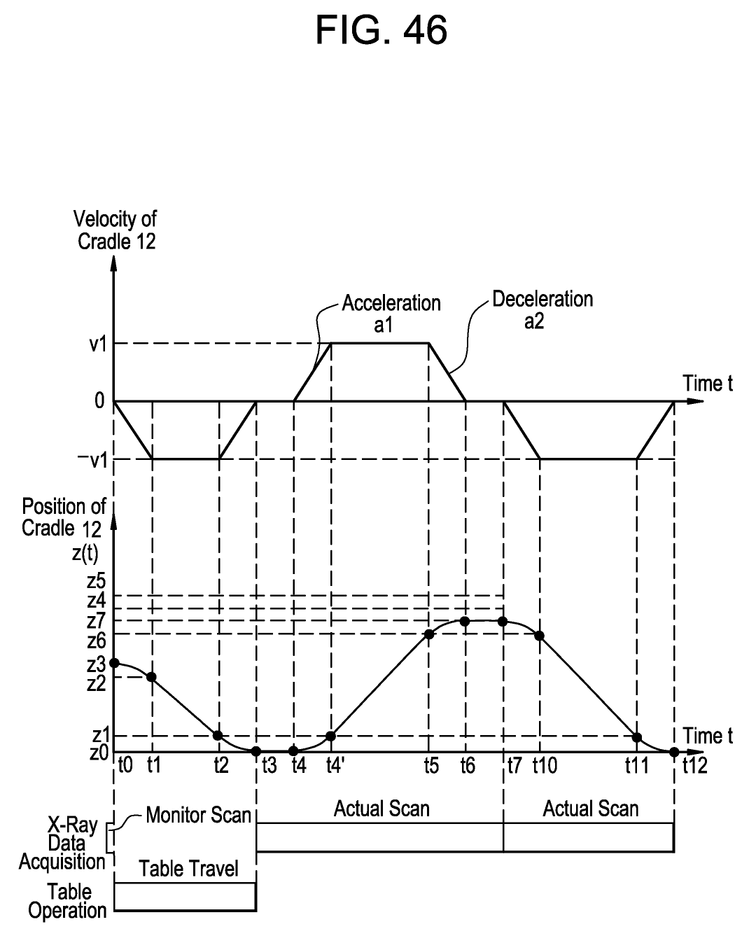

The fourth embodiment shows an example in which an actual scan executes reciprocating motion (±z-axis direction) as well as a-direction (+z-axis direction) in addition to the first embodiment. That is, the present embodiment shows an example in which the actual scan effects the same z-axis direction range plural times as in the helical shuttle scan. Its example is shown in each of FIGS. 45 and 46. FIG. 45 is a diagram showing an actual scan preparatory operation 7 between a cradle 12, and an X-ray tube 21 and a multi-row X-ray detector 24 from a monitor scan MS to an actual scan. FIG. 46 is a diagram showing an actual scan preparatory operation 8 between the cradle 12, and the X-ray tube 21 and the multi-row X-ray detector 24 from a monitor scan to an actual scan.

In FIG. 45, the actual scan is further performed in addition to the case of FIG. 32 of the first embodiment while it is being repeatedly reciprocated at times [t6, t12] from z5 to z0 in a z-axis direction as in the helical shuttle scan.

In FIG. 46, the actual scan is further performed in addition to the case of FIG. 34 of the first embodiment while it is being repeatedly reciprocated at times [t7, t12] from z7 to z0 in the z-axis direction.

In FIG. 45, the X-ray data acquisition may be stopped for a moment at a time t6, or at a time t7 in FIG. 46. Alternatively, the X-ray data acquisition may be performed continuously.

### Fifth Embodiment

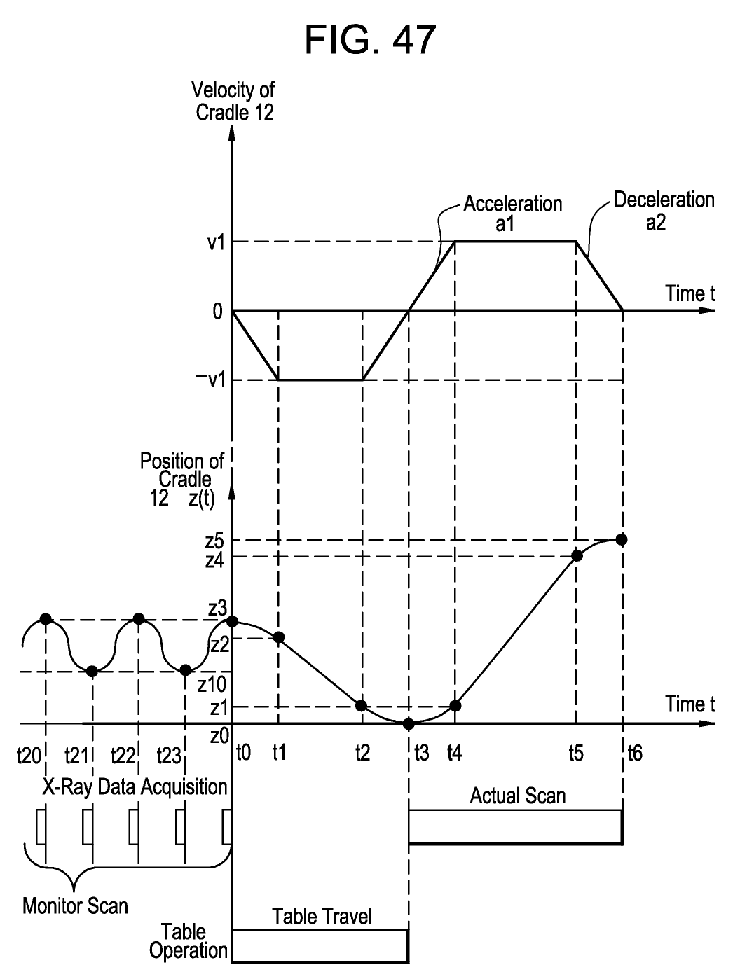

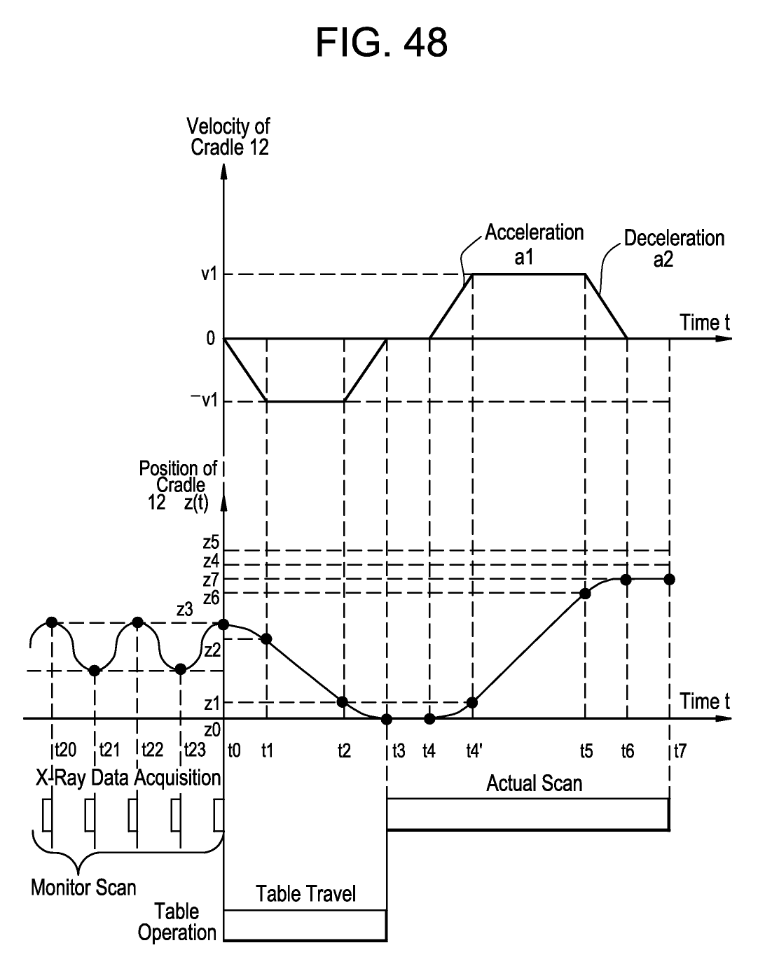

In the fifth embodiment, a monitor scan is performed plural times in advance in addition to the case of the first embodiment. FIG. 47 is a diagram showing an actual scan preparatory operation 9 from a monitor scan MS to an actual scan between a cradle 12 and an X-ray tube 21, and a multi-row X-ray detector 24. FIG. 48 is a diagram showing an actual scan preparatory operation 10 from a monitor scan MS to an actual scan between the cradle 12 and the X-ray tube 21, and the multi-row X-ray detector 24.

When four sheets of tomographic images are photographed in a z-axis direction at a monitor scan where a region of interest ROI set at a monitor scan for contrast agent synchronous photography is set plural in the z-axis direction, for example, each region of interest ROI is set to vascular portions in which a contrast agent for the four sheets of tomographic image flows and a CT value is likely to rise next.

When the plural coordinate positions are separated from one another, there is a need to skip therebetween, preferably, in a short period of time and perform tomographic image photography by a reciprocating operation. If it is not so, then each intermittent scan for such a monitor scan as shown in FIG. 31 cannot be realized.

When the monitor scan positions are away from one another as shown in FIGS. 47 and 48, the tomographic image photography or imaging is performed while reciprocation is repeatedly done as in the helical shuttle scan even during acceleration or deceleration, whereby such intermittent scans as shown in FIG. 31 can be realized.

When some time allowance exists in the interval between the intermittent scans in this case, a standstill or static state corresponding to a predetermined period may be inserted into a period of transition from deceleration to acceleration. Inserting a standstill makes easy on a mechanical control basis and less reduce body movements of the subject. Thus, advantages are the image quality of the monitor scan, particularly, artifacts are reduced. It is thus possible to realize a stable monitor scan good in image quality.

### INDUSTRIAL APPLICABILITY

Incidentally, an image reconstructing method according to the present embodiment may be a three-dimensional image reconstructing method based on the Feldkamp method known to date. Further, it may be another three-dimensional image reconstructing method. Alternatively, it may be two-dimensional image reconstruction. The image quality determined as each portion or region varies in various ways depending upon diagnostic applications, operator's preferences and the like. Therefore, the operator may set in advance imaging condition settings for the optimum image quality at each region or portion.

Although the present embodiment has been described on the basis of the medical X-ray CT apparatus, it can be made available to an X-ray CT-PET apparatus utilized in combination with an industrial X-ray CT apparatus or another apparatus, an X-ray CT-SPECT apparatus utilized in combination therewith, etc.

1. An X-ray CT apparatus comprising:
an X-ray source;
an X-ray detector disposed so as to be opposite to the X-ray source with a subject with a contrast agent injected therein being interposed therebetween;
contrast agent synchronous imaging device which upon start of an actual scan for contrast agent synchronous imaging, acquires projection data while relative operations between the subject and the X-ray source, and the X-ray detector are being accelerated in a predetermined direction; and
image reconstructing device which reconstructs a tomographic image, based on the projection data.

2. The X-ray CT apparatus according to claim 1, wherein the contrast agent synchronous imaging device accelerates the relative operations between the subject and the X-ray source, and the X-ray detector from a static state after the start of acquisition of the projection data.

3. The X-ray CT apparatus according to claim 1, wherein the image reconstructing device image-reconstructs, as a position for image-reconstructing a tomographic image, up to a range extended outside by half an X-ray beam width of an X-ray data acquisition system than a z-direction moving range at a center position of the X-ray data acquisition system.

4. The X-ray CT apparatus according to claim 1, wherein the contrast agent synchronous imaging device acquires the projection data while relative operations between the subject and the X-ray source, and the X-ray detector are being decelerated in a predetermined direction, upon the completion of the actual scan.

5. The X-ray CT apparatus according to claim 1, wherein the contrast agent synchronous imaging device acquires the projection data while relative operations between the subject and the X-ray source, and the X-ray detector are being decelerated in a predetermined direction, upon the completion of the actual scan, acquires the projection data for a predetermined time after the relative operations have been stopped, and thereafter completes the acquisition of the projection data.

6. The X-ray CT apparatus according to claim 4, wherein the contrast agent synchronous imaging device performs deceleration in a predetermined direction at the actual scan and performs acceleration again, and continuously performs the acquisition of the projection data.

7. The X-ray CT apparatus according to claim 5, wherein the contrast agent synchronous imaging device decelerates the relative operations for the actual scan in a predetermined direction and further accelerates the same again after their stop, and continuously performs the acquisition of the projection data.

8. The X-ray CT apparatus according to claim 1, wherein the image reconstructing device performs a three-dimensional image reconstructing process.

9. The X-ray CT apparatus according to claim 1, wherein the image reconstructing device performs image reconstruction using a coordinate position in a predetermined direction, of the projection data, which is obtained by measuring a position in a predetermined direction, of the projection data by coordinate measuring device or predicting a coordinate position in a predetermined direction by relative operations between a subject and an X-ray tube, and an X-ray detector controlled in advance.

10. The X-ray CT apparatus according to claim 1, further including one collimator capable of moving in a traveling direction extending along the predetermined direction from the center connecting an X-ray source and the X-ray detector between the X-ray source and the X-ray detector, and the other collimator capable of moving in a traveling direction opposite to the predetermined direction from the center, wherein the other collimator is moved in the non-traveling direction from the center side upon the start of the actual scan, and the one collimator is moved from the traveling direction to the center upon the end of the actual scan.

11. The X-ray CT apparatus according to claim 1, wherein the contrast agent synchronous imaging device tilts the X-ray source and the X-ray detector with respect to the subject at the start of the actual scan in such a manner that a boundary in a direction opposite to the traveling direction of the predetermined direction, of an X-ray beam emitted from the X-ray source becomes orthogonal to the predetermined direction, and tilts the X-ray source and the X-ray detector with respect to the subject at the end of the actual scan in such a manner that a boundary in the traveling direction, of an X-ray beam emitted from the X-ray source becomes orthogonal to the predetermined direction.

12. The X-ray CT apparatus according to claim 1, wherein the contrast agent synchronous imaging device performs a monitor scan for observing the injection of the contrast agent to the subject before the actual scan, and an X-ray beam width for the monitor scan is narrower than the X-ray beam width for the actual scan.

13. The X-ray CT apparatus according to claim 1, wherein the contrast agent synchronous imaging device performs a monitor scan for observing the injection of the contrast agent to the subject before the actual scan, and performs the actual scan when the maximum value of values of pixels or the mean of plural pixel values thereof selected in decreasing order from the maximum value, said pixels being pixels that belong to a region of interest set by the monitor scan, exceeds a threshold value.

14. The X-ray CT apparatus according to claim 1, wherein the contrast agent synchronous imaging device performs a monitor scan for observing the injection of the contrast agent to the subject before the actual scan, and performs the actual scan when the mean of pixel values in a two-dimensional continuous region, of pixels belonging to a region of interest set by the monitor scan, or the area of the two-dimensional continuous region exceeds a threshold value.

15. The X-ray CT apparatus according to claim 1, wherein the contrast agent synchronous imaging device performs a monitor scan for observing the injection of the contrast agent to the subject before the actual scan, and performs the actual scan when the mean of pixel values in a three-dimensional continuous region, of pixels belonging to a region of interest set by the monitor scan, or the volume of the three-dimensional continuous region exceeds a threshold value.

16. The X-ray CT apparatus according to claim 1, wherein the contrast agent synchronous imaging device performs a monitor scan for observing the injection of the contrast agent to the subject before the actual scan, and the two or more regions of interest set by the monitor scan are included.

17. The X-ray CT apparatus according to claim 1, wherein the contrast agent synchronous imaging device performs a monitor scan for observing the injection of the contrast agent to the subject before the actual scan, and the two or more regions of interest set by the monitor scan are included in the predetermined direction.

18. The X-ray CT apparatus according to claim 12, wherein upon the monitor scan, the acquisition of the projection data is performed while relative operations between the subject and the X-ray source, and the X-ray detector are at least in acceleration or deceleration.

19. The X-ray CT apparatus according to claim 12, wherein upon the monitor scan, the acquisition of the projection data is performed while relative operations between the subject and the X-ray source, and the X-ray detector are in acceleration from a static state or in a static state subsequent to deceleration.

20. An X-ray CT scanning method comprising steps of,
injecting a contrast agent to a subject;
observing a change in CT value at a region of interest after the injection of a contrast agent;
starting an actual scan with the most suitable timing when exceeds an established threshold value of said CT value at a region of interest by acquiring projection data while relative operations between the subject and the X-ray source, and the X-ray detector are being accelerated in a predetermined direction; and
image reconstructing a tomographic image, based on the projection data.
